SEQUENCE LISTING (72 sequences)

SEQ ID NO: 1 - RNA, 21 nt, Artificial Sequence
  siRNA to human fidgetin-like 2, sense strand
uuacacaguauuaaagcgauu

SEQ ID NO: 2 - RNA, 21 nt, Artificial Sequence
  siRNA to human fidgetin-like 2, antisense strand
ucgcuuuaauacuguguaauu

SEQ ID NO: 3 - RNA, 21 nt, Artificial Sequence
  siRNA to human fidgetin-like 2, sense strand
caucugaaaccuagggucuuu

SEQ ID NO: 4 - RNA, 21 nt, Artificial Sequence
  siRNA to human fidgetin-like 2, antisense strand
agacccuagguuucagauguu

SEQ ID NO: 5 - RNA, 21 nt, Artificial Sequence
  siRNA to human fidgetin-like 2, sense strand
gugacuuaugcuaggaggauu

SEQ ID NO: 6 - RNA, 21 nt, Artificial Sequence
  siRNA to human fidgetin-like 2, antisense strand
uccuccuagcauaagucacuu

SEQ ID NO: 7 - RNA, 21 nt, Artificial Sequence
  siRNA to human fidgetin-like 2, sense strand
ggucagaagcagaauguauuu

SEQ ID NO: 8 - RNA, 21 nt, Artificial Sequence
  siRNA to human fidgetin-like 2, antisense strand
auacauucugcuucugaccuu

SEQ ID NO: 9 - RNA, 21 nt, Artificial Sequence
  siRNA to human fidgetin-like 2, sense strand
  misc_feature (20)..(20): n is deoxythymidine
  misc_feature (21)..(21): n is deoxythymidine
cgccggcccacaaguuggann

SEQ ID NO: 10 - RNA, 21 nt, Artificial Sequence
  siRNA to human fidgetin-like 2, antisense strand
  misc_feature (20)..(20): n is deoxythymidine
  misc_feature (21)..(21): n is deoxythymidine
uccaacuugugggccggcgnn

SEQ ID NO: 11 - RNA, 21 nt, Artificial Sequence
  siRNA to mouse fidgetin-like 2, sense strand
  misc_feature (20)..(20): n is deoxythymidine
  misc_feature (21)..(21): n is deoxythymidine
cagcucgagcccuuugacann

SEQ ID NO: 12 - RNA, 21 nt, Artificial Sequence
  siRNA to mouse fidgetin-like 2, antisense strand
  misc_feature (20)..(20): n is deoxythymidine
  misc_feature (21)..(21): n is deoxythymidine
ugucaaagggcucgagcugnn

SEQ ID NO: 13 - RNA, 21 nt, Artificial Sequence
  siRNA to rat fidgetin-like 2, sense strand
  misc_feature (20)..(20): n is deoxythymidine
  misc_feature (21)..(21): n is deoxythymidine
ccuccaaccuccucaagagnn

SEQ ID NO: 14 - RNA, 21 nt, Artificial Sequence
  siRNA to rat fidgetin-like 2, antisense strand
  misc_feature (20)..(20): n is deoxythymidine
  misc_feature (21)..(21): n is deoxythymidine
cucuugaggagguuggaggnn

SEQ ID NO: 15 - RNA, 21 nt, Artificial Sequence
  siRNA to pig fidgetin-like 2, sense strand
  misc_feature (20)..(20): n is deoxythymidine
  misc_feature (21)..(21): n is deoxythymidine
cguugcugcucaucagcgann

SEQ ID NO: 16 - RNA, 21 nt, Artificial Sequence
  siRNA to pig fidgetin-like 2, sense antisense strand
  misc_feature (20)..(20): n is deoxythymidine
  misc_feature (21)..(21): n is deoxythymidine
ucgcugaugagcagcaacgnn

SEQ ID NO: 17 - DNA, 21 bp, Artificial Sequence
  siRNA to human fidgetin-like 2, sense strand
  misc_feature (1)..(1): n is 2′-fluoruridine
  misc_feature (2)..(2): n is 2′-fluorouridine
  misc_feature (3)..(3): n is 2′-O-methyladenosine
  misc_feature (4)..(4): n is 2′fluorocytidine
  misc_feature (5)..(5): n is 2′O-methyladenosine
  misc_feature (6)..(6): n is 2′fluorocytidine
  misc_feature (6)..(6): n is 2′fluorodeoxycytidine
nnnnnnaguauuaaagcgatt

SEQ ID NO: 18 - DNA, 21 bp, Artificial Sequence
  siRNA to human fidgetin-like 2, antisense strand 3′ end has a phosphodiester cap
ttaaugugucauaauuucgcu

SEQ ID NO: 19 - DNA, 4711 bp, Human
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 20 - protein, 653 aa, Human
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 21 - DNA, 4535 bp, Human
gggtttgaaattccaacatggcagaggctgcagtccgtcttcccttcaaaaacttggaatgatttcaaatcataggcaccttcacttaaccctagcttccattcatcagcaaacacatcggatcgatgctacgctaacctatcgggttctctctccgcgcgttcaggttaaatgaatacctgacgaaagggcccacgtttcaaggcagtgacatttgatagctgagaggaaaagtggctttaatgaaaagcaacctttggaattcctgcttgtgagaaatccaattcagctttttgtgctgccagcaagaaatgatcagtagcaccagtgtttatggcttgaagatgcagtggacgccagagcatgcccagtggccagaacagcactttgacatcacctcaaccactcggtctcctgcccacaaagttgaagcctacagaggtcatctgcagcgcacctatcagtacgcctgggcgaatgatgacatatctgctctgactgcatccaacctactaaaaaaatatgcagagaagtattccggcattttggaaggtcctgtggaccgacccgtactcagcaactattcggacacaccatcaggactagtgaacggtcggaaaaatgaaagtgaaccctggcagccttccttgaattcagaagctgtttatcccatgaactgtgttccggatgttatcactgccagcaaagctggagtcagttcagccctccctccagcagatgtctctgcgagtataggaagctctcctggggtagccagcaacctgacagaacctagttattcaagtagtacctgtggaagccacactgtacccagtcttcatgcagggctcccatctcaggaatatgccccaggatacaacggatcatatttgcattctacttatagtagccagccagcacctgcacttccttcacctcatccgtctcctttgcatagctctgggctactacagcccccaccaccacctcctccgccaccagccttggtcccaggctacaatgggacttctaacctctccagttacagctatccgtctgctagctatcctcctcagactgctgtggggtctgggtacagccctgggggggcaccgcctccgccttcagcgtacctgccttcaggaattcctgctcccacccccctaccccccaccactgttcctggctacacctaccagggccatggtttgacacctattgcaccgtcggctctgacaaacagttcagcaagttctctcaaaaggaaagctttctacatggcagggcaaggagatatggactccagttatggaaattacagctatggccaacagagatctacacagagtcctatgtacagaatgcccgacaacagcatttcaaacacaaatcgggggaatggctttgacagaagtgctgaaacatcatccttagcatttaagccaacgaagcagctaatgtcctctgaacagcaaaggaaattcagcagccagtccagtagggctctgacccctccttcctacagtactgctaaaaattcattgggatcaagatccagtgaatcctttgggaagtacacatcgccagtaatgagtgagcatggggacgagcacaggcagctcctctctcacccaatgcaaggccctggactccgtgcagctacctcatccaaccactctgtggacgagcaactgaagaatactgacacgcacctcatcgacctggtaaccaatgagattatcacccaaggacctccagtggactggaatgacattgctggtctcgacctggtgaaggctgtcattaaagaggaggttttatggccagtgttgaggtcagacgcgttcagtggactgacggccttacctcggagcatccttttatttggacctcgggggacaggcaaaacattattgggcagatgcatcgctagtcagctgggggccacatttttcaaaattgccggttctggactagtcgccaagtggttaggagaagcagagaaaattatccatgcctcttttcttgtggccaggtgtcgccagccctcggtgatttttgttagtgacattgacatgcttctctcctctcaagtgaatgaggaacatagtccagtcagtcggatgagaaccgaatttctgatgcaactggacactgtactaacttcggctgaggaccaaatcgtagtaatttgtgccaccagtaaaccagaagaaatagatgaatcccttcggaggtacttcatgaaacgacttttaatcccacttcctgacagcacagcgaggcaccagataatagtacaactgctctcacagcacaattactgtctcaatgacaaggagtttgcactgctcgtccagcgcacagaaggcttttctggactagatgtggctcatttgtgtcaggaagcagtggtgggccccctccatgccatgccagccacagacctttcagccattatgcccagccagttgaggcccgttacatatcaagactttgaaaatgctttctgcaagattcagcctagcatatctcaaaaggagcttgatatgtatgttgaatggaacaaaatgtttggttgcagtcagtgataacttctttagaaaaaaaaaatgtaatgaatgttggcacacacacataaaacctgctacatagggaatagagcccctttccagtagagtttaaattgcaaagggtactggggaagatgacgattaagttgcatctttagagtcagggtagatttggaggaaaagtgcatcaaatgagagcttctgatttgaaagccccagatgacagaaagcatatgtggatgctcagttctgttcaagctagacaacactcaccaaggagcaaggtgcaagtgtgttgatttcagaaggacatgaacctcgtgtgttgattccattctgctgttctcgagatttagttgctgtcaagtgcctggagtggtgctttattttttgtttgcctcacaattacattggtggcatgtgctaatataaagagctttaacttcaaacattattggactaaagagatgaacagttgtgttatgacagaaaaccagatttttgccattttaagagcaacagtattcctcaatcctgtctgttctgcagtattaagctaagaacaggtaaaacagggtaacggtaatctggaccttaatttctgcagttcatttcttttaatgttcttgtctgcaaaaactcaggaaagtgattgtgatttgtacagtacctcaaaggaatgtgttgaaagcactatgtactgctgagagtaataggataggcttcaatgttactttatattaaaatgtatgtttacctcaacaattggaaaatagcaaggaaaattactttgaatgtatccagaaaaatactgaagtgtgatacaactgaatatttacagtttaaagtagaaatggaaggatttttttaagttcttttactaattatggggaattaaccagagcagaataattctttatgtcaataactgcaagagttcttagtacattgctccttgataattaagtgaaaatgttcttaaaaggtacactggttaattgaaagctacttattcagtttgtgttagtgtctagacctgtcagccacaagacctgtttaggaccctgaaagtcacagtacctaaaaactatgactgcctttttattgcataggtggtagtggtggtgatggtggtggtagtttgcaagttatctcttaaaactgctgggaatggtgtcattctattcactaatctagcttatagacttgccgtgctgtttgatagaatgcagaggatagcaaccaaaacaaatacacaaataaataaaaacaaaaaccaaccaacaaaccaacttacatacacatatatatatccacaaagaacctctccatctcctccccttctttttgactccactcttgtcagtgcaattttgcttctcattttgaaatctgggctgtagtgctcctgctttatttctacctcagttttgttacatttctcttggaaagtaaagtagaaaattggaagtggacacacacactgcaatgtagcttgccaaacatgttactttgttttcttccatctttcaccgtaaatctagtttccaaagacatcagcatttgtgcttacttccacctcagtctaccagccccacccctacccatggcataagtggcatttttcttaatttcctatttttctcctgctctctgtcaagttgttctttgtatcctttaatgctttatgtgcaacctttcattgatagtgggctgatgtttggcaatgcttctgaactgtcacagagcaggctgtagctttccacagccactgcccatgcataagcagaacagcctggccttttgaatgtattttcctgggttttttccccttttctttttttagtttagagatgcagtaacaaaactgttgcaaagcactggcattttatgtattcaataaataagtgatgtacatttttaaaaaaatttaaataaatgcaatgagaagccccaagaaag

SEQ ID NO: 22 - protein, 759 aa, Human
MISSTSVYGLKMQWTPEHAQWPEQHFDITSTTRSPAHKVEAYRGHLQRTYQYAWANDDISALTASNLLKKYAEKYSGILEGPVDRPVLSNYSDTPSGLVNGRKNESEPWQPSLNSEAVYPMNCVPDVITASKAGVSSALPPADVSASIGSSPGVASNLTEPSYSSSTCGSHTVPSLHAGLPSQEYAPGYNGSYLHSTYSSQPAPALPSPHPSPLHSSGLLQPPPPPPPPPALVPGYNGTSNLSSYSYPSASYPPQTAVGSGYSPGGAPPPPSAYLPSGIPAPTPLPPTTVPGYTYQGHGLTPIAPSALTNSSASSLKRKAFYMAGQGDMDSSYGNYSYGQQRSTQSPMYRMPDNSISNTNRGNGFDRSAETSSLAFKPTKQLMSSEQQRKFSSQSSRALTPPSYSTAKNSLGSRSSESFGKYTSPVMSEHGDEHRQLLSHPMQGPGLRAATSSNHSVDEQLKNTDTHLIDLVTNEIITQGPPVDWNDIAGLDLVKAVIKEEVLWPVLRSDAFSGLTALPRSILLFGPRGTGKTLLGRCIASQLGATFFKIAGSGLVAKWLGEAEKIIHASFLVARCRQPSVIFVSDIDMLLSSQVNEEHSPVSRMRTEFLMQLDTVLTSAEDQIVVICATSKPEEIDESLRRYFMKRLLIPLPDSTARHQIIVQLLSQHNYCLNDKEFALLVQRTEGFSGLDVAHLCQEAVVGPLHAMPATDLSAIMPSQLRPVTYQDFENAFCKIQPSISQKELDMYVEWNKMFGCSQ

SEQ ID NO: 23 - DNA, 56 bp, Artificial Sequence
  shRNA to human fidgetin-like 2
caccgctggagccctttgacaagttctcgagaacttgtcaaagggctccagctttt

SEQ ID NO: 24 - RNA, 19 nt, Artificial Sequence
  siRNA to human fidgetin
acuagugaacggucggaaa

SEQ ID NO: 25 - RNA, 19 nt, Artificial Sequence
  siRNA to human fidgetin
gcagagaaguauuccggca

SEQ ID NO: 26 - RNA, 19 nt, Artificial Sequence
  siRNA to human fidgetin
ggccaugguuugacaccua

SEQ ID NO: 27 - RNA, 19 nt, Artificial Sequence
  siRNA to human fidgetin
aguugaggcccguuacaua

SEQ ID NO: 28 - DNA, 13024 bp, Human
gtcagtccccgcgcttttcggaggctgccagcgtcccacaccagccgcaggtaggatgcggtgacgggaggcggggcaggggcgctgctgacccctgtggagcatcagggttggggcgtaggggagaggcgggcgtgtgcctctggaatgctgggggtcgcggggcggtgacttcgcgggaggccgtgggaggcgggagccaggcagtggctgtgcccttttgcttctgtcttagcgggagggtggctgacccgctagcccagaaccagactcatccaggattaggaagctggggcccggcgtggacggcaggctcggggccaggtctgcgggcggcttcctttggcagagcctgtccgggggcggggccgcttaccgcaggcatttactgggcccagcggggctctgggcgcccacagcgggggtcagtcagttcccctgtgacccaggcaagctcttaatctctgtgatgagaaagaagggctcatacctttcaggggtcatcgtgaggaatcagaagaagcagtattttatagagtattttctaagtgctgagtcctctgtctagtgctttgcagaattaaaataccttgtcagctcagtgatgttgctgcgcttcttttataggtgaaaaccggcagaaagacattaagagattttcctgcagtcactgctggcagatgatagagccaggatttgaaagcaggcagcctggctccagaccctgtgctcttaactcccgttttgcatcaagaacagaatcctatgaaaggcttgcacagtgcttggtactgagtaggcactccgtcagtattttagtagctattattccaaaggacactgtattctaggtactaagacaatgtgaatggaatggaaaacaattcctccctgcagaagccaactgtctaggaagacagataatgacattattaaaataatcagaacattggcaaaataaagaagattgaaatgcagttcatatgacagtgctaattaatcatactttttagatctaggaaaggccataaaggttaaattgacttggaaatggtaaacaagacaatggttgtgtcactgatcggtgatggccagtttttggttatccttatttgccatttaaatgattgaattgaaaggaagaagtaagacttggaagatgaatattgaaggaaaggtgaagatggaaaagagaagggggtttattctgagaacagtgactagaactctgaggtatggtagaaatttcaggataacagatgataaaggggaatgaagccatggagaggaagagaatgataatattaattgtgaaagtttgtgtcaagtgttttctgagtcacctttctttcagtttctcatttctagtattgtataaatgagtttaacatcaaaatggtatagtgaaaaaaaggatgaactgcttgacagaagaggtcagatctagttttgactctagcttatttagctttacagctttaaataaattacagtgtctttaaagctctggatcttaagttttctaattgttttttaaacaaagatttgtgaacacctacttacaatgtgacaaatgtctttgtaaggcattatgggagataaaagctgcattaaacctggctgttgtacttacaagtaatttggaaggcaaaatttcaaatacttgaacctaaagtagggtttcttggttttcatttaattgaaatactgttaggaaacaaaaatttttcataaatttttcttgtggatatgtaagtaaattctatatatctagcaatatgatacatctatagttctgtttaaaggtaacaatgtgaggaagcttatgcgaataacttgacagaagaaatctaaagtaaaagcattttcctagtaggctagcttgttctcttatttcatttattgtaacagtttcactttttaataattatgtgaaattaggaaacagatattctagggcattctgtttagggtgatgaacttattcatgaatgttattaaaaccatttaaatatgccatagtttaaatttgtgatccatgctgttcaatctgtgacctaggatagcagcatcaaggagcattgtgtacatgcagaagtgcacagtacctggagtgaaactgcttgtgttcgatttctgataccattcataactggctgtgtgatctcaagtaagttctttaagtctctcagcctcggtttgtacatttgtgtgataaggataatgatagttttcagattattttcatagtcattgcttgatttgattcttccaatggttcctctcactttgcagataagaaaactgaagaaccagctgggcgcggtagctcacgcctgtaatcccagcactttgggaggctgaggcgggtggattacctgaggtcaggagttcaagaccagcctgactaacatggcgaaaccccatctctactaaaaatataaaaattatccaggggtggtggcaggtgcctgtaatcccagctacttgggaagctggggcaggagaagcacttgaacccgcgaggtggaggttgcagtgagctgagagcgccactgccctccagcagcctgggtgacagagcgagacactgtctcaaaaaaaaataataataaaataaaaataaaccatcagtgatagtaaaaagctcacttaagctcattcacctagttttatagatcagacttaaacctcttgtttattttgttttgctttatgtatctatagttccattgtttttaaaatttaaaattaattttaaaccttatgaatgctttccctacagataaaattatgtaactagaacaatttaactcatataaattattatttaatcaagtccttaattttgaagacttcatattgatatgtcttgaatacttgaaatctatagttaactagatatttctcttataacctgtccttataaatctacattataaataaaaagtccagtgtcccttccttcagattagcagaagtttcaaaattacatttaaagtattttctttgttttccttcatactgattttcatttttattgtttgttatagaacctctaaaatgcagacctccagctctagatctgtgcacctgagtgaatggcagaagaattacttcgcaattacatctggcatatgtaccggaccgaaggcagatgcataccgtgcacagatattacgcattcagtatgcatgggcaaactctgagatttcccaggtctgtgctaccaaactgttcaaaaaatatgcagagaaatattctgcaattattgattctgacaatgttgaatctgggttgaataattatgcagaaaacattttaactttggcaggatctcaacaaacagatagtgacaagtggcagtctggattgtcaataaataatgttttcaaaatgagtagtgtacagaagatgatgcaagctggcaaaaaattcaaagactctctgttggaacctgctcttgcatcagtggtaatccataaggaggccactgtctttgatcttcctaaatttagtgtttgtggtagttctcaagagagtgactcattacctaactcagctcatgatcgagaccggacccaagacttcccggagagcaatcgtttgaaactccttcagaatgcccagccacctatggtgactaacactgctaggacttgtcctacattctcagcacctgtaggtgagtcagctactgcaaaattccatgtcacaccattgtttggaaatgtcaaaaaggaaaatcacagctctgcaaaagaaaacataggacttaatgtgttcttatctaaccagtcttgttttcctgctgcctgtgaaaatccacagaggaagtctttttatggttctggcaccattgatgcactttccaatccaatactgaataaggcttgtagtaaaacagaagataatggcccaaaggaggatagcagcctgcctacatttaaaactgcaaaagaacaattatgggtagatcagcaaaaaaagtaccaccaacctcagcgtgcatcagggtcttcatatggtggtgtaaaaaagtctctaggagctagtagatcccgagggatacttggaaagtttgttcctcctatacccaagcaagatgggggagagcagaatggaggaatgcaatgtaagccttatggggcaggacctacagaaccagcacatccagttgatgagcgtctgaagaacttggagccaaagatgattgaacttattatgaatgagattatggatcatggacctccagtaaattgggaagatattgcaggagtagaatttgctaaagccaccataaaggaaatagttgtgtggcccatgttgaggccagacatctttactggtttaaggggaccccctaaaggaattttgctctttggtcctcctgggactggtaaaactctaattggcaagtgcattgctagtcagtctggggcaacattctttagcatctctgcttcatccttaacttctaaatgggtaggtgagggggagaaaatggtccgtgcattgtttgctgttgcaaggtgtcagcaaccagctgtgatatttattgacgaaattgattccttgttatctcaacggggagatggtgagcatgaatcttctagaaggataaaaacagaatttttagttcaattagatggagcaacaacatcttctgaagatcgtatcctagtggtgggagcaacaaatcggccacaagaaattgatgaggctgcccggagaagattggtgaaaaggctttatattcccctcccagaagcttcagccaggaaacagatagtaattaatctaatgtccaaagagcagtgttgcctcagtgaagaagaaattgaacagattgtacagcagtctgatgcgttttcaggagcagacatgacacagctttgcagggaggcttctcttggtcctattcgcagtttacaaactgctgacattgctaccataacaccggatcaagttcgacccatagcttacattgattttgaaaatgcttttagaactgtgcgacctagtgtttctccaaaagatttagagctttatgaaaactggaacaaaacttttggttgtggaaagtaagtgggatacttggaatcaaggcatctctgtagtacagtcttctttattttttagcatagaaagttggggatgtgttaattgtatttttaagaatatattctaagttctgtacttcaaataatagcacagattttacatctgattgacatagtgtatgttaatgtaagttttgctttccagtgattacctgatacgtaagcctatttgaacaaagtgagaatgaacttttgtttctaagaagtctttatcttgaagctatataacatgaaaagtgagctcaaattttttttagttgaagattacatataaagttgtgtctgattaatattcatcttttattgaagaaagtgccttctgatggccacataattcttaatgtcagctagtataatggtttacatttggacaaagtattgcttagtgttatttaagtagatttaagatctcaaagctaaagtgccaatttttactttcttcagccaatttgttacctcttttatggtttttaatttttatcaggactaacattttcagaaatagcaaggtgtgatctagtattagattcacagaactgaaaggttaattaagaatgttggttattttcacgggagcgtgtgatatttaacattaatattttatttgacactacagcctgtaatactgctctctttcaaaaacaagttttcagatttttataacagacccattttgttttatgaaacatgttcattacagaaacatgagaaaatagagataagtaaaaagaataaaagtcactattatcctaccactttgcgggacacactattaatatttaagcatagattcttccagatgttttgttttatacagcagtaggattgtgtaatgtattctctcttgtagcctattcttcaaagcgtatcttcactttccatgttaacataaatgtccttttatgattgtctaggattccatggatggatggaatataattgagtcatctggtagagcgcatataggacaggacgctaggttgtttatcttttgtttattgtaaaggaactatgtgggatgcatttggtgatctttttaaaaaatgtataaatttcttgagtaattcctggattaaagaatatgcactttttaaagcttttggcttgcattctaaattgtcctcttgaaaaagtagttcctgtgtaaaactccatctgcactgaatcagagctatggcagcattgggcagtaggcagttttggtatttgttggcattctttttgcatttattcaaaaaactgaaggtagtgaacgtagagcaatgaacaacacaaatatccttggtctcgtggaggttatatttaagttctaatgttttctcttgtgaacagtgcaatttgaaatgacaaattgaaacacttttagttgttgaatttagacagtttgtgtatatagtttaaatttttattcttggtttgccttctgtttttatgttaaaagattgttgcaatttttagtttttccctttgtaatatgtgattgtctttcattagaattcattttttgttttgttttggttattttgaaacagtctcgctgtgttgcctaggctggagtgcagtggtgtgatctcagctcactgcaacctctgcctcctgggttccagtgattcttgtgcctcagtctcccttgtagctgagaccacaggtttgcaccaccacacccagctaatttttgtatttttaatcgagatggggtttcaccatgttgcccaggctgctctcgaactcctggcctcaaacgatccacccacctcagcctcctgaagtgttgggattacaggcatgagccaccacgcctggccttttttattagaatttggacgttaaacttcttgtcttcagttcttagttatatcttttaacatcctaaaacattccacaatattcattactgtctttatccctttctatttctatataaacagcactataaacttggtttctttggtattgatgtcaaggaattctctagtttttctttcttggtgtttaattgcagatcactcccaccttcttgggtctagctatttagatttatcagcatttgccctctagctgttaatgttttatttagctctgaaatttggtcttaatatttattatcagtttttttcagttacagcttctgccagccctgactttaagctcccttgatcaccagccacagggccacttctttctatggagtttagaaattgtgacaaggagagaaagtctagttggtttagctcatcttggattaagcagagcccatgtctctcttgttagctcaagaaatggtctttcctgaattaaggtctactctgttaaataagaatctaaacctgaaaggaatagtcaggatattttaaagctggaaagaaaatttgagctggggagtcaactgttgtttctacgttataccacagagaagctttctttgtctcaacactttgtgattttgagtgtttctgaagcagttggataggaagcttactgaaactttaagattgcttactctcgccacatgcgattgtcttcatcgaatataataatcttggattaaattatctcaagtgtggcactgccctgtctttagctattgtttcagaaacatgaatgctagcctgatatttaatgtcttcaaaaattacattcccttagtagaaatacacattttactaaacatgtcacttggttatggtattaatgcttaacaattttgatatgaatatggtaattctaatttatgatttcaggccttcttaaatctttattatacatctaataataatatatgatccttttgatatgtattcattctgtggtaaatttctaattttctgaaattttaactcattttctcaattctaaaatatctctcaggtgtgcctttcatattgacaagctggtcttaatggaatcatagtctaaagatcatgggctgtggagccaaatagacctgttgaattctggctctgtcatttactggtgactgttggttacttaattattcaatctcaattctaaagtgagaatagtttatacctgtcttgttggagagtgaggattaaatagtaaaatacagatttaaaaggcctggttcatgttttatgattgtgcctcttctttgaatctgccttgattttgctcttgttgctgtgaaggatcttttcagctattttgtgtggttgtcctagctcattctgcttagtgtttgtggtatttgcatcatattattagagatcatatgccatctatctttagttttgtggaaaaattctaattccaaagaaatatctcttagttatgtttctccttttctacactttagaacttttttaaaggtctgtcccagattattatttttctctactcatgcttatatgttatttattggtctccatttttactctcccagtgtttttttaagggacactttacagcccacctttagggatctgattaaaagaaaggattctatcaaggtccatggcaaaaggacaggttggataaagataacatctttttacttccttcttatattcctagttcttgaccttagttggggagctggtggtgagggagcacattttaaacagtttaagcaggtgaggccatcttgaaggtttgccccagcagcctgggtgaatggagggagtcttgggagaatagataagatgcagtcaccacttttaactcttgcccttaaaaaatacttttacaacacggcttataatggaaagagcattaaaatggacattagaaaactgccagtgattctaaccttagagagtttgtacagactccgtaagatttgtttttttagtgataaagggaaattgtactggtcattccttaataacacatccagatttctgttatcacttaccaaaaatttatgaatttgagaattgcttacacagtatcaggtgctgggtactgggaggtacaaaacaaagtatagtcaggtaagggctaactttattactaatttggctcattataaaaacaacgggaaaacatgaagagagggctttattaaggagttaagtcagcataatggaccacttaatatataataaccacttaagagaaaaagaaaaattaggaagtccacataaggtggcgttttataaaggcaacatgctgttgttaagggcacttgggccattggtgttttgtgacagctgggctcaggcatctcaacaagcagcggtgtgcctgctgaccaacagggcctgccacggccagtgaaacacagctattagagctctctcaatccatttctgctcagaaatgtagagatggcaggggaaacgtagctaagtcccaggttcctcggatatggcgagggcaagcactcccatattataacaacctgtgcagtcagtgagtccagagcagaggccttgtctcctcctttgctcaccttcactcactgccctggactgtcaggccccagttctcatccccatgtgtgatgaagccatgcagtcccagagagggagccatcagcgcttggtagtctcatgctttagtgtcagagttgcaccttacctttgtatccaagaaatgcatgcagctcaactttcccttaaaaggcttctaggaaaaaactaccccagcattttttttcccccagaagaatatattagcttaataataaaaggtatagtaacaaactttccatttaaaatagtggcctaagccacaaatttacctttcctgcccgtatgtgcaaaattttcattgaaatgaaaaaatacacactaagaatcaatccagtgtgatgttggaaaagagagaaggttaccattttcaaatatgacaggtgtttctggaggtcacaaggcaaatggtactgggttgttggaatccctgagctaaataatggctgtgaaaatgccagatgagattggttatctcagcagtgcctgggctgtacccctagcttaggcagcaaaataggctgtgggccagtggtctggtatctgaaaggattgccaagacaagccttgcggagctcatgcccaactgttcttgtggaaccactgggaagtggagttccctaaatcagggggagtcatttacagtcgcctggcttttgtgggccctgcagcctgagaagtgagtcaggatggcaggtacttggttgccacgttagacagaagcacaagtatttgatgaagcagtggaacttcttgatgtaactcactcctgtctcggtaactttttacctacttccacatttttttaagggtcgacccccacttcctacagtttctctatatccttgccactgacacatgcaggcttacgccaagacatagaggaggtcttgctcacgccacagttaacacatctctatccatggctgggcaatatgaaagacaattgctaaggaagctttctcctgtcagcatactgatcacttttaaagtataacttaacattacttccccttgtaaaaatcctccaatagattcccatctaaatgaaaataaaatccaaattccttcctagagccaaatatcatttctcttcactctcctttcttactttccttcatcccactggatcttaaggtgcaacaaacatgtttctcatataggaagtgtggtaggcagaattctgaagatgttcctccaagagacccatcccctgcttattcagtcaaatctaggtactgctgggaagggactttgcagaggtaatgcggttacagacctgcagatagggaggttatcttgaattatttgtgtagggccaatctaatttcacaagtccttaaaagcagagaatgttctctggctggttagagaagaggagggctaaagaggcagaagaaagatgtggcagagaggatgtccaagattccgagcatgagaaggattttctctgaagttgcagctctgtgaatcaggagcccatgcacgaaggctggagagaagtctctaggagctgacaggagcctccagacaatagccaacaaaaaccagaggtgtcagtcctagaactgcatagaagggaattctgccagcaacctaaataagccaggaagcctcacccagaggttccagatgagaatccaggccggctgacaccttgatttcagtcttatgggacccagagcagagaaactggtagagactcctatacttctgatgtacagaactgtggggtcataactgtgtgtggttctaagctgctaagtttatggtagtttgttacagcagcaatgtggggtagggaacattacactgttctttgagtgtggaaagctcttccttacagctgctgcttcaagttagtaaaatataatttctccagcaagacctcctttgaccactggggagatagcctccactaacttccaacacgtgcatgccatcactatcatattatcctacattttttattggtaactgttactaactgaaaatacttatgcttactgtctttgtctctaatcctatttcacacccattagaaagttctttacagctagggactttatgtgcacttcacaaccatgcaagattcatgagacttctgaaaaacccaacatcatgagaggcacccagagaagaagcagagtgattcccatcaaagaaaaccattgattcaggagactgagtagagctttagaaagtctcgatcccagaagacatggtagccaccaaattaaaataggcagctattgtattagtatattgttacataacaatatcaccacaaacttagtgactttaaacaacacacattaatgaccttgtagtttctttcaggcttagctgggtcctctgcttcagggacccacagggctgcaatgaaggtgttggtcaggactgtggtctcatctgaggcttgttggggaaggatccacttctcagctcacatggtggttgggcaacattcagtttcttacagatggctggacagagggcctcagttcttgccatctgttggctggaggttgccctcagttccttgccatgtaggcattccagtgtggctgcttgcttcctcaaagccggcaggggagagagcctttccagtagcacagatgttactatcttctgtaatgtaatcacacaggcaacactttgtcatctttgctgtattctgtagggtgagagcaagacacaggtccttcccactcttagggaagggtattaaaaatgacaactaccaggaggtagggatcatggagcctgcttcagaatctatcacagctgtgaaataaaggcccaggaaacaagaattctaatggttaaaataaaaatcttagtggaagaactgaatttttaaaatggaaagggctgaaacaagatagttaaaaggaagaataagctgtggaattattttacaatcttcagcagacagagctagagtgaggagttaagtagtatgacagattccaggtaactaatatccatataatggggaatctatagaaagtaaaaaggagagaggtagatgataattgcattattgagagcagataattcttctgagtgaaatacatagttcatatcaaaagcatctactgagtgtgtggtgttatatatatatgtaggtttttcatccagggttctttggctcataactcccacagcccttacacagtcttgttataatgttggatgtgttaggcctcaggtaggcctctggtgttctgccctccttccactctaatattctcccacctttctgattgtgggtcttaagactcaaaattccaagaggacagggttcagagagcttcccaatagctgaacatgtggaggttccgggagagtggtgtgcccaggtagggcatggaagcttcaagcctcttcccccataccttgccctagcgtttcttcgtctgtattttttccagtatagagcaataaactggtgaactaagtgtt

SEQ ID NO: 29 - protein, 674 aa, Human
MQTSSSRSVHLSEWQKNYFAITSGICTGPKADAYRAQILRIQYAWANSEISQVCATKLFKKYAEKYSAIIDSDNVESGLNNYAENILTLAGSQQTDSDKWQSGLSINNVFKMSSVQKMMQAGKKFKDSLLEPALASVVIHKEATVFDLPKFSVCGSSQESDSLPNSAHDRDRTQDFPESNRLKLLQNAQPPMVTNTARTCPTFSAPVGESATAKFHVTPLFGNVKKENHSSAKENIGLNVFLSNQSCFPAACENPQRKSFYGSGTIDALSNPILNKACSKTEDNGPKEDSSLPTFKTAKEQLWVDQQKKYHQPQRASGSSYGGVKKSLGASRSRGILGKFVPPIPKQDGGEQNGGMQCKPYGAGPTEPAHPVDERLKNLEPKMIELIMNEIMDHGPPVNWEDIAGVEFAKATIKEIVVWPMLRPDIFTGLRGPPKGILLFGPPGTGKTLIGKCIASQSGATFFSISASSLTSKWVGEGEKMVRALFAVARCQQPAVIFIDEIDSLLSQRGDGEHESSRRIKTEFLVQLDGATTSSEDRILVVGATNRPQEIDEAARRRLVKRLYIPLPEASARKQIVINLMSKEQCCLSEEEIEQIVQQSDAFSGADMTQLCREASLGPIRSLQTADIATITPDQVRPIAYIDFENAFRTVRPSVSPKDLELYENWNKTFGCGK

SEQ ID NO: 30 - RNA, 19 nt, Artificial Sequence
  siRNA to human fidgetin-like 1
agugauuaccugauacgua

SEQ ID NO: 31 - RNA, 19 nt, Artificial Sequence
  siRNA to human fidgetin-like 1
gugcacagauauuacgcau

SEQ ID NO: 32 - RNA, 19 nt, Artificial Sequence
  siRNA to human fidgetin-like 1
gaauaaggcuuguaguaaa

SEQ ID NO: 33 - RNA, 19 nt, Artificial Sequence
  siRNA to human fidgetin-like 1
ggaucaaguucgacccaua

SEQ ID NO: 34 - DNA, 21 bp, Artificial Sequence
  siRNA to human fidgetin-like 2, sense strand
uuacacaguauuaaagcgatt

SEQ ID NO: 35 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (2)..(2): n is 2′O′methylcytidine
nngcuuuaauacuguguaatt

SEQ ID NO: 36 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): n is 2′-O′methyuridine
  misc_feature (1)..(2): phosphorotioate linkage
  misc_feature (1)..(1): 5 phosphodiester cap
  misc_feature (2)..(3): phosphorotioate linkage
  misc_feature (2)..(2): n is 2′-O-methylcytidine
nngcuuuaauacuguguaatt

SEQ ID NO: 37 - DNA, 21 bp, Artificial Sequence
  siRNA to human fidgetin-like 2, antisense strand 5′ phosphodiester cap
  misc_feature (1)..(1): n is 2′-fluorodeoxyuridine
  misc_feature (1)..(1): 5′ phosphodiester cap
  misc_feature (2)..(2): n is 2′-fluorodeoxycytidine
nngcuuuaauacuguguaatt

SEQ ID NO: 38 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): 2′-fluorodeoxyuridine
  misc_feature (1)..(1): 5′ phosphodiester cap
  misc_feature (1)..(2): phosphorothioate bond
  misc_feature (2)..(3): phosphorothioate bond
  misc_feature (2)..(2): 2′-fluorodeoxycytidine
nngcuuuaauacuguguaatt

SEQ ID NO: 39 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 1 n is 2′-O-methyluridine
  misc_feature (1)..(1): 5′ phosphodiester cap
  misc_feature (1)..(2): phosphorothioate bond
  misc_feature (2)..(3): phosphorothioate bond
  misc_feature (2)..(2): n is 2′-O-methylcytidine
  misc_feature (12)..(12): n is 2′-O-methylcytidine
  misc_feature (13)..(13): n is 2′-fluorodeoxyuridine
  misc_feature (14)..(14): n is 2′-O-methylguanosine
  misc_feature (15)..(15): n is 2′-fluorodeoxyuridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nngcuuuaauannnnnnnntt

SEQ ID NO: 40 - DNA, 21 bp, Artificial Sequence
  siRNA to human fidgetin-like 2, sense strand 5′ phosphodiester cap Phosphorothioate bond between nucleotides 1 and 2
  misc_feature (1)..(1): 5′ phosphodiester cap
  misc_feature (1)..(2): phosphorotioate bond
ucgcuuuaauacuguguaatt

SEQ ID NO: 41 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-fluorodeoxycytidine
  misc_feature (3)..(3): n is 2′-O-methylguanosine
  misc_feature (4)..(4): n is 2′-fluorodeoxycytidine
  misc_feature (5)..(5): n is 2′-O-methyluridine
  misc_feature (6)..(6): n is 2′-fluorodeoxyuridine
  misc_feature (7)..(7): n is 2′-O-methyluridine
  misc_feature (10)..(10): n is 2′-fluorodeoxyuridine
  misc_feature (11)..(11): n is 2′-O′-methyladenosine
  misc_feature (12)..(12): n is 2′-fluorodeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nnnnnnnaannnngnnnnntt

SEQ ID NO: 42 - RNA, 21 nt, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is 2′-O-methylcytidine
  misc_feature (18)..(18): n is 2′-O-methylguanosine
  misc_feature (19)..(19): n is 2′-O′-methyladenosine
  misc_feature (20)..(20): n is 2′-O-methyluridine
  misc_feature (21)..(21): n is 2′-O-methyluridine
nnnnnnnnnnnnnnnnnnnnn

SEQ ID NO: 43 - RNA, 20 nt, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O′-methyladenosine
  misc_feature (9)..(9): n is 2′-O′-methyladenosine
  misc_feature (10)..(10): n is 2′-O-methyluridine
  misc_feature (11)..(11): n is 2′-O′-methyladenosine
  misc_feature (12)..(12): n is 2′-O-methylcytidine
  misc_feature (13)..(13): n is 2′-O-methyluridine
  misc_feature (14)..(14): n is 2′-O-methylguanosine
  misc_feature (15)..(15): n is 2′-O-methyluridine
  misc_feature (16)..(16): n is 2′-O-methylguanosine
  misc_feature (17)..(17): n is 2′-O-methyluridine
  misc_feature (18)..(18): n is 2′-O′-methyladenosine
  misc_feature (19)..(19): n is 2′-O′-methyladenosine
  misc_feature (20)..(20): n is 2′-O-methyluridine
nnnnnnnnnnnnnnnnnnnn

SEQ ID NO: 44 - DNA, 21 bp, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is deoxyguanosine
  misc_feature (19)..(19): n is deoxyadenosine
nnnnnnnnnnnnnnnnnnntt

SEQ ID NO: 45 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is 2′-O-methylguanosine
  misc_feature (19)..(19): n is 2′-O′-methyladenosine
nnnnnnnnnnnnnnnnnnntt

SEQ ID NO: 46 - RNA, 19 nt, Artificial Sequence
  siRNA to human fidgetin-like 2, sense strand
uuacacaguauuaaagcga

SEQ ID NO: 47 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
ucgcuuuaauacuguguaatt

SEQ ID NO: 48 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 49 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 5′ phosphodiester cap
  misc_feature (1)..(2): phosphorothioate bond
  misc_feature (2)..(3): phosphorothioate bond
ucgcuuuaauacuguguaatt

SEQ ID NO: 50 - RNA, 19 nt, Artificial Sequence
  siRNA to human fidgetin-like 2, sense strand
  misc_feature (1)..(1): n is 2′-O-methyluridine
  misc_feature (2)..(2): n is 2′-O-methyluridine
nnacacaguauuaaagcga

SEQ ID NO: 51 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
  misc_feature (18)..(18): n is 2′-O′-methyladenosine
  misc_feature (19)..(19): n is 2′-O′-methyladenosine
ucgcuuuaauacugugunntt

SEQ ID NO: 52 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 53 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3 and between nucleotides 19 and 20
  misc_feature (1)..(1): 5′ phosphodiester cap
  misc_feature (1)..(2): phosphorothioate bond
  misc_feature (2)..(3): phosphorothioate bond
  misc_feature (19)..(20): phosphorothioate bond
ucgcuuuaauacuguguaatt

SEQ ID NO: 54 - DNA, 21 bp, Artificial Sequence
  siRNA to human fidgetin-like 2, sense strand
  misc_feature (1)..(1): n is locked uridine
  misc_feature (2)..(2): n is locked uridine
  misc_feature (3)..(3): n is locked adenosine
  misc_feature (4)..(4): n is locked cytidine
  misc_feature (5)..(5): n is locked adenosine
nnnnncaguauuaaagcgatt

SEQ ID NO: 55 - DNA, 21 bp, Artificial Sequence
  siRNA to human fidgetin-like 2, antisense strand 5′ phosphodiester cap
  misc_feature (1)..(1): 5′ phosphodiester cap
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 56 - RNA, 19 nt, Artificial Sequence
  siRNA to human fidgetin-like 2, sense strand
  misc_feature (1)..(1): n is 2′-fluorodeoxyuridine
  misc_feature (2)..(2): n is 2′-fluorodeoxyuridine
  misc_feature (3)..(3): n is locked adenosine
  misc_feature (4)..(4): n is 2′-fluorodeoxycytidine
  misc_feature (5)..(5): n is locked adenosine
nnnnncaguauuaaagcga

SEQ ID NO: 57 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): n is 2′-O-methyluridine
  misc_feature (1)..(1): 5′ phosphodiester cap
  misc_feature (1)..(2): phosphorothioate bond
  misc_feature (2)..(2): n is 2′-O-methylcytidine
  misc_feature (3)..(3): n is 2′-O-methylguanosine
nnncuuuaauacuguguaatt

SEQ ID NO: 58 - DNA, 21 bp, Artificial Sequence
  siRNA to human fidgetin-like 2, antisense strand
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 59 - DNA, 21 bp, Artificial Sequence
  iRNA to human fidgetin-like 2, antisense strand Phosphorothioate bond between nucleotides 1 and 2 and between nucleotides 2 and 3
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 60 - DNA, 21 bp, Artificial Sequence
  siRNA to human fidgetin-like 2, antisense strand Phosphorothioate bond between nucleotides 1 and 2 and between nucleotides 2 and 3
  misc_feature (1)..(1): 1 n is 2′-O-methyluridine
  misc_feature (2)..(2): n is 2′-O-methylcytidine
  misc_feature (12)..(12): n is 2′-O-methylcytidine
  misc_feature (13)..(13): n is 2′-fluorodeoxydeoxyuridine
  misc_feature (14)..(14): n is 2′-O-methylguanosine
  misc_feature (15)..(15): n is 2′-fluorodeoxydeoxyuridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nngcuuuaauannnnnnnntt

SEQ ID NO: 61 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 62 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-fluorodeoxydeoxycytidine
  misc_feature (3)..(3): n is 2′-O-methylguanosine
  misc_feature (4)..(4): n is 2′-fluorodeoxydeoxycytidine
  misc_feature (5)..(5): n is 2′-O-methyluridine
  misc_feature (6)..(6): n is 2′-fluorodeoxydeoxyuridine
  misc_feature (7)..(7): n is 2′-O-methyluridine
  misc_feature (10)..(10): n is 2′-fluorodeoxydeoxyuridine
  misc_feature (11)..(11): n is 2′-O-methyladenosine
  misc_feature (12)..(12): n is 2′-fluorodeoxydeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nnnnnnnaannnngnnnnntt

SEQ ID NO: 63 - DNA, 20 bp, Artificial Sequence
  siRNA to human fidgetin-like 2, antisense strand
  misc_feature (1)..(1): n is 2′-O-methyluridine
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O-methyladenosine
  misc_feature (9)..(9): n is 2′-O-methyladenosine
  misc_feature (10)..(10): n is 2′-O-methyluridine
  misc_feature (11)..(11): n is 2′-O-methyladenosine
  misc_feature (12)..(12): n is 2′-O-methylcytidine
  misc_feature (13)..(13): n is 2′-O-methyluridine
  misc_feature (14)..(15): n is a, c, g, or t
  misc_feature (16)..(16): n is 2′-O-methylguanosine
  misc_feature (17)..(17): n is 2′-O-methyluridine
  misc_feature (18)..(18): n is 2′-O-methyladenosine
  misc_feature (19)..(19): n is 2′-O-methyladenosine
  misc_feature (20)..(20): n is 2′-O-methyluridine
nnnnnnnnnnnnnnnnnnnn

SEQ ID NO: 64 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 65 - DNA, 21 bp, Artificial Sequence
  siRNA to human fidgetin-like 2, antisense strand
ucgcuuuaauacuguguaatt

SEQ ID NO: 66 - DNA, 21 bp, Artificial Sequence
  siRNA to human fidgetin-like 2, antisense strand Phosphorothioate bond between nucleotides 1 and 2 and between nucleotides 2 and 3
ucgcuuuaauacuguguaatt

SEQ ID NO: 67 - DNA, 21 bp, Artificial Sequence
  siRNA to human fidgetin-like 2, antisense strand Phosphorothioate bond between nucleotides 1 and 2
  misc_feature (18)..(18): n is 2′-O-methyladenosine
  misc_feature (19)..(19): n is 2′-O-methyladenosine
ucgcuuuaauacugugunntt

SEQ ID NO: 68 - DNA, 21 bp, Artificial Sequence
  siRNA to human fidgetin-like 2, antisense strand
ucgcuuuaauacuguguaatt

SEQ ID NO: 69 - DNA, 21 bp, Artificial Sequence
  siRNA to human fidgetin-like 2, antisense strand Phosphorothioate bond between nucleotides 1 and 2 and between nucleotides 2 and 3 and between nucleotides 19 and 20
ucgcuuuaauacuguguaatt

SEQ ID NO: 70 - DNA, 21 bp, Artificial Sequence
  siRNA to human fidgetin-like 2, antisense strand
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 71 - DNA, 21 bp, Artificial Sequence
  siRNA to human fidgetin-like 2, antisense strand Phosphorothioate bond between nucleotides 1 and 2
  misc_feature (1)..(1): n is 2′-O-methyluridine
  misc_feature (2)..(2): n is 2′-O-methylcytidine
  misc_feature (3)..(3): n is 2′-O-methylguanosine
nnncuuuaauacuguguaatt

SEQ ID NO: 72 - DNA, 21 bp, Artificial Sequence
  siRNA to human fidgetin-like 2, antisense strand
ucgcuuuaauacuguguaatt

(19) United States
(12) Patent Application Publication
(10) Pub. No.: US 2021/0171957 A1
(43) Pub. Date: Jun. 10, 2021

(54) METHODS AND AGENTS FOR ENHANCING T CELL THERAPIES

(71) Applicant: MICROCURES, INC., Santa Cruz, CA (US)

(72) Inventors: Abhinav DEY, Flushing, NY (US); David James Sharp, Scarsdale, NY (US)

(73) Assignee: MICROCURES, INC., Santa Cruz, CA (US)

(21) Appl. No.: 17/079,380

(22) Filed: Oct. 23, 2020

Related U.S. Application Data
(60) Provisional application No. 62/926,238, filed on Oct. 25, 2019.

(51) Int. Cl. C12N 15/113; A61K 9/00; A61K 35/17; A61P 35/00; C12N 5/0783

(52) U.S. Cl. CPC C12N 15/1137; A61K 9/0009; A61K 35/17; A61P 35/00; C12N 2510/00; C12N 2310/14; C12N 2310/122; C12N 2310/531; C12N 5/0636

(57) ABSTRACT

Methods are described for enhancing the tumor homing or penetration activity of T-cells to improve cancer treatment, by exposing T-cells in vitro, ex vivo or in vivo to an agent that inhibits fidgetin-like 2 activity, such as by using an RNA interference agent.

### CROSS-REFERENCE TO RELATED APPLICATIONS

This application claims priority to U.S. Provisional Application No. 62/926,238, filed Oct. 25, 2019, which is hereby incorporated by reference in its entirety.

### INCORPORATION OF SEQUENCE LISTING

The “.txt” Sequence Listing filed with this application by EFS and which is entitled P-589147-US-SQL-09FEB2021_ST25.txt, is 81.7 kilobytes in size and which was created on Feb. 9, 2021, is hereby incorporated by reference. The sequence listing submitted herewith is identical to the sequence listing forming part of the international application.

### BACKGROUND

Adoptive cell therapy with genetically modified T-cells ex vivo holds the promise of improving outcomes for patients with solid tumors and has the potential to reduce treatment complications for all cancer patients. Although T-cells that express chimeric antigen receptors (CARs) specific for CD19 have had remarkable success for B-cell-derived malignancies, which has led to their approval by the U.S. Food and Drug Administration, CAR T-cells have been less effective for solid tumors. Other adoptive T cell therapies including tumor infiltrating lymphocyte therapy, engineered T cell receptor therapy and natural killer (NK) cell therapy similarly have great potential for therapeutic benefit.

Lack of CAR T-cell efficacy in solid tumors is multifactorial. Major roadblocks include: (1) the availability of targeted antigens and their heterogeneous expression; (2) the homing of T cells to tumor sites and cells, and; (3) the immunosuppressive tumor microenvironment. While there is a major focus by Pharma/Biotech/Academic institutions to address the roadblocks 1 and 3 by expansion of the repertoire of targetable antigens or engineering CAR-T cells to resist the immunosuppressive environment, very limited evidence is available on the efforts of enhancing homing or migration of CAR T-cells to tumor sites and within tumors. In addition, amalgamation approaches to cancer immunotherapies have focused on combination of CAR T-cells with checkpoint blockade, oncolytic viruses, chemotherapy, radiation, and/or small molecules. Although recent successes in checkpoint blockade and oncolytic viruses have shown promise, the remaining combinations are beset with side-effects. Enhancement of the efficacy of CAR T-cell other T cell therapies is desirable.

### BRIEF SUMMARY

In one aspect, a method for enhancing the tumoricidal activity of T cells is provided comprising the step of reducing fidgetin-like 2 (FL2) expression or activity in the T cells. In one embodiment, the tumoricidal activity is against a carcinoma, a sarcoma, a lymphoma, a leukemia, a myeloma or a mixed type tumor. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer. In one embodiment, a mixed type tumor is derived from a single germ cell layer that differentiates into more than one cell type. In one embodiment a mixed type tumor is derived from more than one germ cell layer. In one embodiment, the tumoricidal activity is against a solid tumor. In one embodiment, the tumoricidal activity is against a liquid tumor. In one embodiment, the tumoricidal activity is against a bone marrow tumor. In one embodiment, the tumoricidal activity is against a blood cancer. In one embodiment, enhancing the tumoricidal activity comprises enhancing the migration of T cells toward a tumor site or tumor cells, enhancing the penetration or infiltration of T cells into a tumor, enhancing the penetration or infiltration of T cells into a bodily site comprising tumor cells, or any combination of any of the foregoing. In one embodiment, the T cells are endogenous T cells or from adoptive T cell therapy. In one embodiment, the T cells are autologous T cells, CAR-T cells, tumor infiltrating lymphocytes, engineered T cell receptor lymphocytes, macrophages, microglia, or natural killer cells.

In one embodiment, reducing FL2 expression or activity in the T cells is carried out in vivo, ex vivo or in vitro. In one embodiment, the in vivo reducing FL2 expression or activity comprises administering an inhibitor of FL2 to a subject parenterally, administration into a tissue, administration into an organ, administration intratumorally or adjacent to a tumor. In one embodiment the inhibitor of FL2 is administered in or near a lymph node.

In one embodiment, the ex vivo reducing FL2 expression or activity comprises exposing T cells ex vivo to an inhibitor of FL2. In one embodiment, the T cells are subsequently infused into a subject or into a site within the subject. In one embodiment, the in vitro reducing FL2 expression or activity comprises exposing T cells in vitro to an inhibitor of FL2.

In one embodiment the T cells are autologous, allogeneic, lymphoid progenitors or pluripotent stem cells. In one embodiment, the T cells are obtained from the subject. In one embodiment the T cells are obtained from a cell line. In one embodiment the T cells are obtained from a donor. In one embodiment the subject's T cells are processed into CAR-T cells. In one embodiment, the T cells or CAR-T cells are subsequently infused into a subject or a site within the subject.

In one embodiment, the inhibitor of FL2 is a RNA interference agent. In one embodiment, the RNA interference agent is shRNA or siRNA. In one embodiment, the siRNA comprises a sequence selected from

(SEQ ID NO: 1)
UUACACAGUAUUAAAGCGAUU;
(SEQ ID NO: 2)
UCGCUUUAAUACUGUGUAAUU;
(SEQ ID NO: 3
CAUCUGAAACCUAGGGUCUUU;
(SEQ ID NO: 4)
AGACCCUAGGUUUCAGAUGUU;
(SEQ ID NO: 5)
GUGACUUAUGCUAGGAGGAUU;
(SEQ ID NO: 6)
UCCUCCUAGCAUAAGUCACUU;
(SEQ ID NO: 7)
GGUCAGAAGCAGAAUGUAUUU;
(SEQ ID NO: 8)
AUACAUUCUGCUUCUGACCUU;
(SEQ ID NO: 9)
CGCCGGCCCACAAGUUGGAdTdT;
(SEQ ID NO: 10)
UCCAACUUGUGGGCCGGCGdTdT;
(SEQ ID NO: 11)
CAGCUCGAGCCCUUUGACAdTdT;
(SEQ ID NO: 12)
UGUCAAAGGGCUCGAGCUGdTdT;
(SEQ ID NO: 13)
CCUCCAACCUCCUCAAGAGdTdT;
(SEQ ID NO: 14)
CUCUUGAGGAGGUUGGAGGdTdT;
(SEQ ID NO: 15)
CGUUGCUGCUCAUCAGCGAdTdT;
(SEQ ID NO: 16)
UCGCUGAUGAGCAGCAACGdTdT;
(SEQ ID NO: 17)
fUfUmA fCmAfC AGU AUU AAA GCG ATT;
(SEQ ID NO: 18)
(Phos) U CGC UUU AAU ACU G UG UAA TT;
(SEQ ID NO: 34)
5′-UUACACAGUAUUAAAGCGATT-3′;
(SEQ ID NO: 35)
(Phos) 5′ - mUmCGCUUUAAUACUGUGUAATT-3′;
(SEQ ID NO: 36)
(Phos) 5′ - mU(s)mC(s)GCUUUAAUACUGUGUAATT-3′;
(SEQ ID NO: 37)
(Phos) 5′ - fUfCGCUUUAAUACUGUGUAATT -3′;
(SEQ ID NO: 38)
(Phos) 5′ -fU(s)fC(s)GCUUUAAUACUGUGUAATT-3′;
(SEQ ID NO: 39)
(Phos) 5′ - mU(s)mC(s)GCUUUAAUAmCfUmGfUmGfUmAmATT-
3′;
(SEQ ID NO: 40)
(Phos) 5′ - U(s)CGCUUUAAUACUGUGUAATT-3′;
(SEQ ID NO: 41)
(Phos) 5′ - mUfCmGfCmUfUmUAAfUmAfCmUGmUmGfUmAmATT;
(SEQ ID NO: 42)
5′-mUmUmAmCmAmCmAmGmUmAmUmUmAmAmAmGmCmGmAmUmU-3′;
(SEQ ID NO: 43)
(Phos) 5′ - mUmCmGmCmUmUmUmAmAmUmAmCmUmGmUmGmUmAmA
mUmU-3′;
(SEQ ID NO: 44)
5′ mUmUmAmCmAmCmAmGmUmAmUmUmAmAmAmGdCdGdATT-3′;
(SEQ ID NO: 45)
5′ mUmUmAmCmAmCmAmGmUmAmUmUmAmAmAmGdCmGmATT-3′;
(SEQ ID NO: 46)
5′ UUACACAGUAUUAAAGCGA-3′;
(SEQ ID NO: 47)
(Phos) 5′ - U(s)CGCUUUAAUACUGUGUAATT-3′;
(SEQ ID NO: 48)
(Phos) 5′ - UCGCUUUAAUACUGUGUAATT-3′;
(SEQ ID NO: 49)
(Phos) 5′ - U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′ - U(s)CGCUUUAAUACUGUGUmAmATT-3′;
(SEQ ID NO: 52)
(Phos) 5′ - UCGCUUUAAUACUGUGUAATT-3′;
(SEQ ID NO: 53)
(Phos) 5′ - U(s)C(s)GCUUUAAUACUGUGUAA T(s)T-3′;
(SEQ ID NO: 54)
5′ 1U1U1A1C1ACAGUAUUAAAGCGATT-3′;
SEQ ID NO: 55)
(Phos) 5′ - UCGCUUUAAUACUG1U1G1U1A1A TT -3′;
(SEQ ID NO: 56)
5′ fUfU1AfC1ACAGUAUUAAAGCGA-3′;
or
(SEQ ID NO: 57)
(Phos) 5′ - mU(s)mCmGCUUUAAUACUGUGUAATT -3′,

wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate. For example, in SEQ ID NO:17, fC represents 2′-fluorodeoxy cytidine ribonucleic acid, fU represents 2′-fluorodeoxy uracil ribonucleic acid, and mA represents 2′-O-methyl adenosine ribonucleic acid.

In one embodiment, the siRNA consists of any of the foregoing sequences.

In one embodiment, the siRNA has at least one modification selected from a 3′ overhang, a 5′ overhang, a 5′ phosphorylation, a 2′ sugar modification, a nucleic acid base modification, a phosphate backbone modification, and any combination of any of the foregoing. Any of the siRNA sequences may have a phosphodiester cap.

In one embodiment a shRNA to FL2 comprises the sequence CACCGCTGGAGCCCTTTGACAAGTTCTCGAGAACTTGTCAAAGGGCTCCAGCTTTT (SEQ ID NO:23). one embodiment a shRNA to FL2 consists of the sequence CACCGCTGGAGCCCTTTGACAAGTTCTCGAGAACTTGTCAAAGGGCTCCAGCTTTT (SEQ ID NO:23).

In one embodiment, the FL2 is human FL2. In one embodiment, the siRNA is encapsulated in a nanoparticle. In one embodiment, the siRNA is delivered to T cells by nanoparticles, electroporation/nucleofection, Accel siRNA, a viral vector, peptide, protein or aptamer.

In one embodiment, an immune checkpoint inhibitor is administered to the subject with the T cells. In one embodiment, an immune checkpoint inhibitor is exposed to the T cells ex vivo or in vitro before infusion into the subject.

In one aspect, a method is provided for treating cancer in a subject in need thereof comprising administering to a subject a population of T cells wherein the expression or activity of fidgetin-like 2 (FL2) therein has been reduced. In one embodiment the T cells are tumoricidal against the cancer. In one embodiment, the T cells are targeted to the cancer or to an antigen expressed by the cancer. In one embodiment, the tumoricidal activity is against a carcinoma, a sarcoma, a lymphoma, a leukemia, a myeloma or a mixed type tumor. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer. In one embodiment, the tumoricidal activity is against a solid tumor. In one embodiment, the tumoricidal activity is against a liquid tumor. In one embodiment, the tumoricidal activity is against a bone marrow tumor. In one embodiment, the tumoricidal activity is against a blood cancer. In one embodiment, the T cells are autologous T cells, CAR-T cells, tumor infiltrating lymphocytes, engineered T cell receptor lymphocytes, macrophages, microglia, or natural killer cells.

In one embodiment, reducing FL2 expression or activity in the T cells is carried out ex vivo or in vitro. In one embodiment, the ex vivo reducing FL2 expression or activity comprises exposing T cells ex vivo to an inhibitor of FL2. In one embodiment, the in vitro reducing FL2 expression or activity comprises exposing T cells in vitro to an inhibitor of FL2. In one embodiment, the T cells are subsequently infused into a subject or a site within the subject.

In one embodiment the T cells are autologous, allogeneic, lymphoid progenitors or pluripotent stem cells. In one embodiment, the T cells are obtained from the subject. In one embodiment the T cells are obtained from a cell line. In one embodiment the T cells are obtained from a donor. In one embodiment the subject's T cells are processed into CAR-T cells. In one embodiment, the T cells or CAR-T cells are subsequently infused into a subject or a site within the subject.

In one embodiment, the inhibitor of FL2 is a RNA interference agent. In one embodiment, the RNA interference agent is shRNA or siRNA. In one embodiment, the siRNA has a sequence selected from any of SEQ ID Nos:1-18 or 34-57.

In one embodiment, the siRNA has at least one modification selected from a 3′ overhang, a 5′ overhang, a 5′ phosphorylation, a 2′ sugar modification, a nucleic acid base modification, a phosphate backbone modification, and any combination of any of the foregoing.

In one embodiment, the FL2 is human FL2. In one embodiment, the siRNA is encapsulated in a nanoparticle.

In one embodiment, the siRNA is delivered to T cells by nanoparticle, electroporation/nucleofection, Accel siRNA, a viral vector, peptide, protein or aptamer.

In one embodiment, an immune checkpoint inhibitor is administered to the subject with the T cells. In one embodiment, an immune checkpoint inhibitor is exposed to the T cells ex vivo or in vitro before infusion into the subject.

In one aspect, a method is provided for treating cancer in a subject in need thereof comprising administering to or near a tumor or tumor site or lymph node within the subject an inhibitor of the expression or activity of fidgetin-like 2 (FL2). In one embodiment, the tumoricidal activity is against a carcinoma, a sarcoma, a lymphoma, a leukemia, a myeloma or a mixed type tumor. In one embodiment, the tumoricidal activity is against a solid tumor. In one embodiment, the tumoricidal activity is against a liquid tumor. In one embodiment, the tumoricidal activity is against a bone marrow tumor. In one embodiment, the tumoricidal activity is against a blood cancer. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer. In one embodiment, enhancing the tumoricidal activity comprises enhancing the migration of T cells toward a tumor site or tumor cells, enhancing the penetration or infiltration of T cells into a tumor, enhancing the penetration or infiltration of T cells into a bodily site comprising tumor cells, or any combination of any of the foregoing.

In one embodiment, the inhibitor of FL2 is a RNA interference agent. In one embodiment, the RNA interference agent is shRNA or siRNA. In one embodiment, the siRNA has a sequence selected from any of SEQ ID NOs:1-18 or 34-57.

In one embodiment, the siRNA has at least one modification selected from a 3′ overhang, a 5′ overhang, a 5′ phosphorylation, a 2′ sugar modification, a nucleic acid base modification, a phosphate backbone modification, and any combination of any of the foregoing.

In one embodiment, the FL2 is human FL2. In one embodiment, the siRNA is encapsulated in a nanoparticle.

In one embodiment, the siRNA is delivered by implanted wafer, nanoparticle, electroporation/nucleofection, Accel siRNA, a viral vector, peptide, protein or aptamer.

In one embodiment, an immune checkpoint inhibitor is administered to the subject with the inhibitor of FL2.

In one embodiment, a method is provided for treating cancer in a subject in need thereof comprising the steps of (1) determining whether the activity of T cells to migrate to or penetrate into a solid tumor is reduced, and if such activity is reduced, (2) exposing T cells in vitro or ex vivo to an inhibitor that reduces the expression or activity of FL2, and administering the T cells to the subject, or administering to or near a lymph node, tumor or tumor site within the subject, an inhibitor that reduces the expression or activity of FL2, or any combination thereof.

In one embodiment, the T cells are endogenous T cells or the T cells administered for adoptive T cell therapy.

In one embodiment, the determining whether the activity of endogenous T cells or T cells for administration for adoptive T cell therapy to migrate or penetrate into a solid tumor is carried out by an in vitro T-cell migration assay.

In one embodiment, the cancer is a solid tumor, a liquid tumor, a bone marrow tumor or a blood cancer. In one embodiment, the cancer is a carcinoma, a sarcoma, a lymphoma, a leukemia, a myeloma or a mixed type tumor. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer.

In one embodiment, exposing enhances the migration of T cells toward a tumor site or tumor cells, enhances the penetration or infiltration of T cells into a tumor, enhances the penetration or infiltration of T cells into a bodily site comprising tumor cells, or any combination thereof.

In one embodiment, the T cells are administered for adoptive T cell therapy. In one embodiment, the T cells are autologous T cells, CAR-T cells, tumor infiltrating lymphocytes, engineered T cell receptor lymphocytes, macrophages, microglia or natural killer cells, or are derived from lymphoid progenitor cells or pluripotent stem cells.

In one embodiment, reducing FL2 expression or activity in the T cells is carried out in vivo, ex vivo or in vitro. In one embodiment, the in vivo reducing FL2 expression or activity comprises administering an inhibitor of FL2 to a subject parenterally, into a tissue, into an organ, lymph node, intratumorally or adjacent to a tumor. In one embodiment, the ex vivo reducing FL2 expression or activity comprises exposing T cells ex vivo to an inhibitor of FL2. In one embodiment, the T cells are subsequently infused into a subject or a site within the subject. In one embodiment, the in vitro reducing FL2 expression or activity comprises exposing T cells in vitro to an inhibitor of FL2. In one embodiment, the T cells are subsequently infused into a subject or a site within the subject.

In one embodiment, the T cells are autologous, allogeneic, lymphoid progenitors or pluripotent stem cells. In one embodiment, the T cells are obtained from a cell line or from a donor.

In one embodiment, the inhibitor of FL2 is a RNA interference agent. In one embodiment, the RNA interference agent is shRNA or siRNA. In one embodiment, the siRNA has a sequence selected from any one of SEQ ID NOs:1-18 or 34-57. In one embodiment, the siRNA has at least one modification selected from a 3′ overhang, a 5′ overhang, a 5′ phosphorylation, a 2′ sugar modification, a nucleic acid base modification, a phosphate backbone modification, and any combination of any of the foregoing.

In one embodiment, the FL2 is human FL2.

In one embodiment, the siRNA is encapsulated in a nanoparticle. In one embodiment, the siRNA is delivered by nanoparticles, electroporation/nucleofection, Accel siRNA, a viral vector, peptide, protein or aptamer.

In one embodiment, an immune checkpoint inhibitor is administered to the subject or exposed to T cells in vitro or ex vivo before infusion into the subject.

In one aspect, a method for enhancing the tumoricidal activity of T cells is provided comprising the step of reducing fidgetin-like 1 (FL1) expression or activity in the T cells. In one embodiment, the tumoricidal activity is against a carcinoma, a sarcoma, a lymphoma, a leukemia, a myeloma or a mixed type tumor. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer. In one embodiment, a mixed type tumor is derived from a single germ cell layer that differentiates into more than one cell type. In one embodiment a mixed type tumor is derived from more than one germ cell layer. In one embodiment, the tumoricidal activity is against a solid tumor. In one embodiment, the tumoricidal activity is against a liquid tumor. In one embodiment, the tumoricidal activity is against a bone marrow tumor. In one embodiment, the tumoricidal activity is against a blood cancer. In one embodiment, enhancing the tumoricidal activity comprises enhancing the migration of T cells toward a tumor site or tumor cells, enhancing the penetration or infiltration of T cells into a tumor, enhancing the penetration or infiltration of T cells into a bodily site comprising tumor cells, or any combination of any of the foregoing. In one embodiment, the T cells are endogenous T cells or from adoptive T cell therapy. In one embodiment, the T cells are autologous T cells, CAR-T cells, tumor infiltrating lymphocytes, engineered T cell receptor lymphocytes, macrophages, microglia, or natural killer cells.

In one embodiment, reducing FL1 expression or activity in the T cells is carried out in vivo, ex vivo or in vitro. In one embodiment, the in vivo reducing FL1 expression or activity comprises administering an inhibitor of FL1 to a subject parenterally, administration into a tissue, administration into an organ, administration intratumorally or adjacent to a tumor. In one embodiment the inhibitor of FL1 is administered in or near a lymph node. Other embodiments are as described above.

In one aspect, a method for enhancing the tumoricidal activity of T cells is provided comprising the step of reducing fidgetin expression or activity in the T cells. In one embodiment, the tumoricidal activity is against a carcinoma, a sarcoma, a lymphoma, a leukemia, a myeloma or a mixed type tumor. In one embodiment, the tumor or cancer is colorectal cancer, pancreatic cancer, liver cancer, intrahepatic bile ductal cancer, esophageal cancer, bladder cancer, non-Hodgkin's lymphoma or kidney cancer. In one embodiment the cancer is a rare cancer. In one embodiment, a mixed type tumor is derived from a single germ cell layer that differentiates into more than one cell type. In one embodiment a mixed type tumor is derived from more than one germ cell layer. In one embodiment, the tumoricidal activity is against a solid tumor. In one embodiment, the tumoricidal activity is against a liquid tumor. In one embodiment, the tumoricidal activity is against a bone marrow tumor. In one embodiment, the tumoricidal activity is against a blood cancer. In one embodiment, enhancing the tumoricidal activity comprises enhancing the migration of T cells toward a tumor site or tumor cells, enhancing the penetration or infiltration of T cells into a tumor, enhancing the penetration or infiltration of T cells into a bodily site comprising tumor cells, or any combination of any of the foregoing. In one embodiment, the T cells are endogenous T cells or from adoptive T cell therapy. In one embodiment, the T cells are autologous T cells, CAR-T cells, tumor infiltrating lymphocytes, engineered T cell receptor lymphocytes, macrophages, microglia, or natural killer cells.

In one embodiment, reducing fidgetin expression or activity in the T cells is carried out in vivo, ex vivo or in vitro. In one embodiment, the in vivo reducing fidgetin expression or activity comprises administering an inhibitor of fidgetin to a subject parenterally, administration into a tissue, administration into an organ, administration intratumorally or adjacent to a tumor. In one embodiment the inhibitor of fidgetin is administered in or near a lymph node. Other embodiments are as described above.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts time-lapse images for migration of T-cells (red) towards a cancer cell (green) in a 3D co-culture of Jurkat cells (T-cell line) with DU145 cells (prostate cancer cell line). The T-cells (red) show a directional movement (arrows) towards small clusters of cancer cells (green) (hour 0 to hour 5) and with time show phagocytosis of the cancer cells (hour 6 to hour 13). Both the cell lines were pre-stained with membrane specific lipophilic Vybrant dyes and then co-cultured in a 3D Matrigel plug.

FIG. 2A, FIG. 2B, FIG. 2C, FIG. 2D, FIG. 2E, FIG. 2F, FIG. 2G and FIG. 2H show limited penetration of T-cells in prostate cancer spheroids. FIG. 2A. The Jurkat cells (T-cell line) were co-cultured in a Matrigel plug along with a prostate cancer spheroid derived from DU145 cell line. FIG. 2B. Low magnification image of pre-stained Spheroid (green) and the surrounding pre-stained T-cells (red). FIG. 2C to FIG. 2H. Time lapse series tracking the motility and penetration of T-cells (red). Note the migration of T-cells directed towards the edge of spheroid (arrows) (FIG. 2C-FIG. 2F) and then migration away from the spheroid possibly owing to immunosuppressive hypoxic tumor microenvironment.

FIG. 3A, FIG. 3B and FIG. 3C show FL2 knockdown in T-cells enhances migration to cancer spheroids. FIG. 3A. Western blot showing the knockdown of FL2 in Jurkat cells (T-cell line). FIG. 3B. Schematic of trans-well assay for quantifying T-cell migration towards cancer spheroids. The T-cells modified with siRNA+GFP are placed in the upper chamber and the spheroids are in the lower chamber. The trans-well semipermeable membrane is coated with Matrigel to trap the T-cells migrating towards the spheroids. FIG. 3C. Imaging the GFP-positive cells on the transwell membrane 72 hours after co-culture with cancer spheroids (Scale bar=400 um). D. Quantification of GFP-positive T-cells from images show significantly (*; p=0.0159) higher number of T-cells treated with FL2 siRNA migrating towards the cancer spheroids.

FIG. 4A and FIG. 4B show FL2 knockdown in T-cells enhances migration to different cancer spheroids. FIG. 4A. Imaging the GFP-positive cells on the transwell membrane 72 hours after co-culture with cancer spheroids from Prostate cancer cell lines (DU145 and LnCAP) and Lung Cancer cell line (DMS53) (Scale bar=400 um). FIG. 4B. Quantification of GFP-positive T-cells from images show significantly (***; p<0.001; **; p<0.01) higher number of T-cells treated with FL2 siRNA migrating towards the cancer spheroids.

FIG. 5 depicts evaluation of therapy using FL2 siRNA-treated T cells in a xenograft model.

FIG. 6 depicts a human study in which CAR-Ts are treated with any one of several types of FL2 modifications to inhibit FL2 before infusion.

FIG. 7 depicts a method for determining or quantifying whether engineered T cells have sufficient homing or penetration activity to a patient's tumor, prior to CAR-T cell therapy.

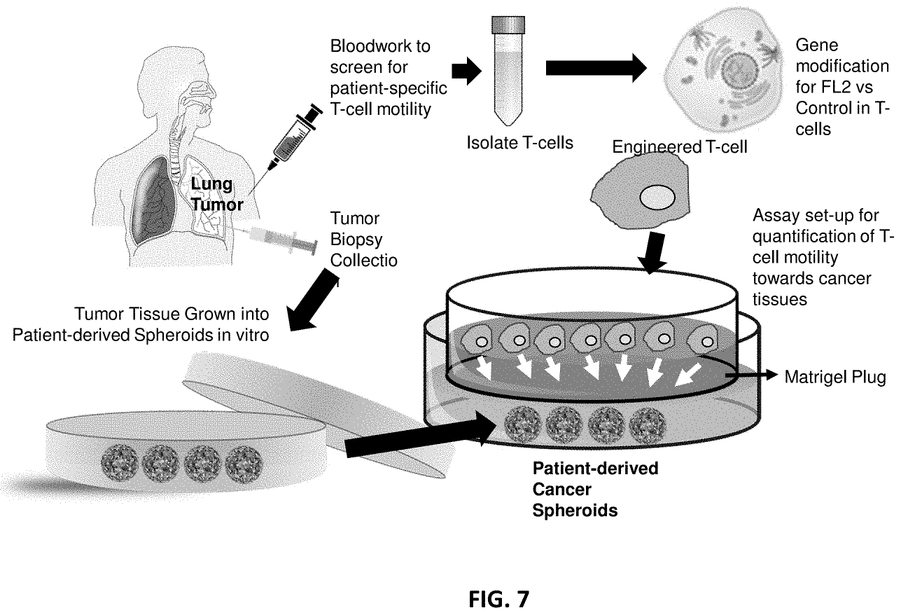

FIG. 8 shows the effect of FL2 knockdown on T cell attachment to a Du145 monolayer.

FIG. 9A-B show the effect of FL2 knockdown on attachment of T cells to tumor spheroids.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

T cell therapy represents a powerful method to harness the body's own immune cells to attack cancer cells. Despite recent advances in immunotherapy, certain tumors and especially solid tumors provide a challenge for attack by T cell therapies. A current need exists to enhance the migration of T cells to and penetration of T cells into tumors, using any type of T cell therapy including but not limited to any type of autologous or allogeneic T cells or genetically modified CAR T-cells. In one embodiment, poor migration and penetration of such immune cells into tumors is a roadblock reducing the effectiveness of a potentially curative therapy.

Trafficking of immune cells toward tumor foci is a dynamic process controlled by an intricate network of interactions at multiple levels. In one embodiment, the tumor microenvironment is inhospitable and inaccessible to the invasion of immune cells, because of, by way of non-limiting examples, hypoxia and low nutrients in solid tumors. Some approaches to improvement of tumor penetration have been by expression of heparinase, or targeting cancer-associated fibroblasts, the main producer of collagen within tumors, with CAR T-cells. However, challenges persist in optimizing T cell therapies.

The inventors herein discovered that by enhancing T-cell motility through downregulation of FL2, the aforementioned problems may be addressed. As will be shown in the Examples herein, exposing T cells to a FL2 inhibitor enables T-cells to migrate to and interact with small clusters or individual cancer cells in vitro. Such enhancement of migration and penetration observed in vitro will translate into enhanced tumor homing and penetration activity in vivo, and enhanced killing of tumor cells, in particular solid tumors. Such downregulation of FL2 can be achieved in vivo by exposing T cells in vivo to an inhibitor of FL2; ex vivo or in vitro methods can be achieved by exposing T cells outside the body to the inhibitor then infusing into the subject. Such therapy can also be applied in vivo at sites of T cell activation such as lymph nodes. These and other aspects of the invention are described in more detail below.

As will be described below, any of the methods or uses described herein of FL2 inhibition are equally applicable to fidgetin and fidgetin-like 1 inhibition.

### T-Cell Therapies

The invention herein is applicable to both endogenous T cells and those administered exogenously (i.e., adoptive T-cell therapies), such as but not limited to autologous T cells, CAR-T cells, tumor infiltrating lymphocytes, engineered T cell receptor lymphocytes, macrophages, microglia or natural killer cells. Other sources of T cells useful for T cell therapy are fully embraced herein, such as but not limited to donor T cells, off-the-shelf T cells, and T cell lines. In certain embodiments, the T cells are HLA matched to the patient.

Enhancing the migration to and penetration into tumors of endogenous T-cell may be achieved by administering one or more agents that inhibit FL2 parenterally or at the tumor site or in or near one or more lymph nodes proximal or even distal to the tumor site. Parenteral administering may comprise administering the agent intravenously, intra-arterially, intraperitoneally, intranasally, intramuscularly, intradermally or subcutaneously, or via guided injection, e.g., CT-guided injection, at or near a tumor site or lymph node site.

Adoptive T cells therapies comprise the administration of T cells from an exogenous source, which may be the patient's own T cells expanded and/or engineered ex vivo, or T cells from a cell line or other donor or source. Thus, the T cells may be allogeneic. In one embodiment, the allogeneic T cells are HLA matched to the patient.

Other sources of T cells include lymphoid progenitors or pluripotent stem cells.

Autologous T cells. In one embodiment (other than CAR as described elsewhere herein) comprise the T cell therapy. A patient's T cells (obtained from blood or TILs from a tumor biopsy) may be exposed ex vivo to the cancer antigen and optionally to other factors to expand the population of T cells specific for the patient's tumor. The T cells are also treated ex vivo with an agent to inhibit FL2 activity before infusion. The expanded T cells are then infused into the patient. Non-limiting examples of autologous T cell therapies include those directed to cancer antigens (with exemplary embodiments of target cancers): EBV LMP1/LMP2/EBNA1 (nasopharyngeal carcinoma), gp100 (melanoma), HBV antigens (hepatocellular carcinoma), HPV16 E6 (cervical, head and neck cancer), HPV16 E7 (cervical, vaginal, oropharyngeal cancer), MAGE-A3/4/6/10 (bladder, esophageal, lung, head and neck, NSCLC cancer), MelanA/MART-1 (melanoma), NY-ESO-1 (esophageal, melanoma, NSCLC, ovarian, synovial sarcoma), TGFβRII frameshift mutant (colorectal), and WT1 (acute myeloid leukemia).

In the practice of the invention, an agent that inhibits or knocks down FL2 levels in T cells can be exposed to autologous T cells during any ex vivo processing steps in the isolation, expansion, or other steps carried out before the cells are infused back into the patient. The exposure to the FL2 agent can occur during one or more of these processing steps.

Autologous T therapies may also include co-administration of other drugs such as but not limited to cytokines (such as IL-2 and IL-15); anti-cytokine therapies and other agents to treat cytokine release syndrome; immune checkpoint inhibitors such as ipilimumab, nivolumab, pembrolizumab, atezolizumab, avelumab, durvalumab and cemiplimab-rwlc, as well as an agent that blocks PD-1, PD-L1, CTLA-4, CTLA-4 receptor, PD1-L2, 4-1BB, OX40, LAG-3 or TIM-3. In other embodiment, other immune checkpoint inhibitors are used. Ex vivo use of checkpoint inhibition is described in Kumar et al., 2017, Onco Targets and Therapies 10:3453-3465. Small-molecular antagonists that block biochemical pathways crucial for tumor growth may also be administered.

CAR-T cells are T cells in which are engineered to express a chimeric antigen receptor (CAR) that comprises T cell activating functions and antigen-binding functions, to target the T cells to recognize tumor antigens. T cells from the patient (autologous) or from a donor (allogeneic) are isolated and expanded, then engineered to express the CAR by transducing the CAR gene. In one embodiment, the antigen of the CAR is that of a solid tumor. Some example of clinically tested CAR-T cells including those that target CD19 or CD20 (B cell antigens for B cell cancers), a combination of multiple co-stimulatory domains such as CD28 and 41BB and CD28 and OX40. Other versions include production of cytokines and co-stimulatory ligands. The CAR-T process is known to those of skill in the art.

Non-limiting examples of CAR-T cell therapies include the following antigens (with exemplary embodiments of target cancers): EBV LMP1/LMP2/EBNA1 (nasopharyngeal carcinoma), gp100 (melanoma), HBV antigens (hepatocellular carcinoma), HPV16 E6 (cervical, head and neck cancer), HPV16 E7 (cervical, vaginal, oropharyngeal cancer), MAGE-A3/4/6/10 (bladder, esophageal, lung, head and neck, NSCLC cancer), MelanA/MART-1 (melanoma), NY-ESO-1 (esophageal, melanoma, NSCLC, ovarian, synovial sarcoma), TGFβRII frameshift mutant (colorectal), and WT1 (acute myeloid leukemia). Selection of targets may be guided by following the art, such as described in Liu et al., 2019, Am J Cancer Res 9(2):228-241, incorporated herein by reference.

In the practice of the invention, an agent that inhibits or knocks down FL2 levels in T cells can be exposed to T cells during the ex vivo processing steps in the isolation, expansion, transduction, or other steps before the CAR-T cells are infused into the patient. The exposure to the FL2 agent can occur during one or more of these processing steps.

CAR-T therapies may also include co-administration of other drugs such as but not limited to cytokines (such as IL-2 and IL-15); anti-cytokine therapies and other agents to treat cytokine release syndrome; immune checkpoint inhibitors such as but not limited to ipilimumab (YERVOY), nivolumab (OPDIVO), pembrolizumab (KEYTRUDA), atezolizumab (TECENTRIQ), avelumab (BAVENCIO), durvalumab (IMFINZI) and cemiplimab-rwlc (LIBTAYO), as well as an agent that blocks PD-1, PD-L1, CTLA-4, CTLA-4 receptor, PD1-L2, 4-1BB, OX40, LAG-3 or TIM-3. In other embodiment, other immune checkpoint inhibitors are used. Ex vivo use of checkpoint inhibition is described in Kumar et al., 2017, Onco Targets and Therapies 10:3453-3465. Small-molecular antagonists that block biochemical pathways crucial for tumor growth may also be administered.

CAR-T therapies as embodied herein include any variation on the CAR-T methods, such as but no limited to second, third, fourth and beyond generation therapies. Guidance on the use of immune checkpoint inhibition and CAR-T therapies may be found in Wang et al., 2019, J. Hematol Oncol 12:59, incorporated herein by reference.

In other embodiments, CAR-T and other engineered lymphocyte therapies may be applied to any of the sources of T cells described herein, such as but not limited to lymphoid precursors, pluripotent stem cells, among others.

Tumor infiltrating lymphocytes are white blood cells that have left the bloodstream and migrated towards a tumor. They include T cells and B cells and are part of the larger category of ‘tumor-infiltrating immune cells’ which consist of both mononuclear and polymorphonuclear immune cells, (i.e., T cells, B cells, natural killer cells, macrophages, neutrophils, dendritic cells, mast cells, eosinophils, basophils, etc.) in variable proportions. TILs can often be found in the tumor stroma and within the tumor itself.

TILs can be the source of T cells for the methods described herein. In adoptive T cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. In one embodiment, TILs are expanded over the course of a few weeks with a high dose of IL-2 in 24-well plates. Selected TIL lines that presented best tumor reactivity are then further expanded in a “rapid expansion protocol” (REP), which uses anti-CD3 activation for a typical period of two weeks. The final post-REP TIL is infused back into the patient. Enhancement of the tumor homing and penetration by FL2 inhibition may be applied during one or more of the ex vivo steps described here. As noted above, therapy with other agents including immune checkpoint inhibitor therapy may accompany TIL therapy.

In one embodiment, the process can also involve a preliminary chemotherapy regimen to deplete endogenous lymphocytes in order to provide the adoptively transferred and FL2 inhibited TILs with enough access to surround the tumor sites. In one embodiment, this chemotherapy regimen is given 7 days before the expanded TIL infusion. This involves pretreatment of the patient with, in one embodiment, a combination of fludarabine and cyclophosphamide. Lympho-depletion is thought to eliminate the negative effects of other lymphocytes that may compete for growth factors and decrease anti-tumor effects of the TILs, depleting regulatory or inhibitory lymphocyte populations.

Engineered T cell receptor lymphocytes (other than CAR-T) are cells with engineered T cell receptors (TCRs), other than CAR. Non-limiting examples of those already in oncology clinical trials for cancer therapy include cancer antigens (and cancers): EBV LMP1/LMP2/EBNA1 (nasopharyngeal carcinoma), gp100 (melanoma), HBV antigens (hepatocellular carcinoma), HPV16 E6 (cervical, head and neck), HPV16 E7 (cervical, vaginal, oropharyngeal), MAGE-A3/4/6/10 (bladder, esophageal, lung, head and neck, NSCLC), MelanA/MART-1 (melanoma), NY-ESO-1 (esophageal, melanoma, NSCLC, ovarian, synovial sarcoma), TGFβRII frameshift mutant (colorectal), and WT1 (acute myeloid leukemia). Other examples are described in Paucek et al., Trends in Immunology 2019 Apr. 1; 40(4):292-309, incorporated herein by reference.

In the practice of the invention, an agent that inhibits or knocks down FL2 levels in T cells can be exposed to T cells during the ex vivo processing steps in the isolation, expansion, transduction, or other steps before the engineered cells are infused into the patient. The exposure to the FL2 agent can occur during one or more of these processing steps.

As noted above, therapy with other agents including immune checkpoint inhibitor therapy may accompany such therapy.

Other T cell sources. Other sources of T cells for T cell engineering and adoptive immunotherapy that may be used in the practice of the present methods are described in Themeli et al., Cell Stem Cell 2015 Apr. 2; 16(4):357-366, incorporated herein by reference.

Macrophages are a type of white blood cell, of the immune system, that engulfs and digests cellular debris, foreign substances, microbes, cancer cells, and anything else that does not have the type of proteins specific to healthy body cells on its surface in a process called phagocytosis. In one embodiment, the homing and/or tumor penetrating activity of macrophages is enhanced by FL2 inhibition as described herein.

Microglia are the resident mononuclear macrophages of the CNS, and constitute ˜5-20% of all glial cells in the CNS parenchyma. In one embodiment, the homing and/or tumor penetrating activity of microglia is enhanced by FL2 inhibition as described herein.

Natural killer (NK) cells are a type of cytotoxic lymphocyte critical to the innate immune system. The role NK cells play is analogous to that of cytotoxic T cells in the vertebrate adaptive immune response. NK cells provide rapid responses to virus-infected cells, acting at around 3 days after infection, and respond to tumor formation. Typically, immune cells detect the major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

Since NK cells recognize target cells when they express non-self HLA antigens (but not self), autologous (patients' own) NK cell infusions have not shown any antitumor effects. Instead, investigators are working on using allogeneic cells from peripheral blood, which requires that all T cells be removed before infusion into the patients to remove the risk of graft versus host disease, which can be fatal. This can be achieved using an immunomagnetic column (CliniMACS). In addition, because of the limited number of NK cells in blood (only 10% of lymphocytes are NK cells), their number needs to be expanded in culture. This can take a few weeks and the yield is donor-dependent. A simpler way to obtain high numbers of pure NK cells is to expand NK-92 cells whose cells continuously grow in culture and can be expanded to clinical grade numbers in bags or bioreactors (Granzin et al., Shaping of Natural Killer Cell Antitumor Activity by Ex Vivo Cultivation, Front Immunol. 2017; 8:458). Clinical studies have shown it to be well tolerated and some antitumor responses have been seen in patients with lung cancer, melanoma, and lymphoma.

In one embodiment, the homing and/or tumor penetrating activity of NK cells is enhanced by FL2 inhibition as described herein, during any one of more steps in the isolation, purification, expansion, or other ex vivo steps before infusion of the NK cells into the subject.

Lymphoid progenitors. In one embodiment, lymphoid progenitors provide a source of off-the-shelf T cells which do not require strict histocompatibility matching between donor and recipient. Moreover. while T cells can cause GVHD, their precursors do not, as they undergo positive and negative selection in the recipient's thymus. Taking advantage of this requires the ability to expand T cell precursors in culture, which is now possible due to advances in understanding T cell development. T cell precursors lack the ability to initiate GVH reactions because they complete their differentiation in the recipient's thymus wherein they become restricted to host MHC and yield T lymphocytes that are host tolerant. When transduced with a CAR, allogeneic lymphoid progenitors yield tumor-targeted T cells without causing GVHD. The main advantage of using T cell precursors for immunotherapy is that this approach does not require strict histocompatibility between donors and recipients.

Pluripotent stem cells. In another embodiment, pluripotent stem cells, which can give rise to a variety of somatic cells, may be used to supply therapeutic T cells for the purposes herein, and which can be further enhanced by the FL2 inhibition described herein. The development of cellular therapeutics relying on functionally validated, banked, broadly histocompatible cell types would have a major impact on the availability of adoptive T cell therapies. Pluripotent stem cells can give rise to a variety of somatic cells and thus have in principle the potential to serve as an endless supply of therapeutic T lymphocytes.

For any of the foregoing T cell therapies or other cell therapies wherein increased homing and/or penetration of the T cell or other cell type to or into a tumor is desirable, cells are exposed to an FL2 inhibitor as described elsewhere herein, for a sufficient time, concentration, dose, or other exposure protocol in order to enhance the tumor homing and/or tumor penetration ability of the so-treated T cells or other cell types. As noted herein, treatment of cells with a checkpoint inhibitor may also be performed.

### FL2 Inhibitors

As noted herein, increased homing to or penetration into tumors of T cells may be achieved by inhibiting FL2 expression or activity in the T cells, during in vitro or ex vivo preparation, or after infusion into the patient, or both. In one embodiment, FL2 inhibition is provided during in vitro preparation of T cells. In one embodiment, FL2 inhibition is provided during ex vivo preparation of T cells. In one embodiment, FL2 inhibition is provided in vivo to enhance endogenous T cell activity. In one embodiment, FL2 inhibition is provided during in vitro preparation of T cells and also administered in vivo after infusion of the T cells. In one embodiment, FL2 inhibition is provided during ex vivo preparation of T cells and also administered in vivo after infusion of the T cells.

Non-limiting examples of inhibitors of FL2 include aptamers, nucleic acids, oligonucleotides, and small molecules (of 2000 Daltons or less). In one embodiment, agents that inhibit FL2 expression or activity include nucleic acids such as but not limited to RNA interference agents. Non-limiting examples of RNA interference agents include shRNA and siRNA.

In one embodiment, the RNA interference agent is an siRNA (small interfering RNA). In an embodiment, the siRNA as used in the methods or compositions described herein comprises a portion which is complementary to an mRNA sequence encoding a fidgetin-like 2 protein. In an embodiment, the fidgetin-like 2 protein is a human fidgetin-like 2 protein. In an embodiment, the mRNA is encoded by the DNA sequence NCBI Reference Sequence: NM-001013690.4 (SEQ ID NO:19), and the siRNA is effective to inhibit expression of fidgetin-like 2 protein. In an embodiment, the fidgetin-like 2 protein comprises consecutive amino acid residues having the sequence set forth in SEQ ID NO:20 shown below.

In one embodiment, the siRNA comprises a double-stranded portion (duplex). In an embodiment, the siRNA is 20-25 nucleotides in length. In an embodiment the siRNA comprises a 19-21 core RNA duplex with a one or two nucleotide 3′ overhang on, independently, either one or both strands.

SiRNA oligonucleotides may be modified to enhance their activity and reduce degradation, such as described in Chakraborty et al., 2017, Mol Ther Nucleic Acids 8:132-143, incorporated herein by reference, among other teachings in the art. In one embodiment, the siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding fidgetin-like 2 protein. In an embodiment, the RNA transcript of a gene encoding fidgetin-like 2 protein is an mRNA. In an embodiment, the fidgetin-like 2 protein is a human fidgetin-like 2 protein. In an embodiment, a siRNA of the invention comprises a double-stranded RNA wherein one strand of the RNA comprises a portion having a sequence the same as a portion of 18-25 consecutive nucleotides of an RNA transcript of a gene encoding fidgetin-like 2 protein. In an embodiment, the fidgetin-like 2 protein is a human fidgetin-like 2 protein. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention comprises a 3′ overhang. In another embodiment, an siRNA of the invention comprises a 5′ overhang. In another embodiment, an siRNA of the invention comprises at least one 2′-sugar modification, such as but not limited to 2′ azido-2′deoxycytidine ribonucleic acid, 2′-azido-2′deoxyuridine ribonucleic acid, 2′-azido-2′deoxyadenosine ribonucleic acid, 2′-azido-2′-deoxyguanosine ribonucleic acid, 2′-fluoro-2′-deoxyadenosine ribonucleic acid, 2′-fluoro-2′-deoxycytidine ribonucleic acid, 2′-fluoro-2′-deoxyuridine ribonucleic acid, 2-fluorothymidine ribonucleic acid, 2′-O-methyladenosine ribonucleic acid, 2′-O-methylcytidine ribonucleic acid, 2′-O-methylguanosine ribonucleic acid, 2′-O-methyluridine ribonucleic acid, In another embodiment, an siRNA of the invention comprises at least one nucleic acid base modification, such as but not limited. to 2′-fluorodeoxy cytidine ribonucleic acid, 2′-fluorodeoxy uracil ribonucleic acid, 2′-O-methyl adenosine ribonucleic acid. Other nucleotide modifications are described in Chiu et al., 2003, RNA 9(9):1034-1048, and Peacock et al., 2011, J Org Chem 76(18):7295-7300, incorporated herein by reference.

In another embodiment, an siRNA of the invention comprises at least one phosphate backbone modification. In another embodiment, an siRNA of the invention comprises at least one 5′ phosphorylation. As used herein, “at least one” means one or more.

In one embodiment, RNAi inhibition of fidgetin-like 2 protein is effected by a short hairpin RNA (“shRNA”). The shRNA is introduced into the appropriate cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene/mRNA, in the present case the mRNA encodes fidgetin-like 2 protein. In an embodiment the fidgetin-like 2 protein is a human fidgetin-like 2 protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In one non-limiting example, a shRNA useful for the purposes herein comprises CACCGCTGGAGCCCTTTGACAAGTTCTCGAGAACTTGTCAAAGGGCTCCAGCTTTT (SEQ ID NO:23). In one embodiment, the shRNA sequence consists of CACCGCTGGAGCCCTTTGACAAGTTCTCGAGAACTTGTCAAAGGGCTCCAGCTTTT (SEQ ID NO:23).

The NCBI Reference Sequence: NM-001013690.4 (nucleic acid encoding Human fidgetin-like 2) is:

| (SEQ ID NO: 19) | |
|---|---|
| 1 | agtgagctat ggggacacta ctgcactgta gcctgggcaa cagagcaaga ccttgtctca |
| 61 | aaaatgtata tatattttgg gctttttttc ctaaaacggg aactacaaca gcatatttgc |
| 121 | gagctgatga gagtgaccca gcagagaggg aaatggatca gctctgttga agatgcactg |
| 181 | gacaccagaa cacgcccagc ccctcaacca gtggccagag cagcacctgg acgtctcctc |
| 241 | caccaccccg tcgccggccc acaagttgga gttgccccct gggggtcgcc aacgctgcca |
| 301 | ctacgcttgg gcacacgacg acatctcagc cctcactgcc tccaacctcc taaagcgcta |
| 361 | tgcagagaag tactctgggg tcttggattc tccctacgag cgtccggccc tgggcgggta |
| 421 | cagcgacgcc tccttcctca acggcgccaa aggggatccc gagccctggc cagggccgga |
| 481 | gccaccctac cccttggcct cactccacga aggcctccca ggaaccaaat cgggcggtgg |
| 541 | cggcggttcc ggggccctgg ggggctcccc agttttagcc gggaacctcc ctgaacccct |
| 601 | ctacgccggc aatgcgtgcg ggggcccatc ggcggcgccc gagtacgcgg ccggctacgg |
| 661 | cggggggtac ctggcgccgg gttactgcgc gcagacgggc gccgcgctgc ccccgccgcc |
| 721 | cccggccgcg ctcctgcagc ccccaccgcc tccggggtac gggccctcag cgccgctgta |
| 781 | caactatccc gcagggggct acgcagcgca gcccggctat ggcgcgctcc cgccgccccc |
| 841 | aggcccaccc ccggccccct acctgacccc gggcctgccc gcgcccacgc ccctgcccgc |
| 901 | gccggcaccg cccaccgcct atggcttccc cacggccgcg ccgggtgccg aatccgggct |
| 961 | gtcgctgaag cgcaaggccg ccgacgaggg gcccgagggc cgctaccgca agtacgcgta |
| 1021 | cgagcccgcc aaggcccccg tggctgacgg agcctcctac cccgccgcgg acaacggcga |
| 1081 | atgtcggggc aacgggttcc gggccaagcc gccaggagcc gcggaggagg cgtcgggcaa |
| 1141 | gtacggtggc ggcgtccccc tcaaggtcct gggctccccc gtctacggcc cgcaactgga |
| 1201 | gccctttgaa aagttcccgg agcgggcccc ggctcctcgt ggggggttcg ccgtgccgtc |
| 1261 | gggggagact cccaaaggcg tggaccctgg ggccctggag ctggtgacga gcaagatggt |
| 1321 | ggactgcggg cccccggtgc agtgggcgga tgtggcgggc cagggcgcgc tcaaggcggc |
| 1381 | gctggaggag gagctggtgt ggcccctgct caggccgccc gcctacccgg gcagcctgcg |
| 1441 | cccgccgcgg accgtcctgc tctttgggcc gcggggcgcg ggcaaagcgc tgctgggccg |
| 1501 | ctgcctcgcc acgcagctgg gcgccacgct gttgcgcctg cgcggcgcga ccctggctgc |
| 1561 | gcccggcgcc gccgagggcg cgcgcctcct ccaggccgcc ttcgcggccg cgcgctgccg |
| 1621 | cccaccctcc gtactcctca tcagcgagct agaggcgctg ctccccgccc gggacgacgg |
| 1681 | cgcggcggca gggggcgcgc tgcaggtgcc gctcctggcc tgcctggacg ggggctgcgg |
| 1741 | cgcgggggct gacggcgtgc tggttgtggg caccacctcg cggcccgcgg ctctggacga |
| 1801 | ggcgacccgc cggcgcttct ctctccgctt ctacgtggcg ctgcccgaca gcccggcccg |
| 1861 | cgggcagatc ctgcagcggg cgctggccca gcagggctgc gcgctcagtg agcgggaact |
| 1921 | ggcggcgctg gtgcagggca cgcagggctt ctctgggggc gagctggggc agctgtgcca |
| 1981 | gcaggcggcg gccggggcgg gcctcccggg gctgcagcgc cccctctcct acaaggacct |
| 2041 | ggaggcggcg ctggccaagg tgggccctag ggcctctgcc aaggaactgg actcgttcgt |
| 2101 | ggagtgggac aaaatgtacg gctccggaca ctgacggcgc gcgggggagg ccgcgggagc |
| 2161 | cgcagtccct ccgtccccgc cgcctccgcg tgggagggat gtcactgact aaacccggct |
| 2221 | ggcaggggct ggagtggtga atgtgggatc ggggacagga ggggtctgcc ggtggatatt |
| 2281 | ttttttttcg tgggaaggaa aatgcttctg ccaggcagat gccatatgcg ccgtgtactc |
| 2341 | aggtttttcc tatttattgt ggactggaag ctcgccatct ccgcccggca gaccgggcag |
| 2401 | atccggcatg ggctggcacc cggggcctta agaactcctg ctctcttgcc acaacgcttt |
| 2461 | tgtctcctcg ctatctgaat ggcaccctcc ttctccctca ctctctccat cccattctct |
| 2521 | gcattctctt ggttttctct cccttttgct ttgtcgctga cacccctgcc caccccatgc |
| 2581 | tggccctgtt tctctcctgc ccctccctcc ccagctctcc atccctcacc ctctgtgctt |
| 2641 | ctgtctccat ccctggctct ccagcgtccc tggccttttg gtccctgagc tttaatgcct |
| 2701 | ttccctgcct tctgttctta tttggactgc agtggccctt tgcaggagct ctggaggccc |
| 2761 | aggggctgag gaggagggtt acccctctac ccatctgaaa cctagggtct agggggatca |
| 2821 | aggaaaaaaa gtccccaaag aaggggaatt ttttgtttgt ttttgagggg agatcccaga |
| 2881 | aatgtagctt gtttcatatt ttagtcttct tatttttgta aaatgtgtag aatttgctgt |
| 2941 | ttttcttttt cttttgacaa ctcaggaaga aactgacctc agaaagaatg ttagactttg |
| 3001 | gctgctctcc tgtgtgcccc tcacacctgc cccctccccc ccactccatc caggggacca |
| 3061 | aattctccca gacactcaaa aaatgagact tacggggaag gggagaggaa gacccagagg |
| 3121 | cctcagtgaa accccagcta ttcctggtca gaagcagaat gtattcctaa gggcttcctc |
| 3181 | cccagggccg aggcctaggc atgaatgtgg ggagtgggct gtggggtttg agagaaggga |
| 3241 | ggccttattc ctctcctgct gctccccacc ccctgcccca cccaacccct ccgctgagtg |
| 3301 | ttttctgtga agggctatcc agagttagga tgcccttgcc caattccttc ctgagaccca |
| 3361 | gaaggtaggg tgggagggcc caaatgggaa ggtgacctaa gcagaaagtc tccagaaagg |
| 3421 | tcatgtcccc tggccctgcc ttggcagagg tccccagtga cttatgctag gaggattcca |
| 3481 | tctgggtaga cagtctggcc acaaaatcag ctactggacc tcagccatct ctgctggagg |
| 3541 | ctctgaggag gagtgagcat ccctcacttg tgggggctct gtgaggaaat gtgccttccc |
| 3601 | cattcccccg gagtcctagg tctggagctc cagggctggg agagggtgag ggagatgggc |
| 3661 | aggggtgttt tctctgacct tgggggctta gtctcagtcc tgcctgaact ttccactagg |
| 3721 | cttggaaccc ttccaagaac catatttctc tccttcccac caattttccc ttgatgaggc |
| 3781 | tttagcagtt tgctcccacc acccccagcc catttcacaa ctctgatctt agtccaaagc |
| 3841 | aggggacacg cccccccacc accacttttt ctctctccca tctcagcctc ctgtgcagtt |
| 3901 | ccttgcctgc ccgtgcattt cctagagtct actgcctccc ccctggctgg gagggtgtct |
| 3961 | gggggggatc tttcaggggc cctggcaccc agggcctgtg ctggcctagg agtgctgacc |
| 4021 | agaaggctgc tctgttcccc cccacccccg ttgctttctg gccccctctt tggagccagc |
| 4081 | cacccacagg gctttggtgc ctcagaagca gtgggctgcc gggtcacagc cgcaggctgc |
| 4141 | aaaagaccct cggagggagc atggagtgag gggttctctc tcaggtgtgt atgtattggg |
| 4201 | gggtgggggt gggtggaggg tgtcagggaa gttggggtgg gatcccagcc ttcccttcaa |
| 4261 | gaggcaggga gctctgggag gtggagtccc caccgctttc tctactaggc tcctcctgtt |
| 4321 | ccccaggctt ggggagcttt gcacaaggag actgccccca gcctagtggc acctacctca |
| 4381 | tgggctctgg ggcaggtagg ggaagggcca gtccagctct ggtaatgctg gggggaggca |
| 4441 | taccaaagaa tccaggggca gggagtgggg agggtgactt ccgagctggc ctctcccctt |
| 4501 | cctctaccca gactggggct gggatcctct cctcccgctg taaccatttc tacctcattt |
| 4561 | tgctgcgtgt tgtacatgga cgtatttatc tcctgtctga cgatgctctg cagttgtggt |
| 4621 | ctgtctacct cagaagagac tgtattttaa aagaaagtat tacacagtat taaagcgatg |
| 4681 | acatgtggtt tgcaaaaaaa aaaaaaaaaa a, |

which encodes:

MHWTPEHAQPLNQWPEQHLDVSSTTPSPAHKLELPPGGRQRCHYAWAHD
DISALTASNLLKRYAEKYSGVLDSPYERPALGGYSDASFLNGAKGDPEP
WPGPEPPYPLASLHEGLPGTKSGGGGGSGALGGSPVLAGNLPEPLYAGN
ACGGPSAAPEYAAGYGGGYLAPGYCAQTGAALPPPPPAALLQPPPPPGY
GPSAPLYNYPAGGYAAQPGYGALPPPPGPPPAPYLTPGLPAPTPLPAPA
PPTAYGFPTAAPGAESGLSLKRKAADEGPEGRYRKYAYEPAKAPVADGA
SYPAADNGECRGNGFRAKPPGAAEEASGKYGGGVPLKVLGSPVYGPQLE
PFEKFPERAPAPRGGFAVPSGETPKGVDPGALELVTSKMVDCGPPVQWA
DVAGQGALKAALEEELVWPLLRPPAYPGSLRPPRTVLLFGPRGAGKALL
GRCLATQLGATLLRLRGATLAAPGAAEGARLLQAAFAAARCRPPSVLLI
SELEALLPARDDGAAAGGALQVPLLACLDGGCGAGADGVLVVGTTSRPA
ALDEATRRRFSLRFYVALPDSPARGQILQRALAQQGCALSERELAALVQ
GTQGFSGGELGQLCQQAAAGAGLPGLQRPLSYKDLEAALAKVGPRASAK
ELDSFVEWDKMYGSGH (human fidgetin-like 2; SEQ ID
NO: 20).

In one embodiment, the siRNA that inhibits FL2 comprises a sense or antisense sequence selected from the table below.

| RNA sequences | Sequence ID |
|---|---|
| UUACACAGUAUUAAAGCGAUU (sense) | SEQ ID NO: 1 |
| UCGCUUUAAUACUGUGUAAUU (antisense) | SEQ ID NO: 2 |
| CAUCUGAAACCUAGGGUCUUU (sense) | SEQ ID NO: 3 |
| AGACCCUAGGUUUCAGAUGUU (antisense) | SEQ ID NO: 4 |
| GUGACUUAUGCUAGGAGGAUU (sense) | SEQ ID NO: 5 |
| UCCUCCUAGCAUAAGUCACUU (antisense) | SEQ ID NO: 6 |
| GGUCAGAAGCAGAAUGUAUUU ( sense) | SEQ ID NO: 7 |
| AUACAUUCUGCUUCUGACCUU (antisense) | SEQ ID NO: 8 |
| CGCCGGCCCACAAGUUGGAdTdT (sense) | SEQ ID NO: 9 |
| UCCAACUUGUGGGCCGGCGdTdT (antisense) | SEQ ID NO: 10 |
| CAGCUCGAGCCCUUUGACAdTdT (sense) | SEQ ID NO: 11 |
| UGUCAAAGGGCUCGAGCUGdTdT (antisense) | SEQ ID NO: 12 |
| CCUCCAACCUCCUCAAGAGdTdT (sense) | SEQ ID NO: 13 |
| CUCUUGAGGAGGUUGGAGGdTdT (antisense) | SEQ ID NO: 14 |
| CGUUGCUGCUCAUCAGCGAdTdT (sense) | SEQ ID NO: 15 |
| UCGCUGAUGAGCAGCAACGdTdT (antisense) | SEQ ID NO: 16 |
| fUfUmA fCmAfC AGU AUU AAA GCG ATT(sense) | SEQ ID NO: 17 |
| (Phos) U CGC UUU AAU ACU G UG UAA TT (antisense) | SEQ ID NO: 18 |
| 5′-UUACACAGUAUUAAAGCGATT-3′ (sense) | SEQ ID NO: 34 |
| (Phos) 5′ - mUmCGCUUUAAUACUGUGUAATT-3′ (antisense) | SEQ ID NO: 35 |
| (Phos) 5′ - mU(s)mC(s)GCUUUAAUACUGUGUAATT-3′ (antisense) | SEQ ID NO: 36 |
| (Phos) 5′ - fUfCGCUUUAAUACUGUGUAATT -3′ (antisense) | SEQ ID NO: 37 |
| (Phos) 5′ -fU(s)fC(s)GCUUUAAUACUGUGUAATT-3′(antisense) | SEQ ID NO: 38 |
| (Phos) 5′ - mU(s)mC(s)GCUUUAAUAmCfUmGfUmGfUmAmATT-3′ | SEQ ID NO: 39 |
| (antisense) | |
| (Phos) 5′ - U(s)CGCUUUAAUACUGUGUAATT-3′ (antisense) | SEQ ID NO: 40 |
| (Phos) 5′ - mUfCmGfCmUfUmUAAfUmAfCmUGmUmGfUmAmATT | SEQ ID NO: 41 |
| (antisense) | |
| 5′ | SEQ ID NO: 42 |
| mUmUmAmCmAmCmAmGmUmAmUmUmAmAmAmGmCmGmAmUmU- | |
| 3′ (antisense) | |
| (Phos) 5′ - | SEQ ID NO: 43 |
| mUmCmGmCmUmUmUmAmAmUmAmCmUmGmUmGmUmAmAmUmU- | |
| 3′ (antisense) | |
| 5′ mUmUmAmCmAmCmAmGmUmAmUmUmAmAmAmGdCdGdATT-3′ | SEQ ID NO: 44 |
| (sense) | |
| 5′ mUmUmAmCmAmCmAmGmUmAmUmUmAmAmAmGdCmGmATT-3′ | SEQ ID NO: 45 |
| (sense) | |
| 5′ UUACACAGUAUUAAAGCGA-3′ (sense) | SEQ ID NO: 46 |
| (Phos) 5′ - U(s)CGCUUUAAUACUGUGUAATT-3′ (antisense) | SEQ ID NO: 47 |
| (Phos) 5′ - UCGCUUUAAUACUGUGUAATT-3′(antisense) | SEQ ID NO: 48 |
| (Phos) 5′ - U(s)C(s)GCUUUAAUACUGUGUAATT-3′(antisense) | SEQ ID NO: 49 |
| 5′-mUmUACACAGUAUUAAAGCGA-3′(sense) | SEQ ID NO: 50 |
| (Phos) 5′ - U(s)CGCUUUAAUACUGUGUmAmATT-3′(antisense) | SEQ ID NO: 51 |
| (Phos) 5′ - UCGCUUUAAUACUGUGUAATT-3′ (antisense) | SEQ ID NO: 52 |
| (Phos) 5′ - U(s)C(s)GCUUUAAUACUGUGUAA T(s)T-3′ (antisense) | SEQ ID NO: 53 |
| 5′ 1U1thA1C1ACAGUAUUAAAGCGATT-3′ (sense) | SEQ ID NO: 54 |
| (Phos) 5′ - UCGCUUUAAUACUG1U1G1U1A1A TT -3′ (antisense) | SEQ ID NO: 55 |
| 5′ fUfU1AfC1ACAGUAUUAAAGCGA-3′ (sense) | SEQ ID NO: 56 |
| (Phos) 5′ - mU(s)mCmGCUUUAAUACUGUGUAATT -3′ (antisense) | SEQ ID NO: 57 |

wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate. Thus, for example dT represents deoxythymidine, dC represents deoxycytidine, fC represents 2′-fluorodeoxy cytidine ribonucleic acid, fU represents 2′-fluorodeoxy uracil ribonucleic acid, mA represents 2′-O-methyl adenosine ribonucleic acid, mU represents 2′-O-methyl uracil ribonucleic acid, mC represents 2′-O-methyl cytosine ribonucleic acid, and mG represents 2′-O-methyl guanosine ribonucleic acid.

In some embodiments, the siRNA may have a 5′-phosphodiester cap, as abbreviated “(Phos)” in the aforementioned sequences. In some embodiments, the siRNA does not have a 5′-phosphodiester cap. siRNA sequences without a 5′-phosphodiester cap are fully embraced herein.

A phosphorothioate linkage between nucleotides is represented in the sequences by “(s)”.

Locked nucleotides in one embodiment comprise a ribose with a 2′-O, 4′-C methylene bridge, for example, 2′-O, 4′-C methylene adenosine (1A); 2′-O, 4′-C methylene guanosine (1G); 2′-O, 4′-C methylene cytidine (1C); 2′-O, 4′-C methylene uridine (1U); and 2′-O, 4′-C methylene thymine (1T) ribonucleosides. In other embodiments, the locked nucleic acid comprises a methyl group attached to the methylene group. Other types of locked nucleic acids are embraced herein.

In one embodiment, the FL2 siRNA is double-stranded and comprises any complementary sense sequence and antisense sequence from the foregoing table.

Non-limiting examples of such double-stranded sequences include SEQ ID NO:1 and SEQ ID NO: 2, SEQ ID NO:3 and SEQ ID NO: 4, SEQ ID NO:5 and SEQ ID NO: 6, SEQ ID NO:7 and SEQ ID NO: 8, SEQ ID NO:9 and SEQ ID NO: 10, SEQ ID NO:11 and SEQ ID NO: 12, SEQ ID NO:13 and SEQ ID NO: 14, SEQ ID NO:15 and SEQ ID NO: 16, and SEQ ID NO:17 and SEQ ID NO: 18. In some embodiments, the siRNA is single-stranded, selected from among SEQ ID NO:1-18 above.

In one embodiment, a double stranded nucleic acid is provided consisting of complementary nucleic acid molecules selected from among SEQ ID NOs: 34-57 or from among SEQ ID NOS: 1-18 or 34-57. In one embodiment, the double stranded nucleic acid comprises a sense strand and an antisense strand. In one embodiment, the double stranded nucleic acid consists of a sense strand and an antisense strand.

In one embodiment, a double stranded nucleic acid is provided consisting of a sense strand selected from SEQ ID NOs: 1, 3, 5, 7, 9, 11, 13, 15 or 17; and an antisense strand selected from SEQ ID NOs: 2, 4, 6, 8, 10, 12, 14, 16 or 18.

In one embodiment, a double stranded nucleic acid is provided consisting of a sense strand selected from SEQ ID NOs: 1, 17, 34, 42, 44, 45, 46, 50 and 54; and an antisense strand selected from SEQ ID NOs: 2, 18, 35, 36, 37, 38, 39, 40, 41, 43, 47, 48, 49, 51, 52, 53, 55 and 57.

In one embodiment, a double stranded nucleic acid is provided consisting of a sense strand selected from SEQ ID NOs: 1, 17, 34, 42, 44, 45, 46, 50, 54 and 56; and an antisense strand selected from SEQ ID NOs: 2, 4, 6, and 8.

In one embodiment, a double stranded nucleic acid is provided consisting of a sense strand selected from SEQ ID NOs: 1, 3, 5 and 7; and an antisense strand selected from SEQ ID NOs: 18, 35, 36, 37, 38, 39, 40, 41, 43, 47, 48, 49, 51, 52, 53, 55 and 57.

In one embodiment, a double stranded nucleic acid is provided consisting of a sense strand selected from SEQ ID NOs: 1, 3, 5, 7, 9, 11, 13, 15, 17, 34, 42, 44, 45, 46, 50 and 54; and an antisense strand selected from SEQ ID NOs: 2, 4, 6, 8, 10, 12, 14, 16, 18, 35, 36, 37, 38, 39, 40, 41, 43, 47, 48, 49, 51, 52, 53, 55 and 57.

In one embodiment, a double stranded nucleic acid is provided comprising a sense strand selected from SEQ ID NOs: 1, 3, 5, 7, 9, 11, 13, 15, 17, 34, 42, 44, 45, 46, 50 and 54; and an antisense strand selected from SEQ ID NOs: 2, 4, 6, 8, 10, 12, 14, 16, 18, 35, 36, 37, 38, 39, 40, 41, 43, 47, 48, 49, 51, 52, 53, 55 and 57.

In one embodiment, a double-stranded nucleic acid is provided consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; or SEQ ID NO:56 and SEQ ID NO:57.

In one embodiment, a double stranded nucleic acid is provided comprising at least one nucleic acid molecule selected from among SEQ ID NOs: 1-18 or 34-57.

In one embodiment, a double stranded nucleic acid is provided comprising two nucleic acid molecules selected from among SEQ ID NOs: 1-18 or 34-57. In one embodiment, the double stranded nucleic acid comprises a sense strand and an antisense strand.

In one embodiment, each strand of the double stranded nucleic acid has no more than 52 nucleotides.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from SEQ ID NOs: 2, 18, 35, 36, 37, 38, 39, 40, 41, 43, 47, 48, 49, 51, 52, 53, 55 and 57.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from SEQ ID NOs: 4, 6, 8, and 10.

In one embodiment, a double stranded nucleic acid is provided comprising a sense strand comprising a nucleic acid molecule selected from SEQ ID NOs: 1, 3, 5, 7 and 9; and an antisense strand comprising a nucleic acid molecule selected from SEQ ID NO: 18, 35, 36, 37, 38, 39, 40, 41, 43, 47, 48, 49, 51, 52, 53, 55 and 57.

In one embodiment, the double-stranded nucleic acid comprises nucleic acid molecules comprising SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; or SEQ ID NO:56 and SEQ ID NO:57.

In one embodiment, each strand of the double stranded nucleic acid has no more than 52 nucleotides.

In one embodiment, any one of the foregoing nucleic acids has at least one nucleotide is modified or further modified. In one embodiment, the modified nucleotide is selected from 2′-O-methyl-adenosine, 2′-O-methyl-uridine, 2′-O-methyl-cytosine, 2′-O-methyl-guanosine, 2′-O-methyl-thymidine, 2′-fluoro-adenosine, 2′-fluoro-cytidine, 2′-fluoro-guanosine, 2′-fluoro-uracil, 2′-fluoro-thymidine, deoxycytosine, deoxyguanosine, deoxyadenosine, deoxythymidine, deoxyuridine, a locked adenosine, a locked uridine, a locked guanosine, a locked cytidine, a phosphorothioate, and a phosphodiester cap. In one embodiment, at least one additional nucleotide or modified nucleotide is added to an end of the nucleic acid.

As noted above, locked nucleotides in one embodiment comprise a ribose with a 2′-O, 4′-C methylene bridge, for example, 2′-O, 4′-C methylene adenosine (1A); 2′-O, 4′-C methylene guanosine (1G); 2′-O, 4′-C methylene cytidine (1C); 2′-O, 4′-C methylene uridine (1U); and 2′-O, 4′-C methylene thymine (1T) ribonucleosides. In other embodiments, the locked nucleic acid comprises a methyl group attached to the methylene group. Other types of locked nucleic acids are embraced herein.

In other examples, a siRNA directed to FL2 may be selected from among:

SEQ ID NO: 58
5′ - fUfCGCUUUAAUACUGUGUAATT -3′ (antisense)
SEQ ID NO: 59
5′ -fU(s)fC(s)GCUUUAAUACUGUGUAATT-3′(antisense)
SEQ ID NO: 60
5′ - mU(s)mC(s)GCUUUAAUAmCfUmGfUmGfUmAmATT-3′
(antisense)
SEQ ID NO: 61
5′ - U(s)CGCUUUAAUACUGUGUAATT-3′ (antisense)
SEQ ID NO: 62
5′ - mUfCmGfCmUfUmUAAfUmAfCmUGmUmGfUmAmATT
(antisense)
SEQ ID NO: 63
5′-mUmCmGmCmUmUmUmAmAmUmAmCmUmGmUmGmUmAmAmUmU-3′
(antisense)
SEQ ID NO: 64
5′ - U(s)CGCUUUAAUACUGUGUAATT-3′ (antisense)
SEQ ID NO: 65
5′ - UCGCUUUAAUACUGUGUAATT-3′(antisense)
SEQ ID NO: 66
5′ - U(s)C(s)GCUUUAAUACUGUGUAATT-3′(antisense)
SEQ ID NO: 67
5′ - U(s)CGCUUUAAUACUGUGUmAmATT-3′(antisense)
SEQ ID NO: 68
5′ - UCGCUUUAAUACUGUGUAATT-3′ (antisense)
SEQ ID NO: 69
5′ - U(s)C(s)GCUUUAAUACUGUGUAA T(s)T-3′
(antisense)
SEQ ID NO: 70
5′ - UCGCUUUAAUACUG1U1G1U1A1A TT -3′ (antisense)
SEQ ID NO: 71
5′ - mU(s)mCmGCUUUAAUACUGUGUAATT -3′ (antisense)
SEQ ID NO: 72
5′ - U CGC UUU AAU ACU G UG UAA TT (antisense)

wherein the abbreviations are the same as described above.

In one embodiment, a double stranded nucleic acid is provided consisting of a sense strand selected from SEQ ID NOs: 1, 17, 34, 42, 44, 45, 46, 50 and 54; and an antisense strand selected from any one of SEQ ID NOs: 58-72.

In one embodiment, a double stranded nucleic acid is provided consisting of a sense strand selected from SEQ ID NOs: 1, 3, 5 and 7; and an antisense strand selected from any one of SEQ ID NOs: 58-72.

In one embodiment, a double stranded nucleic acid is provided consisting of a sense strand selected from SEQ ID NOs: 1, 3, 5, 7, 9, 11, 13, 15, 17, 34, 42, 44, 45, 46, 50 and 54; and an antisense strand selected from any one of SEQ ID NOs: 58-72.

In one embodiment, a double stranded nucleic acid is provided comprising a sense strand selected from SEQ ID NOs: 1, 3, 5, 7, 9, 11, 13, 15, 17, 34, 42, 44, 45, 46, 50 and 54; and an antisense strand selected from any one of SEQ ID NOs: 58-72.

In one embodiment, a double-stranded nucleic acid is provided consisting of SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

In one embodiment, a double stranded nucleic acid is provided comprising at least one nucleic acid molecule selected from among SEQ ID NOs: 58-72.

In one embodiment, a double stranded nucleic acid is provided comprising two nucleic acid molecules selected from among SEQ ID NOs: 1-18 or 34-72. In one embodiment, the double stranded nucleic acid comprises a sense strand and an antisense strand.

In one embodiment, each strand of the double stranded nucleic acid has no more than 52 nucleotides.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from any one of SEQ ID NOs: 58-72.

In one embodiment, a double stranded nucleic acid is provided comprising a sense strand comprising a nucleic acid molecule selected from SEQ ID NOs: 1, 3, 5, 7 and 9; and an antisense strand comprising a nucleic acid molecule selected from any one of SEQ ID NOs:58-72.

In one embodiment, the double-stranded nucleic acid comprises nucleic acid molecules comprising SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

Any of the compositions and uses of siRNA directed to FL2 as described elsewhere herein may utilize any of the foregoing single stranded nucleic acid sequences SEQ ID NOs:58-72, or a double stranded nucleic acids comprising or consisting of any of SEQ ID NOs:58-72.

Any of the nucleic acid sequences described herein may be prepared by any method known in the art, and purified by HPLC or any other method to provide inhibitors suitable for use for the in vitro, ex vivo or in vivo purposes described herein. In some embodiments, the purity of the inhibitor is equal to or greater than 85%. In some embodiment the purity is equal to or greater than 90%. In some embodiments the purity is equal to or greater than 95%. In some embodiment the purity is equal to or greater than 98%. In some embodiments the purity is equal to or greater than 99%. In some embodiments wherein the inhibitor is or comprises a duplex, the purity of the duplex is equal to or greater than 85%. In some embodiments wherein the inhibitor is or comprises a duplex, the purity of the duplex is equal to or greater than 90%. In some embodiments wherein the inhibitor is or comprises a duplex, the purity of the duplex is equal to or greater than 95%. In some embodiments wherein the inhibitor is or comprises a duplex, the purity of the duplex is equal to or greater than 98%. In some embodiments wherein the inhibitor is or comprises a duplex, the purity of the duplex is equal to or greater than 99%. In some embodiments the inhibitor is prepared under current Good Manufacturing Practices. In some embodiments the inhibitor is prepared for human use. In some embodiments the inhibitor is prepare for in vitro or ex vivo use for subsequent administration to humans. In some embodiments the inhibitor is prepared for human administration.

In one aspect, a composition is provided comprising any of the foregoing nucleic acid molecules or double-stranded nucleic acids, and a pharmaceutically acceptable carrier, vehicle, excipient or diluent.

In any of the embodiments described herein, an inhibitor of fidgetin or of fidgetin-like 1 (FL1) may be used to achieve any of the methods and results described herein for inhibitors of fidgetin-like 2 (FL2). Thus, increased homing to or penetration into tumors of T cells may be achieved by inhibiting fidgetin or fidgetin-like 1 expression or activity in the T cells, during in vitro or ex vivo preparation, or after infusion into the patient. Non-limiting examples of inhibitors of fidgetin or fidgetin-like 1 include aptamers, nucleic acids, oligonucleotides, and small molecules (of 2000 Daltons or less). In one embodiment, agents that inhibit fidgetin or fidgetin-like 1 expression or activity include nucleic acids such as but not limited to RNA interference agents. Non-limiting examples of RNA interference agents include shRNA and siRNA.

Thus, in an embodiment, the siRNA (small interfering RNA) as used in the methods or compositions described herein comprises a portion which is complementary to an mRNA sequence encoding a fidgetin protein. In an embodiment, the fidgetin protein is a human fidgetin protein. In an embodiment, the siRNA is effective to inhibit expression of fidgetin protein. In an embodiment, the fidgetin protein comprises consecutive amino acid residues having the sequence set forth in SEQ ID NO:22. In an embodiment, the siRNA is effective to inhibit expression of fidgetin protein.

In an embodiment, the siRNA that inhibits fidgetin comprises a double-stranded portion (duplex). In an embodiment, the siRNA is 20-25 nucleotides in length. In an embodiment the siRNA comprises a 19-21 core RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated. Any of the modifications described herein regarding FL2 are further embodiments of the fidgetin inhibitor.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding fidgetin protein. In an embodiment, the RNA transcript of a gene encoding fidgetin protein is an mRNA. In an embodiment, the fidgetin protein is a human fidgetin protein. In an embodiment, a siRNA of the invention comprises a double-stranded RNA wherein one strand of the RNA comprises a portion having a sequence the same as a portion of 18-25 consecutive nucleotides of an RNA transcript of a gene encoding fidgetin protein. In an embodiment, the fidgetin protein is a human fidgetin protein. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention inhibiting fidgetin is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention inhibiting fidgetin comprises at least one 2′-sugar modification. In another embodiment, an siRNA of the invention comprises at least one nucleic acid base modification. In another embodiment, an siRNA of the invention comprises at least one phosphate backbone modification. As used herein, “at least one” means one or more.

In one embodiment, RNAi inhibition of fidgetin protein is effected by a short hairpin RNA (“shRNA”). The shRNA is introduced into the appropriate cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene/mRNA, in the present case the mRNA encodes fidgetin protein. In an embodiment the fidgetin protein is a human fidgetin protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In an embodiment, the fidgetin is encoded by a nucleic acid sequence comprising the following:

| (SEQ ID NO: 21) | |
|---|---|
| 1 | gggtttgaaa ttccaacatg gcagaggctg cagtccgtct tcccttcaaa aacttggaat |
| 61 | gatttcaaat cataggcacc ttcacttaac cctagcttcc attcatcagc aaacacatcg |
| 121 | gatcgatgct acgctaacct atcgggttct ctctccgcgc gttcaggtta aatgaatacc |
| 181 | tgacgaaagg gcccacgttt caaggcagtg acatttgata gctgagagga aaagtggctt |
| 241 | taatgaaaag caacctttgg aattcctgct tgtgagaaat ccaattcagc tttttgtgct |
| 301 | gccagcaaga aatgatcagt agcaccagtg tttatggctt gaagatgcag tggacgccag |
| 361 | agcatgccca gtggccagaa cagcactttg acatcacctc aaccactcgg tctcctgccc |
| 421 | acaaagttga agcctacaga ggtcatctgc agcgcaccta tcagtacgcc tgggcgaatg |
| 481 | atgacatatc tgctctgact gcatccaacc tactaaaaaa atatgcagag aagtattccg |
| 541 | gcattttgga aggtcctgtg gaccgacccg tactcagcaa ctattcggac acaccatcag |
| 601 | gactagtgaa cggtcggaaa aatgaaagtg aaccctggca gccttccttg aattcagaag |
| 661 | ctgtttatcc catgaactgt gttccggatg ttatcactgc cagcaaagct ggagtcagtt |
| 721 | cagccctccc tccagcagat gtctctgcga gtataggaag ctctcctggg gtagccagca |
| 781 | acctgacaga acctagttat tcaagtagta cctgtggaag ccacactgta cccagtcttc |
| 841 | atgcagggct cccatctcag gaatatgccc caggatacaa cggatcatat ttgcattcta |
| 901 | cttatagtag ccagccagca cctgcacttc cttcacctca tccgtctcct ttgcatagct |
| 961 | ctgggctact acagccccca ccaccacctc ctccgccacc agccttggtc ccaggctaca |
| 1021 | atgggacttc taacctctcc agttacagct atccgtctgc tagctatcct cctcagactg |
| 1081 | ctgtggggtc tgggtacagc cctggggggg caccgcctcc gccttcagcg tacctgcctt |
| 1141 | caggaattcc tgctcccacc cccctacccc ccaccactgt tcctggctac acctaccagg |
| 1201 | gccatggttt gacacctatt gcaccgtcgg ctctgacaaa cagttcagca agttctctca |
| 1261 | aaaggaaagc tttctacatg gcagggcaag gagatatgga ctccagttat ggaaattaca |
| 1321 | gctatggcca acagagatct acacagagtc ctatgtacag aatgcccgac aacagcattt |
| 1381 | caaacacaaa tcgggggaat ggctttgaca gaagtgctga aacatcatcc ttagcattta |
| 1441 | agccaacgaa gcagctaatg tcctctgaac agcaaaggaa attcagcagc cagtccagta |
| 1501 | gggctctgac ccctccttcc tacagtactg ctaaaaattc attgggatca agatccagtg |
| 1561 | aatcctttgg gaagtacaca tcgccagtaa tgagtgagca tggggacgag cacaggcagc |
| 1621 | tcctctctca cccaatgcaa ggccctggac tccgtgcagc tacctcatcc aaccactctg |
| 1681 | tggacgagca actgaagaat actgacacgc acctcatcga cctggtaacc aatgagatta |
| 1741 | tcacccaagg acctccagtg gactggaatg acattgctgg tctcgacctg gtgaaggctg |
| 1801 | tcattaaaga ggaggtttta tggccagtgt tgaggtcaga cgcgttcagt ggactgacgg |
| 1861 | ccttacctcg gagcatcctt ttatttggac ctcgggggac aggcaaaaca ttattgggca |
| 1921 | gatgcatcgc tagtcagctg ggggccacat ttttcaaaat tgccggttct ggactagtcg |
| 1981 | ccaagtggtt aggagaagca gagaaaatta tccatgcctc ttttcttgtg gccaggtgtc |
| 2041 | gccagccctc ggtgattttt gttagtgaca ttgacatgct tctctcctct caagtgaatg |
| 2101 | aggaacatag tccagtcagt cggatgagaa ccgaatttct gatgcaactg gacactgtac |
| 2161 | taacttcggc tgaggaccaa atcgtagtaa tttgtgccac cagtaaacca gaagaaatag |
| 2221 | atgaatccct tcggaggtac ttcatgaaac gacttttaat cccacttcct gacagcacag |
| 2281 | cgaggcacca gataatagta caactgctct cacagcacaa ttactgtctc aatgacaagg |
| 2341 | agtttgcact gctcgtccag cgcacagaag gcttttctgg actagatgtg gctcatttgt |
| 2401 | gtcaggaagc agtggtgggc cccctccatg ccatgccagc cacagacctt tcagccatta |
| 2461 | tgcccagcca gttgaggccc gttacatatc aagactttga aaatgctttc tgcaagattc |
| 2521 | agcctagcat atctcaaaag gagcttgata tgtatgttga atggaacaaa atgtttggtt |
| 2581 | gcagtcagtg ataacttctt tagaaaaaaa aaatgtaatg aatgttggca cacacacata |
| 2641 | aaacctgcta catagggaat agagcccctt tecagtagag tttaaattgc aaagggtact |
| 2701 | ggggaagatg acgattaagt tgcatcttta gagtcagggt agatttggag gaaaagtgca |
| 2761 | tcaaatgaga gcttctgatt tgaaagcccc agatgacaga aagcatatgt ggatgctcag |
| 2821 | ttctgttcaa gctagacaac actcaccaag gagcaaggtg caagtgtgtt gatttcagaa |
| 2881 | ggacatgaac ctcgtgtgtt gattccattc tgctgttctc gagatttagt tgctgtcaag |
| 2941 | tgcctggagt ggtgctttat tttttgtttg cctcacaatt acattggtgg catgtgctaa |
| 3001 | tataaagagc tttaacttca aacattattg gactaaagag atgaacagtt gtgttatgac |
| 3061 | agaaaaccag atttttgcca ttttaagagc aacagtattc ctcaatcctg tctgttctgc |
| 3121 | agtattaagc taagaacagg taaaacaggg taacggtaat ctggacctta atttctgcag |
| 3181 | ttcatttctt ttaatgttct tgtctgcaaa aactcaggaa agtgattgtg atttgtacag |
| 3241 | tacctcaaag gaatgtgttg aaagcactat gtactgctga gagtaatagg ataggcttca |
| 3301 | atgttacttt atattaaaat gtatgtttac ctcaacaatt ggaaaatagc aaggaaaatt |
| 3361 | actttgaatg tatccagaaa aatactgaag tgtgatacaa ctgaatattt acagtttaaa |
| 3421 | gtagaaatgg aaggattttt ttaagttctt ttactaatta tggggaatta accagagcag |
| 3481 | aataattctt tatgtcaata actgcaagag ttcttagtac attgctcctt gataattaag |
| 3541 | tgaaaatgtt cttaaaaggt acactggtta attgaaagct acttattcag tttgtgttag |
| 3601 | tgtctagacc tgtcagccac aagacctgtt taggaccctg aaagtcacag tacctaaaaa |
| 3661 | ctatgactgc ctttttattg cataggtggt agtggtggtg atggtggtgg tagtttgcaa |
| 3721 | gttatctctt aaaactgctg ggaatggtgt cattctattc actaatctag cttatagact |
| 3781 | tgccgtgctg tttgatagaa tgcagaggat agcaaccaaa acaaatacac aaataaataa |
| 3841 | aaacaaaaac caaccaacaa accaacttac atacacatat atatatccac aaagaacctc |
| 3901 | tccatctcct ccccttcttt ttgactccac tcttgtcagt gcaattttgc ttctcatttt |
| 3961 | gaaatctggg ctgtagtgct cctgctttat ttctacctca gttttgttac atttctcttg |
| 4021 | gaaagtaaag tagaaaattg gaagtggaca cacacactgc aatgtagctt gccaaacatg |
| 4081 | ttactttgtt ttcttccatc tttcaccgta aatctagttt ccaaagacat cagcatttgt |
| 4141 | gcttacttcc acctcagtct accagcccca cccctaccca tggcataagt ggcatttttc |
| 4201 | ttaatttcct atttttctcc tgctctctgt caagttgttc tttgtatcct ttaatgcttt |
| 4261 | atgtgcaacc tttcattgat agtgggctga tgtttggcaa tgcttctgaa ctgtcacaga |
| 4321 | gcaggctgta gctttccaca gccactgccc atgcataagc agaacagcct ggccttttga |
| 4381 | atgtattttc ctgggttttt tccccttttc tttttttagt ttagagatgc agtaacaaaa |
| 4441 | ctgttgcaaa gcactggcat tttatgtatt caataaataa gtgatgtaca tttttaaaaa |
| 4501 | aatttaaata aatgcaatga gaagccccaa gaaag. |

The sequence of human fidgetin protein is:

(SEQ ID NO: 22)

| | |
|---|---|
| 1 | MISSTSVYGL KMQWTPEHAQ WPEQHFDITS TTRSPAHKVE |
| | AYRGHLQRTY QYAWANDDIS |
| 61 | ALTASNLLKK YAEKYSGILE GPVDRPVLSN YSDTPSGLVN |
| | GRKNESEPWQ PSLNSEAVYP |
| 121 | MNCVPDVITA SKAGVSSALP PADVSASIGS SPGVASNLTE |
| | PSYSSSTCGS HTVPSLHAGL |
| 181 | PSQEYAPGYN GSYLHSTYSS QPAPALPSPH PSPLHSSGLL |
| | QPPPPPPPPP ALVPGYNGTS |
| 241 | NLSSYSYPSA SYPPQTAVGS GYSPGGAPPP PSAYLPSGIP |
| | APTPLPPTTV PGYTYQGHGL |
| 301 | TPIAPSALTN SSASSLKRKA FYMAGQGDMD SSYGNYSYGQ |
| | QRSTQSPMYR MPDNSISNTN |
| 361 | RGNGFDRSAE TSSLAFKPTK QLMSSEQQRK FSSQSSRALT |
| | PPSYSTAKNS LGSRSSESFG |
| 421 | KYTSPVMSEH GDEHRQLLSH PMQGPGLRAA TSSNHSVDEQ |
| | LKNTDTHLID LVTNEIITQG |
| 481 | PPVDWNDIAG LDLVKAVIKE EVLWPVLRSD AFSGLTALPR |
| | SILLFGPRGT GKTLLGRCIA |
| 541 | SQLGATFFKI AGSGLVAKWL GEAEKIIHAS FLVARCRQPS |
| | VIFVSDIDML LSSQVNEEHS |
| 601 | PVSRMRTEFL MQLDTVLTSA EDQIVVICAT SKPEEIDESL |
| | RRYFMKRLLI PLPDSTARHQ |
| 661 | IIVQLLSQHN YCLNDKEFAL LVQRTEGFSG LDVAHLCQEA |
| | VVGPLHAMPA TDLSAIMPSQ |
| 721 | LRPVTYQDFE NAFCKIQPSI SQKELDMYVE WNKMFGCSQ. |

Non-limiting examples of siRNA sequences that inhibit fidgetin include:

(SEQ ID NO: 24)
ACUAGUGAACGGUCGGAAA,
(SEQ ID NO: 25)
GCAGAGAAGUAUUCCGGCA,
(SEQ ID NO: 26)
GGCCAUGGUUUGACACCUA
and
(SEQ ID NO: 27)
AGUUGAGGCCCGUUACAUA.

Any of the foregoing sequence may be provided as a double stranded nucleic acid with a complementary sequence. Such siRNA molecules may be provided in any of the compositions described herein and used for any of the purposes described herein for FL2.

The nucleic acid sequences described herein may be prepared by any method known in the art, and purified by HPLC or any other method to provide inhibitors suitable for use for the in vitro, ex vivo or in vivo purposes described herein. In some embodiments, the purity of the inhibitor is equal to or greater than 85%. In some embodiment the purity is equal to or greater than 90%. In some embodiments the purity is equal to or greater than 95%. In some embodiment the purity is equal to or greater than 98%. In some embodiments the purity is equal to or greater than 99%. In some embodiments wherein the inhibitor is or comprises a duplex, the purity of the duplex is equal to or greater than 85%. In some embodiments wherein the inhibitor is or comprises a duplex, the purity of the duplex is equal to or greater than 90%. In some embodiments wherein the inhibitor is or comprises a duplex, the purity of the duplex is equal to or greater than 95%. In some embodiments wherein the inhibitor is or comprises a duplex, the purity of the duplex is equal to or greater than 98%. In some embodiments wherein the inhibitor is or comprises a duplex, the purity of the duplex is equal to or greater than 99%. In some embodiments the inhibitor is prepared under current Good Manufacturing Practices. In some embodiments the inhibitor is prepared for human use. In some embodiments the inhibitor is prepare for in vitro or ex vivo use for subsequent administration to humans. In some embodiments the inhibitor is prepared for human administration.

In any of the embodiments described herein, an inhibitor of fidgetin-like 1 (FL1) may be used to carry out any of the methods and achieve the results described herein for fidgetin-like 2 (FL2). Thus, increased homing to or penetration into tumors of T cells may be achieved by inhibiting fidgetin-like 1 expression or activity in the T cells, during in vitro or ex vivo preparation, or after infusion into the patient. Non-limiting examples of inhibitors of fidgetin-like 1 include aptamers, nucleic acids, oligonucleotides, and small molecules (of 2000 Daltons or less). In one embodiment, agents that inhibit fidgetin-like 1 expression or activity include nucleic acids such as but not limited to RNA interference agents. Non-limiting examples of RNA interference agents include shRNA and siRNA.

Thus, in an embodiment, the siRNA (small interfering RNA) as used in the methods or compositions described herein comprises a portion which is complementary to an mRNA sequence encoding a fidgetin-like 1 protein. In an embodiment, the fidgetin-like 1 protein is a human fidgetin-like 1 protein. In an embodiment, the siRNA is effective to inhibit expression of fidgetin-like 1 protein. In an embodiment, the fidgetin-like 1 protein comprises consecutive amino acid residues having the sequence set forth in SEQ ID NO:29. In an embodiment, the siRNA is effective to inhibit expression of fidgetin protein. In an embodiment, the fidgetin-like 1 protein comprises consecutive amino acid residues having the sequence set forth in SEQ ID NO:28.

In an embodiment, the siRNA that inhibits fidgetin-like 1 comprises a double-stranded portion (duplex). In an embodiment, the siRNA is 20-25 nucleotides in length. In an embodiment the siRNA comprises a 19-21 core RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated. Any of the modifications described herein regarding FL2 are further embodiments of the fidgetin-like 1 inhibitor.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding fidgetin-like 1 protein. In an embodiment, the RNA transcript of a gene encoding fidgetin-like 1 protein is an mRNA. In an embodiment, the fidgetin-like 1 protein is a human fidgetin-like 1 protein. In an embodiment, a siRNA of the invention comprises a double-stranded RNA wherein one strand of the RNA comprises a portion having a sequence the same as a portion of 18-25 consecutive nucleotides of an RNA transcript of a gene encoding fidgetin-like 1 protein. In an embodiment, the fidgetin-like 1 protein is a human fidgetin-like 1 protein. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention inhibiting fidgetin-like 1 is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention inhibiting fidgetin-like 1 comprises at least one 2′-sugar modification. In another embodiment, an siRNA of the invention comprises at least one nucleic acid base modification. In another embodiment, an siRNA of the invention comprises at least one phosphate backbone modification. As used herein, “at least one” means one or more.

In one embodiment, RNAi inhibition of fidgetin-like 1 protein is effected by a short hairpin RNA (“shRNA”). The shRNA is introduced into the appropriate cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene/mRNA, in the present case the mRNA encodes fidgetin-like 1 protein. In an embodiment the fidgetin-like 1 protein is a human fidgetin-like 1 protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In an embodiment, the fidgetin-like 1 is encoded by a nucleic acid sequence comprising the following:

| (SEQ ID NO: 28) | |
|---|---|
| 1 | gtcagtcccc gcgcttttcg gaggctgcca gcgtcccaca ccagccgcag gtaggatgcg |
| 61 | gtgacgggag gcggggcagg ggcgctgctg acccctgtgg agcatcaggg ttggggcgta |
| 121 | ggggagaggc gggcgtgtgc ctctggaatg ctgggggtcg cggggcggtg acttcgcggg |
| 181 | aggccgtggg aggcgggagc caggcagtgg ctgtgccctt ttgcttctgt cttagcggga |
| 241 | gggtggctga cccgctagcc cagaaccaga ctcatccagg attaggaagc tggggcccgg |
| 301 | cgtggacggc aggctcgggg ccaggtctgc gggcggcttc ctttggcaga gcctgtccgg |
| 361 | gggcggggcc gcttaccgca ggcatttact gggcccagcg gggctctggg cgcccacagc |
| 421 | gggggtcagt cagttcccct gtgacccagg caagctctta atctctgtga tgagaaagaa |
| 481 | gggctcatac ctttcagggg tcatcgtgag gaatcagaag aagcagtatt ttatagagta |
| 541 | ttttctaagt gctgagtcct ctgtctagtg ctttgcagaa ttaaaatacc ttgtcagctc |
| 601 | agtgatgttg ctgcgcttct tttataggtg aaaaccggca gaaagacatt aagagatttt |
| 661 | cctgcagtca ctgctggcag atgatagagc caggatttga aagcaggcag cctggctcca |
| 721 | gaccctgtgc tcttaactcc cgttttgcat caagaacaga atcctatgaa aggcttgcac |
| 781 | agtgcttggt actgagtagg cactccgtca gtattttagt agctattatt ccaaaggaca |
| 841 | ctgtattcta ggtactaaga caatgtgaat ggaatggaaa acaattcctc cctgcagaag |
| 901 | ccaactgtct aggaagacag ataatgacat tattaaaata atcagaacat tggcaaaata |
| 961 | aagaagattg aaatgcagtt catatgacag tgctaattaa tcatactttt tagatctagg |
| 1021 | aaaggccata aaggttaaat tgacttggaa atggtaaaca agacaatggt tgtgtcactg |
| 1081 | atcggtgatg gccagttttt ggttatcctt atttgccatt taaatgattg aattgaaagg |
| 1141 | aagaagtaag acttggaaga tgaatattga aggaaaggtg aagatggaaa agagaagggg |
| 1201 | gtttattctg agaacagtga ctagaactct gaggtatggt agaaatttca ggataacaga |
| 1261 | tgataaaggg gaatgaagcc atggagagga agagaatgat aatattaatt gtgaaagttt |
| 1321 | gtgtcaagtg ttttctgagt cacctttctt tcagtttctc atttctagta ttgtataaat |
| 1381 | gagtttaaca tcaaaatggt atagtgaaaa aaaggatgaa ctgcttgaca gaagaggtca |
| 1441 | gatctagttt tgactctagc ttatttagct ttacagcttt aaataaatta cagtgtcttt |
| 1501 | aaagctctgg atcttaagtt ttctaattgt tttttaaaca aagatttgtg aacacctact |
| 1561 | tacaatgtga caaatgtctt tgtaaggcat tatgggagat aaaagctgca ttaaacctgg |
| 1621 | ctgttgtact tacaagtaat ttggaaggca aaatttcaaa tacttgaacc taaagtaggg |
| 1681 | tttcttggtt ttcatttaat tgaaatactg ttaggaaaca aaaatttttc ataaattttt |
| 1741 | cttgtggata tgtaagtaaa ttctatatat ctagcaatat gatacatcta tagttctgtt |
| 1801 | taaaggtaac aatgtgagga agcttatgcg aataacttga cagaagaaat ctaaagtaaa |
| 1861 | agcattttcc tagtaggcta gcttgttctc ttatttcatt tattgtaaca gtttcacttt |
| 1921 | ttaataatta tgtgaaatta ggaaacagat attctagggc attctgttta gggtgatgaa |
| 1981 | cttattcatg aatgttatta aaaccattta aatatgccat agtttaaatt tgtgatccat |
| 2041 | gctgttcaat ctgtgaccta ggatagcagc atcaaggagc attgtgtaca tgcagaagtg |
| 2101 | cacagtacct ggagtgaaac tgcttgtgtt cgatttctga taccattcat aactggctgt |
| 2161 | gtgatctcaa gtaagttctt taagtctctc agcctcggtt tgtacatttg tgtgataagg |
| 2221 | ataatgatag ttttcagatt attttcatag tcattgcttg atttgattct tccaatggtt |
| 2281 | cctctcactt tgcagataag aaaactgaag aaccagctgg gcgcggtagc tcacgcctgt |
| 2341 | aatcccagca ctttgggagg ctgaggcggg tggattacct gaggtcagga gttcaagacc |
| 2401 | agcctgacta acatggcgaa accccatctc tactaaaaat ataaaaatta tccaggggtg |
| 2461 | gtggcaggtg cctgtaatcc cagctacttg ggaagctggg gcaggagaag cacttgaacc |
| 2521 | cgcgaggtgg aggttgcagt gagctgagag cgccactgcc ctccagcagc ctgggtgaca |
| 2581 | gagcgagaca ctgtctcaaa aaaaaataat aataaaataa aaataaacca tcagtgatag |
| 2641 | taaaaagctc acttaagctc attcacctag ttttatagat cagacttaaa cctcttgttt |
| 2701 | attttgtttt gctttatgta tctatagttc cattgttttt aaaatttaaa attaatttta |
| 2761 | aaccttatga atgctttccc tacagataaa attatgtaac tagaacaatt taactcatat |
| 2821 | aaattattat ttaatcaagt ccttaatttt gaagacttca tattgatatg tcttgaatac |
| 2881 | ttgaaatcta tagttaacta gatatttctc ttataacctg tccttataaa tctacattat |
| 2941 | aaataaaaag tccagtgtcc cttccttcag attagcagaa gtttcaaaat tacatttaaa |
| 3001 | gtattttctt tgttttcctt catactgatt ttcattttta ttgtttgtta tagaacctct |
| 3061 | aaaatgcaga cctccagctc tagatctgtg cacctgagtg aatggcagaa gaattacttc |
| 3121 | gcaattacat ctggcatatg taccggaccg aaggcagatg cataccgtgc acagatatta |
| 3181 | cgcattcagt atgcatgggc aaactctgag atttcccagg tctgtgctac caaactgttc |
| 3241 | aaaaaatatg cagagaaata ttctgcaatt attgattctg acaatgttga atctgggttg |
| 3301 | aataattatg cagaaaacat tttaactttg gcaggatctc aacaaacaga tagtgacaag |
| 3361 | tggcagtctg gattgtcaat aaataatgtt ttcaaaatga gtagtgtaca gaagatgatg |
| 3421 | caagctggca aaaaattcaa agactctctg ttggaacctg ctcttgcatc agtggtaatc |
| 3481 | cataaggagg ccactgtctt tgatcttcct aaatttagtg tttgtggtag ttctcaagag |
| 3541 | agtgactcat tacctaactc agctcatgat cgagaccgga cccaagactt cccggagagc |
| 3601 | aatcgtttga aactccttca gaatgcccag ccacctatgg tgactaacac tgctaggact |
| 3661 | tgtcctacat tctcagcacc tgtaggtgag tcagctactg caaaattcca tgtcacacca |
| 3721 | ttgtttggaa atgtcaaaaa ggaaaatcac agctctgcaa aagaaaacat aggacttaat |
| 3781 | gtgttcttat ctaaccagtc ttgttttcct gctgcctgtg aaaatccaca gaggaagtct |
| 3841 | ttttatggtt ctggcaccat tgatgcactt tccaatccaa tactgaataa ggcttgtagt |
| 3901 | aaaacagaag ataatggccc aaaggaggat agcagcctgc ctacatttaa aactgcaaaa |
| 3961 | gaacaattat gggtagatca gcaaaaaaag taccaccaac ctcagcgtgc atcagggtct |
| 4021 | tcatatggtg gtgtaaaaaa gtctctagga gctagtagat cccgagggat acttggaaag |
| 4081 | tttgttcctc ctatacccaa gcaagatggg ggagagcaga atggaggaat gcaatgtaag |
| 4141 | ccttatgggg caggacctac agaaccagca catccagttg atgagcgtct gaagaacttg |
| 4201 | gagccaaaga tgattgaact tattatgaat gagattatgg atcatggacc tccagtaaat |
| 4261 | tgggaagata ttgcaggagt agaatttgct aaagccacca taaaggaaat agttgtgtgg |
| 4321 | cccatgttga ggccagacat ctttactggt ttaaggggac cccctaaagg aattttgctc |
| 4381 | tttggtcctc ctgggactgg taaaactcta attggcaagt gcattgctag tcagtctggg |
| 4441 | gcaacattct ttagcatctc tgcttcatcc ttaacttcta aatgggtagg tgagggggag |
| 4501 | aaaatggtcc gtgcattgtt tgctgttgca aggtgtcagc aaccagctgt gatatttatt |
| 4561 | gacgaaattg attccttgtt atctcaacgg ggagatggtg agcatgaatc ttctagaagg |
| 4621 | ataaaaacag aatttttagt tcaattagat ggagcaacaa catcttctga agatcgtatc |
| 4681 | ctagtggtgg gagcaacaaa tcggccacaa gaaattgatg aggctgcccg gagaagattg |
| 4741 | gtgaaaaggc tttatattcc cctcccagaa gcttcagcca ggaaacagat agtaattaat |
| 4801 | ctaatgtcca aagagcagtg ttgcctcagt gaagaagaaa ttgaacagat tgtacagcag |
| 4921 | attcgcagtt tacaaactgc tgacattgct accataacac cggatcaagt tcgacccata |
| 4981 | gcttacattg attttgaaaa tgcttttaga actgtgcgac ctagtgtttc tccaaaagat |
| 5041 | ttagagcttt atgaaaactg gaacaaaact tttggttgtg gaaagtaagt gggatacttg |
| 5101 | gaatcaaggc atctctgtag tacagtcttc tttatttttt agcatagaaa gttggggatg |
| 5161 | tgttaattgt atttttaaga atatattcta agttctgtac ttcaaataat agcacagatt |
| 5221 | ttacatctga ttgacatagt gtatgttaat gtaagttttg ctttccagtg attacctgat |
| 5281 | acgtaagcct atttgaacaa agtgagaatg aacttttgtt tctaagaagt ctttatcttg |
| 5341 | aagctatata acatgaaaag tgagctcaaa ttttttttag ttgaagatta catataaagt |
| 5401 | tgtgtctgat taatattcat cttttattga agaaagtgcc ttctgatggc cacataattc |
| 5461 | ttaatgtcag ctagtataat ggtttacatt tggacaaagt attgcttagt gttatttaag |
| 5521 | tagatttaag atctcaaagc taaagtgcca atttttactt tcttcagcca atttgttacc |
| 5581 | tcttttatgg tttttaattt ttatcaggac taacattttc agaaatagca aggtgtgatc |
| 5641 | tagtattaga ttcacagaac tgaaaggtta attaagaatg ttggttattt tcacgggagc |
| 5701 | gtgtgatatt taacattaat attttatttg acactacagc ctgtaatact gctctctttc |
| 5761 | aaaaacaagt tttcagattt ttataacaga cccattttgt tttatgaaac atgttcatta |
| 5821 | cagaaacatg agaaaataga gataagtaaa aagaataaaa gtcactatta tcctaccact |
| 5881 | ttgcgggaca cactattaat atttaagcat agattcttcc agatgttttg ttttatacag |
| 5941 | cagtaggatt gtgtaatgta ttctctcttg tagcctattc ttcaaagcgt atcttcactt |
| 6001 | tccatgttaa cataaatgtc cttttatgat tgtctaggat tccatggatg gatggaatat |
| 6061 | aattgagtca tctggtagag cgcatatagg acaggacgct aggttgttta tcttttgttt |
| 6121 | attgtaaagg aactatgtgg gatgcatttg gtgatctttt taaaaaatgt ataaatttct |
| 6181 | tgagtaattc ctggattaaa gaatatgcac tttttaaagc ttttggcttg cattctaaat |
| 6241 | tgtcctcttg aaaaagtagt tcctgtgtaa aactccatct gcactgaatc agagctatgg |
| 6301 | cagcattggg cagtaggcag ttttggtatt tgttggcatt ctttttgcat ttattcaaaa |
| 6361 | aactgaaggt agtgaacgta gagcaatgaa caacacaaat atccttggtc tcgtggaggt |
| 6421 | tatatttaag ttctaatgtt ttctcttgtg aacagtgcaa tttgaaatga caaattgaaa |
| 6481 | cacttttagt tgttgaattt agacagtttg tgtatatagt ttaaattttt attcttggtt |
| 6541 | tgccttctgt ttttatgtta aaagattgtt gcaattttta gtttttccct ttgtaatatg |
| 6601 | tgattgtctt tcattagaat tcattttttg ttttgttttg gttattttga aacagtctcg |
| 6661 | ctgtgttgcc taggctggag tgcagtggtg tgatctcagc tcactgcaac ctctgcctcc |
| 6721 | tgggttccag tgattcttgt gcctcagtct cccttgtagc tgagaccaca ggtttgcacc |
| 6781 | accacaccca gctaattttt gtatttttaa tcgagatggg gtttcaccat gttgcccagg |
| 6841 | ctgctctcga actcctggcc tcaaacgatc cacccacctc agcctcctga agtgttggga |
| 6901 | ttacaggcat gagccaccac gcctggcctt ttttattaga atttggacgt taaacttctt |
| 6961 | gtcttcagtt cttagttata tcttttaaca tcctaaaaca ttccacaata ttcattactg |
| 7021 | tctttatccc tttctatttc tatataaaca gcactataaa cttggtttct ttggtattga |
| 7081 | tgtcaaggaa ttctctagtt tttctttctt ggtgtttaat tgcagatcac tcccaccttc |
| 7141 | ttgggtctag ctatttagat ttatcagcat ttgccctcta gctgttaatg ttttatttag |
| 7201 | ctctgaaatt tggtcttaat atttattatc agtttttttc agttacagct tctgccagcc |
| 7261 | ctgactttaa gctcccttga tcaccagcca cagggccact tctttctatg gagtttagaa |
| 7321 | attgtgacaa ggagagaaag tctagttggt ttagctcatc ttggattaag cagagcccat |
| 7381 | gtctctcttg ttagctcaag aaatggtctt tcctgaatta aggtctactc tgttaaataa |
| 7441 | gaatctaaac ctgaaaggaa tagtcaggat attttaaagc tggaaagaaa atttgagctg |
| 7501 | gggagtcaac tgttgtttct acgttatacc acagagaagc tttctttgtc tcaacacttt |
| 7561 | gtgattttga gtgtttctga agcagttgga taggaagctt actgaaactt taagattgct |
| 7621 | tactctcgcc acatgcgatt gtcttcatcg aatataataa tcttggatta aattatctca |
| 7681 | agtgtggcac tgccctgtct ttagctattg tttcagaaac atgaatgcta gcctgatatt |
| 7741 | taatgtcttc aaaaattaca ttcccttagt agaaatacac attttactaa acatgtcact |
| 7801 | tggttatggt attaatgctt aacaattttg atatgaatat ggtaattcta atttatgatt |
| 7861 | tcaggccttc ttaaatcttt attatacatc taataataat atatgatcct tttgatatgt |
| 7921 | attcattctg tggtaaattt ctaattttct gaaattttaa ctcattttct caattctaaa |
| 7981 | atatctctca ggtgtgcctt tcatattgac aagctggtct taatggaatc atagtctaaa |
| 8041 | gatcatgggc tgtggagcca aatagacctg ttgaattctg gctctgtcat ttactggtga |
| 8101 | ctgttggtta cttaattatt caatctcaat tctaaagtga gaatagttta tacctgtctt |
| 8161 | gttggagagt gaggattaaa tagtaaaata cagatttaaa aggcctggtt catgttttat |
| 8221 | gattgtgcct cttctttgaa tctgccttga ttttgctctt gttgctgtga aggatctttt |
| 8281 | cagctatttt gtgtggttgt cctagctcat tctgcttagt gtttgtggta tttgcatcat |
| 8341 | attattagag atcatatgcc atctatcttt agttttgtgg aaaaattcta attccaaaga |
| 8401 | aatatctctt agttatgttt ctccttttct acactttaga acttttttaa aggtctgtcc |
| 8461 | cagattatta tttttctcta ctcatgctta tatgttattt attggtctcc atttttactc |
| 8521 | tcccagtgtt tttttaaggg acactttaca gcccaccttt agggatctga ttaaaagaaa |
| 8581 | ggattctatc aaggtccatg gcaaaaggac aggttggata aagataacat ctttttactt |
| 8641 | ccttcttata ttcctagttc ttgaccttag ttggggagct ggtggtgagg gagcacattt |
| 8701 | taaacagttt aagcaggtga ggccatcttg aaggtttgcc ccagcagcct gggtgaatgg |
| 8761 | agggagtctt gggagaatag ataagatgca gtcaccactt ttaactcttg cccttaaaaa |
| 8821 | atacttttac aacacggctt ataatggaaa gagcattaaa atggacatta gaaaactgcc |
| 8881 | agtgattcta accttagaga gtttgtacag actccgtaag atttgttttt ttagtgataa |
| 8941 | agggaaattg tactggtcat tccttaataa cacatccaga tttctgttat cacttaccaa |
| 9001 | aaatttatga atttgagaat tgcttacaca gtatcaggtg ctgggtactg ggaggtacaa |
| 9061 | aacaaagtat agtcaggtaa gggctaactt tattactaat ttggctcatt ataaaaacaa |
| 9121 | cgggaaaaca tgaagagagg gctttattaa ggagttaagt cagcataatg gaccacttaa |
| 9181 | tatataataa ccacttaaga gaaaaagaaa aattaggaag tccacataag gtggcgtttt |
| 9241 | ataaaggcaa catgctgttg ttaagggcac ttgggccatt ggtgttttgt gacagctggg |
| 9301 | ctcaggcatc tcaacaagca gcggtgtgcc tgctgaccaa cagggcctgc cacggccagt |
| 9361 | gaaacacagc tattagagct ctctcaatcc atttctgctc agaaatgtag agatggcagg |
| 9421 | ggaaacgtag ctaagtccca ggttcctcgg atatggcgag ggcaagcact cccatattat |
| 9481 | aacaacctgt gcagtcagtg agtccagagc agaggccttg tctcctcctt tgctcacctt |
| 9541 | cactcactgc cctggactgt caggccccag ttctcatccc catgtgtgat gaagccatgc |
| 9601 | agtcccagag agggagccat cagcgcttgg tagtctcatg ctttagtgtc agagttgcac |
| 9661 | cttacctttg tatccaagaa atgcatgcag ctcaactttc ccttaaaagg cttctaggaa |
| 9721 | aaaactaccc cagcattttt tttcccccag aagaatatat tagcttaata ataaaaggta |
| 9781 | tagtaacaaa ctttccattt aaaatagtgg cctaagccac aaatttacct ttcctgcccg |
| 9841 | tatgtgcaaa attttcattg aaatgaaaaa atacacacta agaatcaatc cagtgtgatg |
| 9901 | ttggaaaaga gagaaggtta ccattttcaa atatgacagg tgtttctgga ggtcacaagg |
| 9961 | caaatggtac tgggttgttg gaatccctga gctaaataat ggctgtgaaa atgccagatg |
| 10021 | agattggtta tctcagcagt gcctgggctg tacccctagc ttaggcagca aaataggctg |
| 10081 | tgggccagtg gtctggtatc tgaaaggatt gccaagacaa gccttgcgga gctcatgccc |
| 10141 | aactgttctt gtggaaccac tgggaagtgg agttccctaa atcaggggga gtcatttaca |
| 10201 | gtcgcctggc ttttgtgggc cctgcagcct gagaagtgag tcaggatggc aggtacttgg |
| 10261 | ttgccacgtt agacagaagc acaagtattt gatgaagcag tggaacttct tgatgtaact |
| 10321 | cactcctgtc tcggtaactt tttacctact tccacatttt tttaagggtc gacccccact |
| 10381 | tcctacagtt tctctatatc cttgccactg acacatgcag gcttacgcca agacatagag |
| 10441 | gaggtcttgc tcacgccaca gttaacacat ctctatccat ggctgggcaa tatgaaagac |
| 10501 | aattgctaag gaagctttct cctgtcagca tactgatcac ttttaaagta taacttaaca |
| 10561 | ttacttcccc ttgtaaaaat cctccaatag attcccatct aaatgaaaat aaaatccaaa |
| 10621 | ttccttccta gagccaaata tcatttctct tcactctcct ttcttacttt ccttcatccc |
| 10681 | actggatctt aaggtgcaac aaacatgttt ctcatatagg aagtgtggta ggcagaattc |
| 10741 | tgaagatgtt cctccaagag acccatcccc tgcttattca gtcaaatcta ggtactgctg |
| 10801 | ggaagggact ttgcagaggt aatgcggtta cagacctgca gatagggagg ttatcttgaa |
| 10861 | ttatttgtgt agggccaatc taatttcaca agtccttaaa agcagagaat gttctctggc |
| 10921 | tggttagaga agaggagggc taaagaggca gaagaaagat gtggcagaga ggatgtccaa |
| 10981 | gattccgagc atgagaagga ttttctctga agttgcagct ctgtgaatca ggagcccatg |
| 11041 | cacgaaggct ggagagaagt ctctaggagc tgacaggagc ctccagacaa tagccaacaa |
| 11101 | aaaccagagg tgtcagtcct agaactgcat agaagggaat tctgccagca acctaaataa |
| 11161 | gccaggaagc ctcacccaga ggttccagat gagaatccag gccggctgac accttgattt |
| 11221 | cagtcttatg ggacccagag cagagaaact ggtagagact cctatacttc tgatgtacag |
| 11281 | aactgtgggg tcataactgt gtgtggttct aagctgctaa gtttatggta gtttgttaca |
| 11341 | gcagcaatgt ggggtaggga acattacact gttctttgag tgtggaaagc tcttccttac |
| 11401 | agctgctgct tcaagttagt aaaatataat ttctccagca agacctcctt tgaccactgg |
| 11461 | ggagatagcc tccactaact tccaacacgt gcatgccatc actatcatat tatcctacat |
| 11521 | tttttattgg taactgttac taactgaaaa tacttatgct tactgtcttt gtctctaatc |
| 11581 | ctatttcaca cccattagaa agttctttac agctagggac tttatgtgca cttcacaacc |
| 11641 | atgcaagatt catgagactt ctgaaaaacc caacatcatg agaggcaccc agagaagaag |
| 11701 | cagagtgatt cccatcaaag aaaaccattg attcaggaga ctgagtagag ctttagaaag |
| 11761 | tctcgatccc agaagacatg gtagccacca aattaaaata ggcagctatt gtattagtat |
| 11821 | attgttacat aacaatatca ccacaaactt agtgacttta aacaacacac attaatgacc |
| 11881 | ttgtagtttc tttcaggctt agctgggtcc tctgcttcag ggacccacag ggctgcaatg |
| 11941 | aaggtgttgg tcaggactgt ggtctcatct gaggcttgtt ggggaaggat ccacttctca |
| 12001 | gctcacatgg tggttgggca acattcagtt tcttacagat ggctggacag agggcctcag |
| 12061 | ttcttgccat ctgttggctg gaggttgccc tcagttcctt gccatgtagg cattccagtg |
| 12121 | tggctgcttg cttcctcaaa gccggcaggg gagagagcct ttccagtagc acagatgtta |
| 12181 | ctatcttctg taatgtaatc acacaggcaa cactttgtca tctttgctgt attctgtagg |
| 12241 | gtgagagcaa gacacaggtc cttcccactc ttagggaagg gtattaaaaa tgacaactac |
| 12301 | caggaggtag ggatcatgga gcctgcttca gaatctatca cagctgtgaa ataaaggccc |
| 12361 | aggaaacaag aattctaatg gttaaaataa aaatcttagt ggaagaactg aatttttaaa |
| 12421 | atggaaaggg ctgaaacaag atagttaaaa ggaagaataa gctgtggaat tattttacaa |
| 12481 | tcttcagcag acagagctag agtgaggagt taagtagtat gacagattcc aggtaactaa |
| 12541 | tatccatata atggggaatc tatagaaagt aaaaaggaga gaggtagatg ataattgcat |
| 12601 | tattgagagc agataattct tctgagtgaa atacatagtt catatcaaaa gcatctactg |
| 12661 | agtgtgtggt gttatatata tatgtaggtt tttcatccag ggttctttgg ctcataactc |
| 12721 | ccacagccct tacacagtct tgttataatg ttggatgtgt taggcctcag gtaggcctct |
| 12781 | ggtgttctgc cctccttcca ctctaatatt ctcccacctt tctgattgtg ggtcttaaga |
| 12841 | ctcaaaattc caagaggaca gggttcagag agcttcccaa tagctgaaca tgtggaggtt |
| 12901 | ccgggagagt ggtgtgccca ggtagggcat ggaagcttca agcctcttcc cccatacctt |
| 12961 | gccctagcgt ttcttcgtct gtattttttc cagtatagag caataaactg gtgaactaag |
| 13021 | tgtt. |

The sequence of human fidgetin-like 1 protein is:

(SEQ ID NO: 29)
MQTSSSRSVHLSEWQKNYFAITSGICTGPKADAYRAQILRIQYA
WANSEISQVCATKLFKKYAEKYSAIIDSDNVESGLNNYAENILT
LAGSQQTDSDKWQSGLSINNVFKMSSVQKMMQAGKKFKDSLLEP
ALASVVIHKEATVFDLPKFSVCGSSQESDSLPNSAHDRDRTQDF
PESNRLKLLQNAQPPMVTNTARTCPTFSAPVGESATAKFHVTPL
FGNVKKENHSSAKENIGLNVFLSNQSCFPAACENPQRKSFYGSG
TIDALSNPILNKACSKTEDNGPKEDSSLPTFKTAKEQLWVDQQK
KYHQPQRASGSSYGGVKKSLGASRSRGILGKFVPPIPKQDGGEQ
NGGMQCKPYGAGPTEPAHPVDERLKNLEPKMIELIMNEIMDHGP
PVNWEDIAGVEFAKATIKEIVVWPMLRPDIFTGLRGPPKGILLF
GPPGTGKTLIGKCIASQSGATFFSISASSLTSKWVGEGEKMVRA
LFAVARCQQPAVIFIDEIDSLLSQRGDGEHESSRRIKTEFLVQL
DGATTSSEDRILVVGATNRPQEIDEAARRRLVKRLYIPLPEASA
RKQIVINLMSKEQCCLSEEEIEQIVQQSDAFSGADMTQLCREAS
LGPIRSLQTADIATITPDQVRPIAYIDFENAFRTVRPSVSPKDL
ELYENWNKTFGCGK.

Non-limiting examples of siRNA targeting fidgetin-like 1 include:

(SEQ ID NO: 30)
AGUGAUUACCUGAUACGUA,
(SEQ ID NO: 31)
GUGCACAGAUAUUACGCAU,
(SEQ ID NO: 32)
GAAUAAGGCUUGUAGUAAA
OR
(SEQ ID NO: 33)
GGAUCAAGUUCGACCCAUA.

Any of the foregoing sequence may be provided as a double stranded nucleic acid with a complementary sequence. Such siRNA molecules may be provided in any of the compositions described herein and used for any of the purposes described herein for FL2.

The nucleic acid sequences described herein may be prepared by any method known in the art, and purified by HPLC or any other method to provide inhibitors suitable for use for the in vitro, ex vivo or in vivo purposes described herein. In some embodiments, the purity of the inhibitor is equal to or greater than 85%. In some embodiment the purity is equal to or greater than 90%. In some embodiments the purity is equal to or greater than 95%. In some embodiment the purity is equal to or greater than 98%. In some embodiments the purity is equal to or greater than 99%. In some embodiments wherein the inhibitor is or comprises a duplex, the purity of the duplex is equal to or greater than 85%. In some embodiments wherein the inhibitor is or comprises a duplex, the purity of the duplex is equal to or greater than 90%. In some embodiments wherein the inhibitor is or comprises a duplex, the purity of the duplex is equal to or greater than 95%. In some embodiments wherein the inhibitor is or comprises a duplex, the purity of the duplex is equal to or greater than 98%. In some embodiments wherein the inhibitor is or comprises a duplex, the purity of the duplex is equal to or greater than 99%. In some embodiments the inhibitor is prepared under current Good Manufacturing Practices. In some embodiments the inhibitor is prepared for human use. In some embodiments the inhibitor is prepare for in vitro or ex vivo use for subsequent administration to humans. In some embodiments the inhibitor is prepared for human administration.

### Delivery of FL2 Inhibitor Agents

As described herein, the one or more agents that inhibit FL2 in one embodiment is delivered in vivo, in order to increase the homing to or penetration of T-cells to lymph nodes, tumor sites or into tumors. In one embodiment the agent is delivered to or near a lymph node of the patient.

In one embodiment a wafer comprising the FL2 inhibitor agent such as siRNA is implanted at a site. In one embodiment, a composition of the FL2-siRNA-wafer is 2.5% collagen, 7.5% chondroitin sulfate, 82.5% polyvinylpyrrolidone (PVP) and 7.5% PEG400. siRNA will be incorporated into the wafer, and the siRNA will be measured and optimized, for example for size (by scanning electron microscopy, light scattering and atomic force microscopy), pH, charge, rate of delivery, amount of polyplex delivered, minimum fill and leakage, in order to obtain a suitable formulation to achieve good delivery and efficacy of siRNA knockdown. Formulations to be tested will be applied to the site of surgery at the time of CAR-T injection. The FL2-siRNA-wafers (or controls containing nonsense-siRNA) are a gel-like matrix that will be cut to the appropriate size and applied with forceps to the site of surgery.

In another embodiment, the siRNA may be delivered by an implantable delivery device, such as described in Zhang et al., 2019, Sci Adv 5:eaaw5296, incorporated herein by reference.

In one embodiment, albumin-binding phospholipid polymers may be used to traffic inhibitors from blood into lymph. Other examples are described in Liu et al. 2014, Nature 543:113-117. Methods for delivering agents to lymph nodes may be found in Ma et al., Science 2019 Jul. 12; 365(6449):162-168, incorporated herein by reference.

In another embodiment, the agent is used ex vivo or in vitro to induce subsequently infused T-cells to be better able to home to or penetrate into tumor sites and tumors. In one embodiment, the agent is delivered to T cells. In one embodiment, the agent is a RNA interference agent such as a siRNA or shRNA. In one embodiment, the siRNA agent is delivered to T cells by a method such as but not limited to electroporation/nucleofection, Accel siRNA, viral vectors, peptides, proteins, nanoparticles and aptamers. Delivery of siRNA to T cells may be achieved by methods described in Freeley et al., Biochemical Journal 2013 Oct. 15; 455(2):133-147, incorporated herein by reference. Some examples and literature references are provided for guidance but are not intended to be limiting.

Electroporation/nucleofection. Electroporation is a frequently-used physical method for nucleic acid transfer. In this method, the cells and nucleic acid suspended in a special buffer are subjected to high voltage pulses of electricity which generates a potential difference across the membrane and induces temporary pores in the cell membrane. Arabsolghar et al., 2012, Iran J Med Sci 37(3):187-193 described electroporation in more detail. Nucleofection is an electroporation-based transfection method which enables transfer of nucleic acids such as DNA and RNA into cells by applying a specific voltage and reagents. One non-limiting example is described in Iversen et al., 2005, Genet Vaccines Ther 3:2.

The CAR-T manufacturing process is generally amenable to electroporation/nucleofection based knock down for patient derived T-cell (see, for example, https://www.stemcell.com/media/files/wallchart/WA27041-Production_of_Chimeric_Antigen_Receptor_T_cells.pdf) Thus, FL2 knockdown can be conducted during manufacturing. For sustaining knockdown after manufacturing, either a CRISPR based knockout of FL2 in T cells can be performed. In another embodiment, siFL2 wafer can be introduced into lymph nodes.

Accel siRNA. Accel siRNA is a technique provided by Dharmacon to achieve efficient RNA silencing. It is described at https://dharmacon.horizondiscovery.com/rnai/sirna/accell/#overview.

Viral vector. A viral vector may be used to deliver RNA interference nucleic acids such as siRNA. See Tomar et al., 2003, Oncogene 22(36):5712-5715 and Kurreck et al., 2017, J RNAi Gene Silencing 13:545-547.

Peptides and proteins. Cell penetrating peptides and proteins may be used to deliver nucleic acids such as siRNA and shRNA into cells. See, for example, Cummings et al., 2019, Transl Res, in press; Ni et al., 2019, Life (Basel) 9(9); Crombez et al., Biochem Soc Transactions 35(1):44-46; and Pottash et al., 2019, J Biol Engineer 13:19.

Aptamers. Aptamers may also be used to deliver nucleic acids such as siRNA and shRNA into cells. See Krupse et al., 2017, Biomedicines, 5(3):45 and Chu et al., 2006, Nucl Acids Res 34(10):e73.

Nanoparticles. In one embodiment, a nanoparticle is used to deliver the agent to T cells. Preparation of siRNA delivering nanoparticles is known in the art, such as described in Kim et al., 2019, Adv Mater (49)e1903637, epub; Ickenstein et al., 2019, Expert Opin Drug Deliv 16(11): 1205-1226 (Sep. 17 epub). Various methods may be employed to prepare nanoparticles for effective deliver of their payload siRNA or shRNA into T cells.

In one embodiment, nanoparticles comprising tetramethyl orthosilicate (TMOS) are used, wherein the TMOS nanoparticles comprise FL2 siRNA. For example, five hundred μl of tetramethyl orthosilicate (TMOS) can be hydrolyzed in the presence of 100 μl of 1 mM HCl by sonication on ice for about 15 min, until a single phase forms. The hydrolyzed TMOS (100 μl) can then be added to 900 μl of 20 μM of siRNA (mouse FL2 (Sigma-Aldrich, SASI_Mm02_00354635) or a negative control) solution containing 10 mM phosphate, pH 7.4. A gel is formed within 10 minutes. The gel may be frozen at −80° C. for 15 minutes and lyophilized.

The fidgetin, fidgetin-like 1 or FL2 inhibitor agent may be used in a composition with additives. Examples of suitable additives are sodium alginate, as a gelatinizing agent for preparing a suitable base, or cellulose derivatives, such as guar or xanthan gum, inorganic gelatinizing agents, such as aluminum hydroxide or bentonites (termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. Hydrophobic organogels are also suitable. Hydrophobic additives, such as petroleum jelly, wax, oleyl alcohol, propylene glycol monostearate and/or propylene glycol monopalmitostearate, in particular isopropyl myristate can be included. In an embodiment the inhibitor is in a composition comprising one or more dyes, for example yellow and/or red iron oxide and/or titanium dioxide for the purpose of matching as regards color. Compositions may be in any suitable form including gels, lotions, balms, pastes, sprays, powders, bandages, wound dressing, emulsions, creams and ointments of the mixed-phase or amphiphilic emulsion systems (oil/water-water/oil mixed phase), liposomes and transfersomes or plasters/band aid-type coverings. Emulsifiers which can be employed in compositions comprising the inhibitor of fidgetin-like 2 include anionic, cationic or neutral surfactants, for example alkali metal soaps, metal soaps, amine soaps, sulphonated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

Compositions comprising the agent that inhibits fidgetin, fidgetin-like 1 or fidgetin-like 2 can also comprise vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats (for example based on caprylic acid, capric acid, lauric acid or stearic acid, such as Softisan®), or triglyceride mixtures, such as Miglyol®, can be used as lipids, in the form of fatty and/or oleaginous and/or waxy components for preparing the ointments, creams or emulsions of the compositions comprising the inhibitor of fidgetin-like 2 used in the methods described herein.

Osmotically active acids and alkaline solutions, for example hydrochloric acid, citric acid, sodium hydroxide solution, potassium hydroxide solution, sodium hydrogen carbonate, may also be ingredients of the compositions and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions are additional forms of compositions comprising the agent that inhibits of fidgetin, fidgetin-like 1 or fidgetin-like 2. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

In an embodiment, the compositions comprise further active ingredients, for example one or more antibiotics, antiseptics, vitamins, anesthetics, antihistamines, anti-inflammatory agents, moisturizers, penetration-enhancing agents and/or anti-irritants.

In an embodiment of the methods and compositions described herein the subject is a mammal. In an embodiment the subject is human.

In one embodiment, nanoparticles comprising the RNA interference agent are used for delivery for either the in vitro, ex vivo or in vivo applications of the methods described herein.

### Tumors

In one embodiment, the methods described herein are used to increase or enhance the homing of T cells to, or increase the penetration of T cells into tumor sites or tumors including solid tumors. In one embodiment, the methods described herein are used to a liquid tumor. In one embodiment, the methods described herein are used to liquid tumors hiding in solid tissues (see, for example, “Leukaemia cells hide in fat tissue,” Nature (2016) 535:11). Any tumor or cancer is a potential site for the methods of the invention. Any organ or solid organ of the body that is a potential site for a tumor or cancer is a site for the methods described herein. Non-limiting examples of tumors include tumors that comprise esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

### Diagnostic Methods

The methods of enhancing T cell homing and penetration into tumors described herein may in some embodiments be combined with diagnostic methods prior to, during, or following T cell therapy for the purposes, by non-limiting example, including determining need for enhanced T cell homing or penetration activity; and determining effectiveness of enhancing T cell homing or penetration activity. In one embodiment, the diagnostic test can indicate whether the dosing level or frequency of administration of FL2 inhibition therapy is effective or should be increased, or can be decreased.

In one embodiment, the extent of tumor homing and/or penetration of T cells of any source described herein is first determined, wherein those T cells needing enhancement of homing or penetration activity are subsequently exposed to an agent as described herein. In one embodiment, T cell migration activity towards tumor cells, tumor antigen, or cells expressing tumor antigen is evaluated in vitro, using methods similar to those described in the examples herein, where a sample of T cells obtained during the ex vivo or in vitro preparation for infusion are evaluated for homing or tumor (spheroid) penetration activity and such information used to direct, for example, the decision to and extent of FL2 inhibition of the T cell population before infusion into the patient. Other non-limiting examples of methods for assessing homing or penetration activity may be found in Sherman et al., 2018, Front Immunol April 2018, 9:article 857; https://www.corning.com/media/worldwide/cls/documents/applications/CLS-AN-447%20DL.pdf; and https://www.corning.com/media/worldwide/cls/documents/CLS-PST-055_CAR-T_Cell_Screening_Tumor_Spheroids.pdf.

For example, during tumor biopsy to obtain T cells and identify the patient's tumor antigen for preparation of CAR-T cells, tumor cells if available, or other cells transfected to express the tumor antigen, are used to set up a 3D gel migration assay, in which T cells undergoing CAR modification and expansion are evaluated in vitro for attraction to and penetration into cells expressing the patient's tumor antigen. The extent of attraction and/or penetration is used to determine whether the CAR-T cells should be treated with the FL2 inhibitor before infusion into the patient.

The following examples are presented in order to more fully illustrate the preferred embodiments of the invention. It should in no way be construed, however, as limiting the broad scope of the invention.

### EXAMPLES

### Experimental Approach

The Jurkat cell line was used as a model for T-cells. The prostate cancer cell lines DU145 and LnCAP were used as models for cancer cells. The T-cells and cancer cells were first co-cultured and observed in a 3D gel migration assay. Subsequently, prostate cancer spheroids were used to model a chemoattractant that has a hypoxic tumor microenvironment for the T-cells in a 3D gel-based co-culture.

The levels of FL2 were then downregulated in T-cells by an siRNA-mediated approach. The Jurkat cells were nucleofected with FL2 siRNA or control siRNA using the standard protocol provided by Lonza (https://bioscience.lonza.com/lonza_bs/US/en/download/product/asset/21051) . The concentration of siRNA was 100 nM. Alternative methods may be carried out with other steps as suggested by Freely M, Long A, Advances in siRNA delivery to T-cells: potential clinical applications for inflammatory disease, cancer and infection, Biochem J (2013) 455(2):133-147. The number of T-cells migrating towards the spheroids was quantified.

The FL2 siRNA and control siRNA were nucleofected into Jurkat cells (cells were grown to a density of 3×10\hat{ }5 cells/ml before Nucleofection). In the procedure, the required number of cells (1×10\hat{ }6) are centrifuged at 90×g for 10 minutes at room temperature. The supernatant was removed completely and the cell pellet resuspended carefully in room temperature in 4DNucleofector™ Solution. The siRNA and GFP reporter plasmids were added and subjected to the nucleofection process. The resulting suspension was mixed with pre-warmed DMEM medium. The cells were incubated in humidified 37° C./5% CO2 incubator until analysis. Gene expression or down regulation, respectively, is often detectable after only 4-8 hours. Protein level knockdown was confirmed after 48-72 hours using western blotting.

### Example 1. Assessing Migration of T Cells Toward Cancer Cells

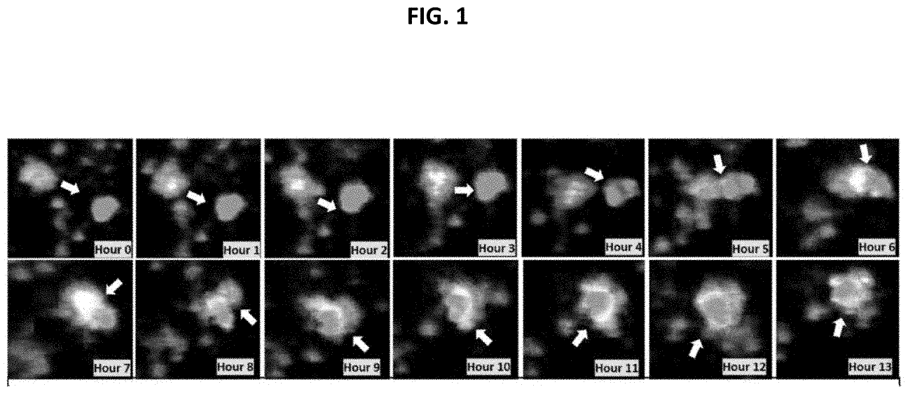

As shown in FIG. 1, time-lapse images demonstrate migration of T-cells (red) towards a cancer cell (green) in a 3D co-culture of Jurkat cells (T-cell line) with DU145 cells (prostate cancer cell line). The T-cells (red) show a directional movement (arrows) towards small clusters of cancer cells (green) (hour 0 to hour 5) and with time show phagocytosis of the cancer cells (hour 6 to hour 13). Both the cell lines were pre-stained with membrane specific lipophilic Vybrant dyes and then co-cultured in a 3D Matrigel plug.

### Example 2. Assessing Migration of T Cells Toward Cancer Spheroids

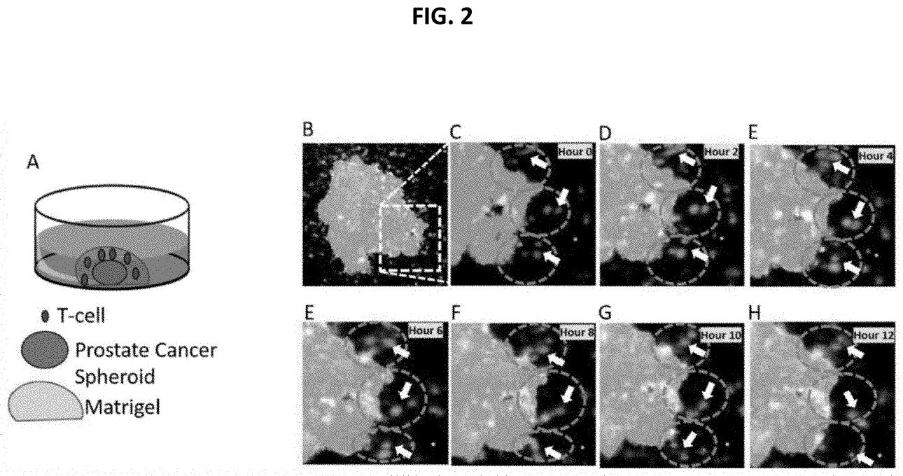

Subsequently, using prostate cancer spheroids, limited penetration of T-cells was observed (FIG. 2A). The Jurkat cells (T-cell line) were co-cultured in a Matrigel plug along with a prostate cancer spheroid derived from DU145 cell line. FIG. 2B. Low magnification image of pre-stained Spheroid (green) and the surrounding pre-stained T-cells (red). FIG. 2C to FIG. 2H: Time lapse series tracking the motility and penetration of T-cells (red). Note the migration of T-cells directed towards the edge of spheroid (arrows) (FIG. 2C-FIG. 2F) and then migration away from the spheroid possibly owing to immunosuppressive hypoxic tumor microenvironment.

### Example 3. FL2 Knockdown Enhances T Cell Migration Towards Cancer Spheroids

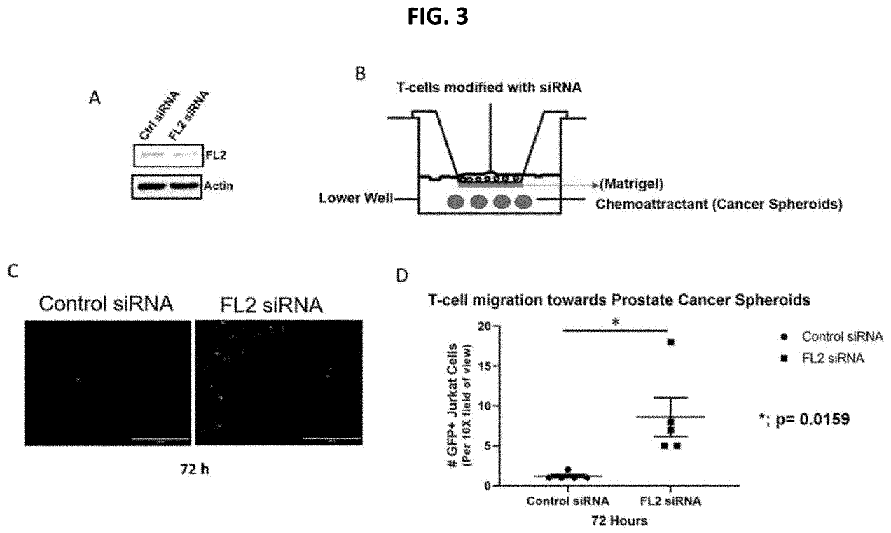

To evaluate the effect of FL2 knockdown, Jurkat cells were exposed to siRNA to FL2. GFP was included to identify the knocked-down cells. FIG. 3A shows by Western blot the knockdown of FL2 in Jurkat cells (T-cell line). A trans-well assay was established (FIG. 3B) for quantifying T-cell migration towards cancer spheroids. The T-cells modified with siRNA+GFP are placed in the upper chamber and the spheroids are in the lower chamber. The trans-well semipermeable membrane is coated with Matrigel to trap the T-cells migrating towards the spheroids. FIG. 3C shows by imaging the GFP-positive cells on the transwell membrane 72 hours after co-culture with cancer spheroids (Scale bar=400 um), and in FIG. 3D, quantification of GFP-positive T-cells from images show significantly (*; p=0.0159) higher number of T-cells treated with FL2 siRNA migrating towards the cancer spheroids.

### Example 4. FL2 Knockdown Enhances T Cell Migration Towards Cancer Spheroids

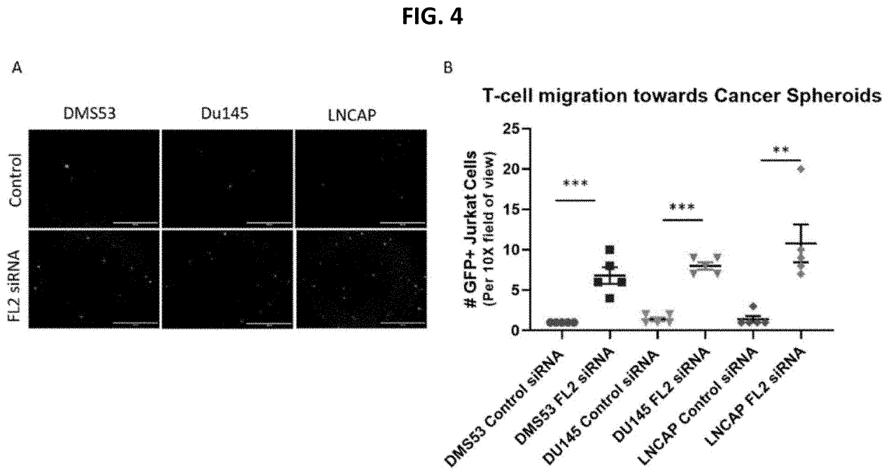

To demonstrate the effect of FL2 knockdown of T-cells enhances migration to different cancer spheroids, FIG. 4A shows imaging the GFP-positive cells on the transwell membrane 72 hours after co-culture with cancer spheroids from prostate cancer cell lines (DU145 and LnCAP) and lung cancer cell line (DMS53) (Scale bar=400 um). In FIG. 4B, quantification of GFP-positive T-cells from images show significantly (***; p<0.001; **; p<0.01) higher number of T-cells treated with FL2 siRNA migrating towards the cancer spheroids.

### Example 5. Studies in Preclinical Models

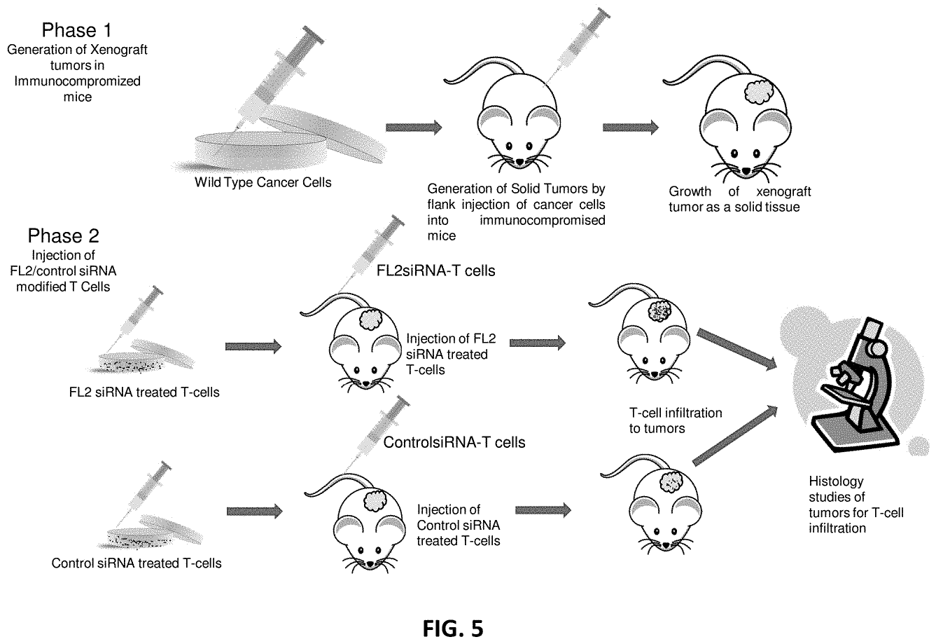

The experimental procedure to identify the role of FL2 downregulation in directing T-cells to infiltrate tumors in higher numbers in vivo as compared to control is shown in FIG. 5. The experimental design will utilize primary CD8+cytotoxic T cells (e.g., ATCC® PCS-800-017™) and T cell lines SUP-T1 [VB] (ATCC® CRL-1942™), MOLT-4 (ATCC® CRL-1582™) and TALL-104 (ATCC® CRL-11386™). Briefly, in study Phase 1, the cancer cells e.g., Du145, DM553) are injected in the flank region of immunocompromised mice through intra-peritoneal injection and allowed to generate solid tumors. In a parallel experiment (Phase 2), T-cells from a human T cell line (for example, as described above) are genetically engineered for FL2 knockdown/knockout, for example by nucleofection as described herein) and then grown for 48-72 hours in vitro. These T-cells will be injected in through tail vein in the tumor bearing mice of Phase 1. The tumors are resected and probed for markers of T-cells in the tumor tissues. It is anticipated that FL2 knockdown of FL2 in infused T cells will improve anti-cancer therapy in the model.

### Example 6. Treatment of Cancer Using FL2 Knockdown of T Cells

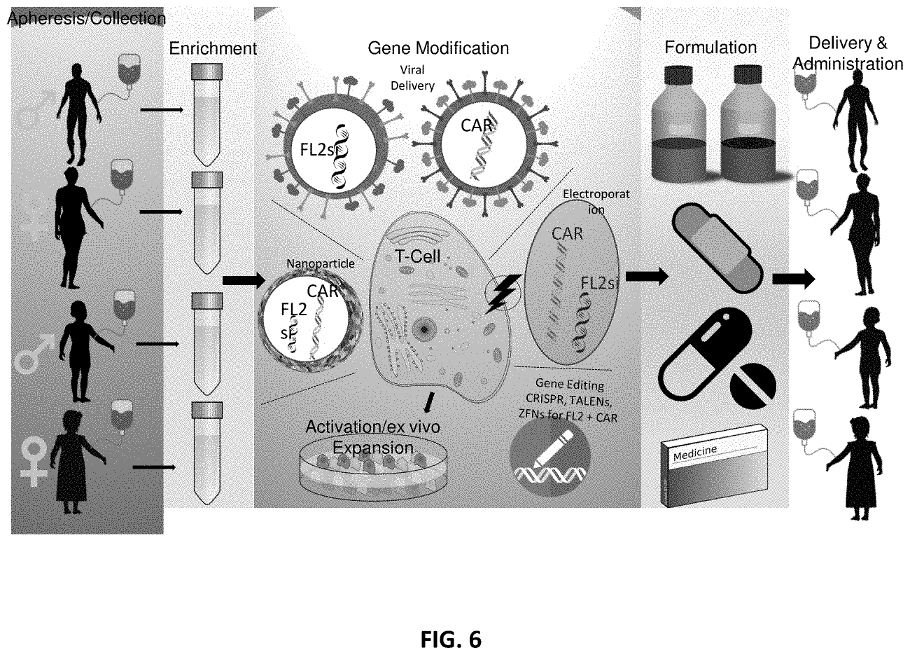

In human patients, CAR-T cells (shown in FIG. 6) are generated with FL2-modification using multiple strategies which include co-delivery of CAR and FL2 siRNA genetic elements through nanoparticles, viruses, electroporation and gene-editing methods (such as but not limited to CRISPR, TALENs, ZFNs) as described herein. Subsequent to this, the cell therapies are formulated and delivered to the respective patients. Improved killing of tumor cells by T cells that have FL2 inhibition is demonstrated.

In other studies, FL2 knockdown of T cells isolated and expanded from a patient's tumor is also shown to increase T cell killing of tumor cells in solid tumors and blood cancers.

In the experiments described here, the T cells engineered for CAR to target solid tumors/blood cancers will have increased motility due to FL2 knockdown. Increased T cell motility will result in a larger numbers of T-cell interacting with cancer cells in solid tumors or blood cancer cells penetrated in solid tissues such as lymph nodes. The consequence of higher T cell numbers will lead to faster reduction of tumor mass (i.e., tumor shrinkage).

In practice, autologous T cells will be isolated from patient blood and engineered for CAR and FL2 ex vivo. The modified T cells will be injected back into the patient. In some studies modified T cells will be exposed to checkpoint inhibitors and/or other factors ex vivo. The patient may also be treated with checkpoint inhibition therapy. The patients will then be monitored for T-cell infiltration into the tumor (using biopsy) and size of tumor using MRI.

### Example 7. FL2 Knockdown Increases T Cell Attachment to Tumor Spheroids

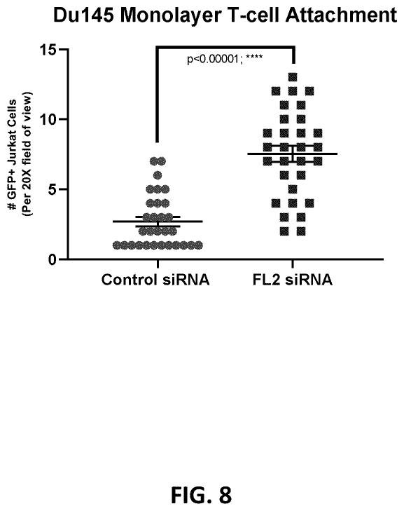

FIG. 8 shows the effect of FL2 knockdown on T cell attachment to a Du145 monolayer. The experiment is designed to quantify T cell attachment to the solid tumor surface. The surface of a solid tumor is demonstrated as a monolayer of Du145 (a prostate cancer cell line). The T cell line, Jurkat, is used as a model for suspension cell culture where FL2 knockdown is carried out. The objective is to study the role of FL2 in enhancing the transition of T-cells from suspension to adherent mode in a co-culture condition. The T cells treated with control or FL2 siRNA were incubated with a monolayer of Du145 cells for 24 hours. The T cells have a GFP marker for identification through imaging. After co-culture, the unadhered T-cells were removed and the monolayer of Du145 was washed with PBS. The attached T-cells were imaged and counted per 20× field of view under a microscope. (FIG. 8) The comparison of control siRNA vs FL2 siRNA treated T-cells shows a significant increase (p<0.00001) in the number of T-cells attached to a monolayer of Du145 cells for those with FL2 knockdown.

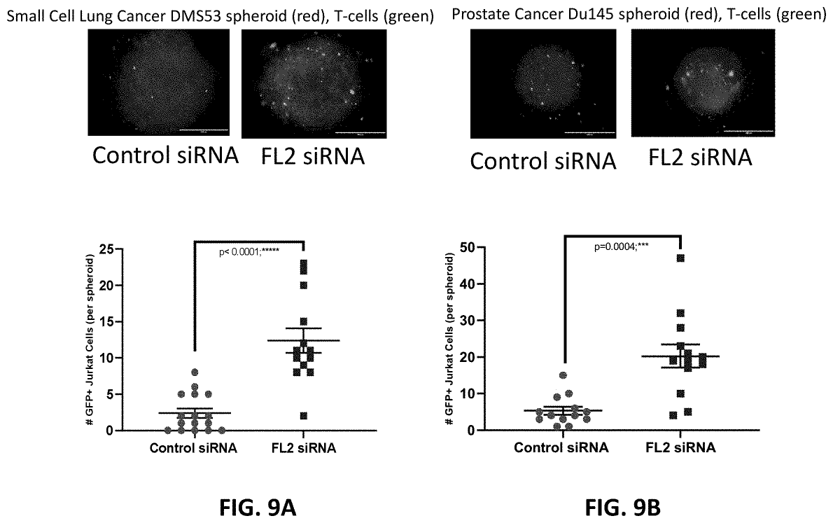

The results described in FIG. 8 were verified by a 3D co-culture system. FIG. 9 depicts a study of the attachment of T cells to a tumor spheroid. 3D tumor spheroids were grown in low-attachment 96-well plates using a prostate cancer cell line (Du145) and a small-cell lung cancer cell line (DMS53). After 48-72 h, the T cells treated with control or FL2 siRNA were incubated with individual spheroids of Du145 or DMS53 cells for 24 hours. After co-culture, the unadhered T-cells were removed and the spheroids of Du145 or DMS53 were washed with PBS and imaged under a microscope.

FIG. 9 A-B show the effect of FL2 knockdown on attachment of T cells to tumor spheroids. The comparison of number of T-cells (green) attached to spheroids (red) is significantly higher when FL2siRNA is used for T-cells on DMS53 (FIG. 9A) or Du145 (FIG. 9B). The results indicate that both kind of solid tumors, T-cell penetration can be increased significantly by knockdown of FL2.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for enhancing the tumoricidal activity of T cells comprising the step of reducing fidgetin-like 2 expression or activity in the T cells.

2. The method of claim 1 wherein the tumoricidal activity is against a solid tumor, a liquid tumor, a bone marrow tumor or a blood cancer.

3. The method of claim 1 wherein the tumoricidal activity is against a carcinoma, a sarcoma, a lymphoma, a leukemia, a myeloma or a mixed type tumor.

4. The method of claim 1 wherein enhancing the tumoricidal activity comprises enhancing the migration of T cells toward a tumor site or tumor cells, enhancing the penetration or infiltration of T cells into a tumor, enhancing the penetration or infiltration of T cells into a bodily site comprising tumor cells, or any combination thereof.

5. The method of claim 1 wherein the T cells are endogenous T cells or adoptive T cell therapy.

6. The method of claim 4 wherein the T cells are autologous T cells, CAR-T cells, tumor infiltrating lymphocytes, engineered T cell receptor lymphocytes, macrophages, microglia or natural killer cells, or are derived from lymphoid progenitor cells or pluripotent stem cells.

7. The method of claim 1 wherein reducing fidgetin-like 2 expression or activity in the T cells is carried out in vivo, ex vivo or in vitro.

8. The method of claim 7 wherein the in vivo reducing fidgetin-like 2 expression or activity comprises administering an inhibitor of fidgetin-like 2 to a subject parenterally, into a tissue, into an organ, lymph node, intratumorally or adjacent to a tumor.

9. The method of claim 7 wherein the ex vivo reducing fidgetin-like 2 expression or activity comprises exposing T cells ex vivo to an inhibitor of fidgetin-like 2.

10. The method of claim 9 wherein the T cells are subsequently infused into a subject or a site within the subject.

11. The method of claim 7 wherein the in vitro reducing fidgetin-like 2 expression or activity comprises exposing T cells in vitro to an inhibitor of fidgetin-like 2.

12. The method of claim 1 wherein the T cells are autologous, allogeneic, lymphoid progenitors or pluripotent stem cells.

13. The method of claim 1 wherein the T cells are obtained from a cell line or from a donor.

14. The method of any one of claims 8-13 wherein the inhibitor of fidgetin-like 2 is a RNA interference agent.

15. The method of claim 14 wherein the RNA interference agent is shRNA or siRNA

16. The method of claim 15 wherein the siRNA has a sequence selected from
(SEQ ID NO: 1)
UUACACAGUAUUAAAGCGAUU;
(SEQ ID NO: 2)
UCGCUUUAAUACUGUGUAAUU;
(SEQ ID NO: 3
CAUCUGAAACCUAGGGUCUUU;
(SEQ ID NO: 4)
AGACCCUAGGUUUCAGAUGUU;
(SEQ ID NO: 5)
GUGACUUAUGCUAGGAGGAUU;
(SEQ ID NO: 6)
UCCUCCUAGCAUAAGUCACUU;
(SEQ ID NO: 7)
GGUCAGAAGCAGAAUGUAUUU;
(SEQ ID NO: 8)
AUACAUUCUGCUUCUGACCUU;
(SEQ ID NO: 9)
CGCCGGCCCACAAGUUGGAdTdT;
(SEQ ID NO: 10)
UCCAACUUGUGGGCCGGCGdTdT;
(SEQ ID NO: 11)
CAGCUCGAGCCCUUUGACAdTdT;
(SEQ ID NO: 12)
UGUCAAAGGGCUCGAGCUGdTdT;
(SEQ ID NO: 13)
CCUCCAACCUCCUCAAGAGdTdT;
(SEQ ID NO: 14)
CUCUUGAGGAGGUUGGAGGdTdT;
(SEQ ID NO: 15)
CGUUGCUGCUCAUCAGCGAdTdT;
(SEQ ID NO: 16)
UCGCUGAUGAGCAGCAACGdTdT;
(SEQ ID NO: 17)
fUfUmA fCmAfC AGU AUU AAA GCG ATT;
(SEQ ID NO: 18)
(Phos) U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAmCf
UmGfUmGfUmAmATT-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmA
fCmUGmUmGfUmAmATT;
(SEQ ID NO: 42)
5′mUmUmAmCmAmCmAmGmUmAmUmUmAm
AmAmGmCmGmAmUmU-3′;
(SEQ ID NO: 43)
(Phos) 5′-mUmCmGmCmUmUmUmAmAmUm
AmCmUmGmUmGmUmAmAmUmU-3′;
(SEQ ID NO: 44)
5′mUmUmAmCmAmCmAmGmUmAmUmUmAmAm
AmGdCdGdATT-3′;
(SEQ ID NO: 45)
5′mUmUmAmCmAmCmAmGmUmAmUmUmAmAm
+0AmGdCmGmATT-3′;
(SEQ ID NO: 46)
5′UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate.

17. The method of claim 15 wherein the siRNA has at least one modification selected from a 3′ overhang, a 5′ overhang, a 5′ phosphorylation, a 2′ sugar modification, a nucleic acid base modification, a phosphate backbone modification, and any combination of any of the foregoing.

18. The method of claim 1 wherein the fidgetin-like 2 is human fidgetin-like 2.

19. The method of claim 15 wherein the siRNA is encapsulated in a nanoparticle.

20. The method of claim 15 wherein the siRNA is delivered by nanoparticles, electroporation/nucleofection, Accel siRNA, a viral vector, peptide, protein or aptamer.

21. The method of any one of claim 6, 8 or 11 wherein an immune checkpoint inhibitor is administered to the subject, or the T cells are exposed to an immune checkpoint inhibitor, ex vivo or in vitro.

22. The method of claim 2 wherein the tumor or cancer is colorectal cancer, pancreatic cancer, liver cancer, intrahepatic bile ductal cancer, esophageal cancer, bladder cancer, non-Hodgkin's lymphoma or kidney cancer.

23. A method for treating cancer in a subject in need thereof comprising administering to the subject a population of T cells wherein the expression or activity of fidgetin-like 2 therein has been reduced.

24. The method of claim 23 the cancer comprises a solid tumor, a liquid tumor, a bone marrow tumor or a blood cancer.

25. The method of claim 23 wherein the cancer is a carcinoma, a sarcoma, a lymphoma, a leukemia, a myeloma or a mixed type tumor.

26. The method of claim 23 wherein the T cells are adoptive T cell therapy.

27. The method of claim 26 wherein the population of T cells are autologous T cells, CAR-T cells, tumor infiltrating lymphocytes, engineered T cell receptor lymphocytes, macrophages, microglia or natural killer cells, or are derived from lymphoid progenitor cells or pluripotent stem cells.

28. The method of claim 23 wherein reducing fidgetin-like 2 expression or activity in the T cells is carried out ex vivo or in vitro.

29. The method of claim 28 wherein the ex vivo reducing fidgetin-like 2 expression or activity comprises exposing T cells ex vivo to an inhibitor of fidgetin-like 2 prior to administration to the subject.

30. The method of claim 28 wherein the in vitro reducing fidgetin-like 2 expression or activity comprises exposing T cells in vitro to an inhibitor of fidgetin-like 2 prior to administration to the subject.

31. The method of claim 28 wherein the T cells are obtained from a cell line or a donor.

32. The method of claim 29 wherein the inhibitor of fidgetin-like 2 is a RNA interference agent.

33. The method of claim 32 wherein the RNA interference agent is shRNA or siRNA.

34. The method of claim 33 wherein the siRNA has a sequence selected from
(SEQ ID NO: 1)
UUACACAGUAUUAAAGCGAUU;
(SEQ ID NO: 2)
UCGCUUUAAUACUGUGUAAUU;
(SEQ ID NO: 3
CAUCUGAAACCUAGGGUCUUU;
(SEQ ID NO: 4)
AGACCCUAGGUUUCAGAUGUU;
(SEQ ID NO: 5)
GUGACUUAUGCUAGGAGGAUU;
(SEQ ID NO: 6)
UCCUCCUAGCAUAAGUCACUU;
(SEQ ID NO: 7)
GGUCAGAAGCAGAAUGUAUUU;
(SEQ ID NO: 8)
AUACAUUCUGCUUCUGACCUU;
(SEQ ID NO: 9)
CGCCGGCCCACAAGUUGGAdTdT;
(SEQ ID NO: 10)
UCCAACUUGUGGGCCGGCGdTdT;
(SEQ ID NO: 11)
CAGCUCGAGCCCUUUGACAdTdT;
(SEQ ID NO: 12)
UGUCAAAGGGCUCGAGCUGdTdT;
(SEQ ID NO: 13)
CCUCCAACCUCCUCAAGAGdTdT;
(SEQ ID NO: 14)
CUCUUGAGGAGGUUGGAGGdTdT;
(SEQ ID NO: 15)
CGUUGCUGCUCAUCAGCGAdTdT;
(SEQ ID NO: 16)
UCGCUGAUGAGCAGCAACGdTdT;
(SEQ ID NO: 17)
fUfUmA fCmAfC AGU AUU AAA GCG ATT;
(SEQ ID NO: 18)
(Phos) U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAm
CfUmGfUmGfUmAmATT-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmAfCmUGmUmGfUmAmATT;
(SEQ ID NO: 42)
5′mUmUmAmCmAmCmAmGmUmAmUmUmAm
AmAmGmCmGmAmUmU-3′;
(SEQ ID NO: 43)
(Phos) 5′-mUmCmGmCmUmUmUmAmAmUmAmCm
UmGmUmGmUmAmAmUmU-3′;
(SEQ ID NO: 44)
5′mUmUmAmCmAmCmAmGmUmAmUmUmAmAmAmGdCdGdATT-3′;
(SEQ ID NO: 45)
5′mUmUmAmCmAmCmAmGmUmAmUmUmAmAmAmGdCmGmATT-3′;
(SEQ ID NO: 46)
5′UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate.

35. The method of claim 32 wherein the siRNA has at least one modification selected from a 3′ overhang, a 5′ overhang, a 5′ phosphorylation, a 2′ sugar modification, a nucleic acid base modification, a phosphate backbone modification, [others], and any combination of any of the foregoing.

36. The method of claim 23 wherein the fidgetin-like 2 is human fidgetin-like 2.

37. The method of claim 28 wherein the siRNA is in a wafer or encapsulated in a nanoparticle.

38. The method of claim 28 wherein the siRNA is delivered by nanoparticle, electroporation/nucleofection, Accel siRNA, a viral vector, peptide, protein or aptamer.

39. The method claim 23 wherein an immune checkpoint inhibitor is administered to the subject.

40. The method of claim 23 wherein the cancer is colorectal cancer, pancreatic cancer, liver cancer, intrahepatic bile ductal cancer, esophageal cancer, bladder cancer, non-Hodgkin's lymphoma or kidney cancer.

41. A method for treating cancer in a subject in need thereof comprising administering to or near a lymph node or tumor or tumor site within the subject an inhibitor of the expression or activity of fidgetin-like 2.

42. The method of claim 41 the cancer comprises a solid tumor, a liquid tumor, a bone marrow tumor or a blood cancer.

43. The method of claim 41 wherein the inhibitor of fidgetin-like 2 is a RNA interference agent.

44. The method of claim 43 wherein the RNA interference agent is shRNA or siRNA.

45. The method of claim 44 wherein the siRNA has a sequence selected from
(SEQ ID NO: 1)
UUACACAGUAUUAAAGCGAUU;
(SEQ ID NO: 2)
UCGCUUUAAUACUGUGUAAUU;
(SEQ ID NO: 3
CAUCUGAAACCUAGGGUCUUU;
(SEQ ID NO: 4)
AGACCCUAGGUUUCAGAUGUU;
(SEQ ID NO: 5)
GUGACUUAUGCUAGGAGGAUU;
(SEQ ID NO: 6)
UCCUCCUAGCAUAAGUCACUU;
(SEQ ID NO: 7)
GGUCAGAAGCAGAAUGUAUUU;
(SEQ ID NO: 8)
AUACAUUCUGCUUCUGACCUU;
(SEQ ID NO: 9)
CGCCGGCCCACAAGUUGGAdTdT;
(SEQ ID NO: 10)
UCCAACUUGUGGGCCGGCGdTdT;
(SEQ ID NO: 11)
CAGCUCGAGCCCUUUGACAdTdT;
(SEQ ID NO: 12)
UGUCAAAGGGCUCGAGCUGdTdT;
(SEQ ID NO: 13)
CCUCCAACCUCCUCAAGAGdTdT;
(SEQ ID NO: 14)
CUCUUGAGGAGGUUGGAGGdTdT;
(SEQ ID NO: 15)
CGUUGCUGCUCAUCAGCGAdTdT;
(SEQ ID NO: 16)
UCGCUGAUGAGCAGCAACGdTdT;
(SEQ ID NO: 17)
fUfUmA fCmAfC AGU AUU AAA GCG ATT;
(SEQ ID NO: 18)
(Phos) U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAmCfUm
GfUmGfUmAmATT-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmAfCmU
GmUmGfUmAmATT;
(SEQ ID NO: 42)
5′mUmUmAmCmAmCmAmGmUmAmUmUmAmA
mAmGmCmGmAmUmU-3′;
(SEQ ID NO: 43)
(Phos) 5′-mUmCmGmCmUmUmUmAmAmUmAmCm
UmGmUmGmUmAmAmUmU-3′;
(SEQ ID NO: 44)
5′mUmUmAmCmAmCmAmGmUmAmUmUmAm
AmAmGdCdGdATT-3′;
(SEQ ID NO: 45)
5′mUmUmAmCmAmCmAmGmUmAmUmUmAmAmAmGdCmGmATT-3′;
(SEQ ID NO: 46)
5′UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate.

46. The method of claim 44 wherein the siRNA has at least one modification selected from a 3′ overhang, a 5′ overhang, a 5′ phosphorylation, a 2′ sugar modification, a nucleic acid base modification, a phosphate backbone modification, [others], and any combination of any of the foregoing.

47. The method of claim 41 wherein the fidgetin-like 2 is human fidgetin-like 2.

48. The method of claim 44 wherein the siRNA is in a wafer or encapsulated in a nanoparticle.

49. The method of claim 41 wherein an immune checkpoint inhibitor is administered to the subject.

50. The method of claim 41 wherein the cancer is colorectal cancer, pancreatic cancer, liver cancer, intrahepatic bile ductal cancer, esophageal cancer, bladder cancer, non-Hodgkin's lymphoma or kidney cancer.

51. The method of claim 41 wherein the inhibitor is infused paratumorally or intratumorally.

52. The method of claim 41 wherein the subject concurrently receives or previously received T cell therapy.

53. A method for treating cancer in a subject in need thereof comprising the steps of (1) determining whether the activity of T cells to migrate to or penetrate into a solid tumor is reduced, and if such activity is reduced, (2) exposing T cells in vitro or ex vivo to an inhibitor that reduces the expression or activity of fidgetin-like 2, and administering the T cells to the subject, or administering to or near a lymph node, tumor or tumor site within the subject, an inhibitor that reduces the expression or activity of fidgetin-like 2, or any combination thereof.

54. The method of claim 53 wherein the T cells are endogenous T cells or the T cells administered for adoptive T cell therapy.

55. The method of claim 53 wherein the determining whether the activity of endogenous T cells or T cells for administration for adoptive T cell therapy to migrate or penetrate into a solid tumor is carried out by a T cell migration or penetration assay.

56. The method of claim 53 wherein the cancer is a solid tumor, a liquid tumor, a bone marrow tumor or a blood cancer.

57. The method of claim 53 wherein the cancer is a carcinoma, a sarcoma, a lymphoma, a leukemia, a myeloma or a mixed type tumor.

58. The method of claim 53 wherein exposing enhances the migration of T cells toward a tumor site or tumor cells, enhances the penetration or infiltration of T cells into a tumor, enhances the penetration or infiltration of T cells into a bodily site comprising tumor cells, or any combination thereof.

59. The method of claim 53 wherein the T cells are administered for adoptive T cell therapy.

60. The method of claim 59 wherein the T cells are autologous T cells, CAR-T cells, tumor infiltrating lymphocytes, engineered T cell receptor lymphocytes, macrophages, microglia or natural killer cells, or are derived from lymphoid progenitor cells or pluripotent stem cells.

61. The method of claim 53 wherein reducing fidgetin-like 2 expression or activity in the T cells is carried out in vivo, ex vivo or in vitro.

62. The method of claim 61 wherein the in vivo reducing fidgetin-like 2 expression or activity comprises administering an inhibitor of fidgetin-like 2 to a subject parenterally, into a tissue, into an organ, lymph node, intratumorally or adjacent to a tumor.

63. The method of claim 61 wherein the ex vivo reducing fidgetin-like 2 expression or activity comprises exposing T cells ex vivo to an inhibitor of fidgetin-like 2.

64. The method of claim 63 wherein the T cells are subsequently infused into a subject or a site within the subject.

65. The method of claim 61 wherein the in vitro reducing fidgetin-like 2 expression or activity comprises exposing T cells in vitro to an inhibitor of fidgetin-like 2.

66. The method of claim 65 wherein the T cells are subsequently infused into a subject or a site within the subject.

67. The method of claim 53 wherein the T cells are autologous, allogeneic, lymphoid progenitors or pluripotent stem cells.

68. The method of claim 53 wherein the T cells are obtained from a cell line or from a donor.

69. The method of any one of claims 62-68 wherein the inhibitor of fidgetin-like 2 is a RNA interference agent.

70. The method of claim 69 wherein the RNA interference agent is shRNA or siRNA.

71. The method of claim 70 wherein the siRNA has a sequence selected from among SEQ ID NOs:1-18 or 34-57.

72. The method of claim 70 wherein the siRNA has at least one modification selected from a 3′ overhang, a 5′ overhang, a 5′ phosphorylation, a 2′ sugar modification, a nucleic acid base modification, a phosphate backbone modification, and any combination of any of the foregoing.

73. The method of claim 53 wherein the fidgetin-like 2 is human fidgetin-like 2.

74. The method of claim 70 wherein the siRNA is encapsulated in a nanoparticle.

75. The method of claim 70 wherein the siRNA is delivered by nanoparticles, electroporation/nucleofection, Accel siRNA, a viral vector, peptide, protein or aptamer.

76. The method of any one of claim 60, 62 or 65 wherein an immune checkpoint inhibitor is administered to the subject.

77. The method of claim 53 wherein the tumor or cancer is colorectal cancer, pancreatic cancer, liver cancer, intrahepatic bile ductal cancer, esophageal cancer, bladder cancer, non-Hodgkin's lymphoma or kidney cancer.

78. The method of claim 53 wherein an immune checkpoint inhibitor is administered to the subject or exposed to T cells ex vivo or in vitro before administration to the subject.

79. A method for enhancing the tumoricidal activity of T cells comprising the step of reducing fidgetin expression or activity in the T cells.

80. A method for treating cancer in a subject in need thereof comprising administering to a subject a population of T cells wherein the expression or activity of fidgetin therein has been reduced.

81. A method for treating cancer in a subject in need thereof comprising administering to or near a lymph node or tumor or tumor site within the subject an inhibitor of the expression or activity of fidgetin.

82. A method for treating cancer in a subject in need thereof comprising the steps of (1) determining whether the activity of T cells to migrate to or penetrate into a solid tumor is reduced, and if such activity is reduced, (2) exposing T cells in vitro or ex vivo to an inhibitor that reduces the expression or activity of fidgetin, and administering the T cells to the subject, or administering to or near a lymph node, tumor or tumor site within the subject, an inhibitor that reduces the expression or activity of fidgetin, or any combination thereof.

83. A method for enhancing the tumoricidal activity of T cells comprising the step of reducing fidgetin-like 1 expression or activity in the T cells.

84. A method for treating cancer in a subject in need thereof comprising administering to a subject a population of T cells wherein the expression or activity of fidgetin-like 1 therein has been reduced.

85. A method for treating cancer in a subject in need thereof comprising administering to or near a lymph node or tumor or tumor site within the subject an inhibitor of the expression or activity of fidgetin-like 1.

86. A method for treating cancer in a subject in need thereof comprising the steps of (1) determining whether the activity of T cells to migrate to or penetrate into a solid tumor is reduced, and if such activity is reduced, (2) exposing T cells in vitro or ex vivo to an inhibitor that reduces the expression or activity of fidgetin-like 1, and administering the T cells to the subject, or administering to or near a lymph node, tumor or tumor site within the subject, an inhibitor that reduces the expression or activity of fidgetin-like 1, or any combination thereof.
